(19) United States
(12) Patent Application Publication
(10) Pub. No.: US 2016/0199853 A1
(43) Pub. Date: Jul. 14, 2016

(54) APPARATUS AND METHOD FOR MANIPULATION OF DISCRETE POLARIZABLE OBJECTS AND PHASES

(71) Applicant: UNIVERSITY OF WASHINGTON THOUGH ITS CENTER FOR COMMERCIALIZATION, Seattle, WA (US)

(72) Inventors: Melissa M. Harwood, Seattle, WA (US); Robbyn K. PERDUE, Seattle, WA (US); Eleanor S. JOHNSON, Seattle, WA (US)

(21) Appl. No.: 14/914,917

(22) PCT Filed: Aug. 28, 2014
(86) PCT No.: PCT/US14/53242

§ 371 Date: Feb. 26, 2016

Related U.S. Application Data
(60) Provisional application No. 61/872,431, filed on Aug. 30, 2013.

(51) Int. Cl. B03C 5/00; B03C 5/02; G01N 27/447; G01N 27/27; C12N 13/00; B01L 3/00

(52) U.S. Cl. CPC B03C 5/005; C12N 13/00; B01L 3/50273; B01L 3/502715; B01L 3/502761; G01N 27/44756; G01N 27/27; B03C 5/026; B01L 2400/0424; B01L 2300/161; B01L 2300/165; B01L 2300/0861; B01L 2200/0647; B01L 2200/06

(57) ABSTRACT

Methods, systems, and devices for manipulating objects are provided. In certain aspects, the methods, systems, and devices can be used for dielectrophoretic manipulation of objects using bipolar electrodes. Some aspects of the methods, systems, and devices of the present disclosure can be used for encapsulation and amplification of samples.

### CROSS-REFERENCE

This application claims the benefit of U.S. Provisional Application No. 61/872,431, filed Aug. 30, 2013, the disclosure of which is herein incorporated by reference in its entirety.

### STATEMENT AS TO FEDERALLY SPONSORED RESEARCH

This invention was made with the support of the United States government under National Cancer Institute grant number T32CA138312 and Department of Defense grant number BC100510(W81XWH-11-1-0814).

### BACKGROUND

Over the past decade, the scientific community has become increasingly attuned to heterogeneity in seemingly homogeneous cell populations. Even among clonal cells, stochastic events lead to variations in gene expression and diverse responses to endogenous and exogenous stimuli. Cellular heterogeneity has documented impact in many fields of research such as the rare induction of somatic cells into pluripotent stem cells, division of labor in neighboring neurons, and varied drug response. Heterogeneity within cancer cell populations is of special interest for cancer treatment strategies because a minority of drug resistant cells can seed cancer recurrence after “clinical cure.” None of these processes can be studied effectively using ensemble measurements, and therefore, highly sensitive analytical tools are needed for probing single cells.

There is a need to provide improved methods and apparatuses for performing object manipulation. The present disclosure addresses this need and more.

### SUMMARY

The present disclosure provides methods, systems, and devices for manipulating objects using dielectrophoretic forces.

In various aspects, the present disclosure provides dielectrophoretic systems comprising: a fluidic containment structure comprising an ionically conductive phase; a bipolar electrode having a portion situated within the fluidic containment structure, the portion being in electrical communication with the ionically conductive phase; and a power source in electrical communication with the ionically conductive phase and configured to apply an electric field thereto, the electric field comprising an AC component having a frequency range from about 1 kHz to about 100 MHz and a voltage range from about 1 V to about 1 kV and a DC component having a voltage range from about 10 mV to about 100 V.

In various aspects, the present disclosure provides fluidic devices comprising: a first fluidic channel comprising a first ionically conductive phase; a second fluidic channel comprising a second ionically conductive phase; a bipolar electrode comprising a first portion and a second portion, wherein the first portion is in electrical communication with the first ionically conductive phase and the second portion is in electrical communication with the second ionically conductive phase; and a power source in electrical communication with the first and second ionically conductive phases and configured to apply an electric field comprising an AC component and a DC component to the first and second ionically conductive phases, the electric field comprising an electric field minimum or an electric field maximum near the first and second portions of the bipolar electrode.

In various aspects, the present disclosure provides fluidic devices comprising: a plurality of fluidic containment structures each comprising an ionically conductive phase; a plurality of bipolar electrodes each comprising a first portion and a second portion, wherein the first portion of each of the plurality of bipolar electrodes is in electrical communication with an ionically conductive phase of one of the plurality of fluidic containment structures and the second portion of each of the plurality of electrodes is in electrical communication with an ionically conductive phase of another of the plurality of fluidic containment structures; and a power source configured to apply an electric field comprising an AC component and a DC component to each ionically conductive phase of the plurality of fluidic containment structures, the electric field comprising electric field minima or electric field maxima near the first and second portions of each of the plurality of bipolar electrodes.

In various aspects, the present disclosure provides methods for manipulating an object comprising: providing a fluidic containment structure comprising an ionically conductive phase and a bipolar electrode comprising a portion in electrical communication with the ionically conductive phase; applying an electric field comprising an AC component and DC component to the ionically conductive phase, wherein the electric field comprises an electric field minimum or an electric field maximum near the portion of the bipolar electrode; introducing an object into the ionically conductive phase; and manipulating the position of the object within the ionically conductive phase using the electric field minimum or electric field maximum.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A is a diagram of a single channel BPE device.

FIG. 1B is a detailed picture of negative dielectrophoretic force on an object at the BPE of FIG. 1A.

FIG. 1C is a graph of electric field strength over the BPE of FIG. 1A with and without faradaic reactions.

FIG. 1D is a graph of the derivative of the electric field over the BPE of FIG. 1A, illustrating the negative dielectrophoretic trap.

FIG. 1E is a graph of the electric potential over the BPE of FIG. 1A, illustrating the overpotential at the ends of the BPE.

FIG. 1F illustrates a top down view of the BPE of FIG. 1A in the channel showing attraction of objects.

FIG. 2 illustrates an array of BPEs with two large driving electrodes.

FIG. 3A illustrates a dual-channel BPE configuration.

FIG. 3B is a graph of electric potential over the BPE in the dual-channel configuration of FIG. 3A.

FIG. 4A is a graph of the x-component of electric field strength over one end of a BPE contacting two channels.

FIG. 4B illustrates a U-shaped BPE contacting two channels.

FIG. 4C illustrates a pair of BPEs contacting two channels.

FIG. 5 illustrates a comb-like interdigitate BPE array.

FIG. 6A illustrates a linear positive dielectrophoresis BPE array.

FIG. 6B illustrates electric field lines near a BPE of FIG. 6A.

FIG. 7 illustrates a dual-channel BPE device.

FIG. 8A illustrates dielectrophoresis of polarizable objects in a non-uniform electric field.

FIG. 8B is a graph of the impact of faradaic ion enrichment and faradaic ion depletion on the electric field.

FIG. 9 illustrates a portion of a device for concentration enrichment via dielectrophoresis in a cylindrical channel with a pair of ring-shaped BPEs.

FIG. 10 illustrates a device for trapping an object within a chamber.

FIG. 11 illustrates a device for directing an object into an outlet channel.

FIG. 12 illustrates a serpentine channel BPE array.

FIG. 13A illustrates a multi-channel device including an interdigitate BPE array.

FIG. 13B illustrates a close view of a portion of FIG. 13A.

FIG. 14A illustrates a device including a membrane array of BPEs.

FIG. 14B illustrates the membrane array of FIG. 14A.

FIG. 15A is a top view of a device for trapping objects in a well format.

FIG. 15B is a side view of the device of FIG. 15A.

FIG. 16A is a perspective view of a portion of a device for trapping objects in a well format.

FIG. 16B is a side view of the device of FIG. 16B.

FIG. 17A illustrates a device that can be used for transport of cell lysis products into a constriction or side channel.

FIG. 17B illustrates capture of a cell at the BPE tip in the device of FIG. 17A.

FIG. 17C illustrates cell swelling and membrane disruption following cell capture in FIG. 17B.

FIG. 17D illustrates transport of cell contents through a constriction to another channel following lysis in FIG. 17C.

FIGS. 18A through 18G illustrate a device for trapping and lysis of cells in isolated chambers.

FIG. 19 illustrates a device for segmenting a sample solution into droplets.

FIGS. 20A through 20C illustrate combined electrophoretic and negative dielectrophoretic capture at a BPE anode, followed by release after turning off the electric field.

FIGS. 21A through 21C illustrate multiple cell capture with combined electrophoresis and negative dielectrophoresis at a BPE anode.

FIGS. 22A through 22C illustrate combined electrophoretic and negative dielectrophoretic cell capture at a BPE anode followed by osmotically induced cell swelling when switching voltage to create ion depletion. Cell is stained blue with Trypan blue after cell membrane disruption.

FIG. 23 is a series of optical micrographs which show negative dielectrophoretic repulsion of a B-cell from the BPE tip under AC-only electric field in Tris dielectrophoresis buffer. Each image slice (numbered sequentially 1-5) is separated by 2.5 s. E_{RMS,avg}=5 kV/m (t=0 s) to 17.7 kV/m (t=5 s). ω=1.8 kHz.

FIGS. 24A through 24C are multiple series of optical micrographs showing increasing negative dielectrophoretic attraction of a B-cell toward the BPE anode in Tris dielectrophoresis buffer. E_{DC,avg}=0.75 kV/m; E_{RMS,avg}=5 kV/m (FIG. 24A), 13.3 kV/m (FIG. 24B), 17.7 kV/m (FIG. 24C). Image slices are 1 s apart. ω=1.8 kHz.

FIG. 24D illustrates negative dielectrophoretic attraction of a B-cell toward the BPE cathode in phosphate dielectrophoresis buffer (4 s/slice). E_{DC,avg}=0.75 kV/m, E_{RMS,avg} increased from 5.7 kV/m to 28.3 kV/m from t=0 s (slice 1) to t=8 s (slice 3). ω=1.8 kHz.

FIG. 24E illustrates release of the trapped cells (2 s/slice) from FIG. 24D upon subsequent decrease of E_{RMS,avg} to 5.7 kV/m (from slice 1 to slice 2). ω=1.8 kHz.

FIGS. 25A through 25D are multiple series of optical micrographs which demonstrate negative dielectrophoretic repulsion of individual B-cells from a faradaic ion depletion zone at the BPE cathode in Tris dielectrophoresis buffer (0.5 s/slice). E_{DC,avg}=1.25 kV/m; E_{RMS,avg}=0.57 kV/m (FIG. 25A), 6.13 kV/m (FIG. 25B), 7.95 kV/m (FIG. 25C), 10.25 kV/m (FIG. 25D). ω=1.8 kHz.

FIG. 26 is a series of optical micrographs showing negative dielectrophoretic repulsion of B-cells from a faradaic ion depletion zone formed at the BPE anode in phosphate DEP buffer (1 s/slice). Arrows indicate one of the repelled cells. E_{RMS,avg}=8.0 kV/m and E_{DC,avg}=2.5 kV/m. ω=1.8 kHz.

FIGS. 27A and 27B are sequential optical micrographs showing negative dielectrophoretic and electrophoretic repulsion of B-cells from a faradaic ion depletion zone at the BPE cathode in Tris dielectrophoresis buffer (E_{Dc,avg}=2.5 kV/m) with ε_{ms,avg}=0.57 kV/m (FIG. 26A) and 10.25 kV/m (FIG. 26B). ω=1.8 kHz.

FIG. 28 illustrates an analysis of the magnitude of the y-component of F_{DEP} in the xy-plane at z=5 μm. The scale bar indicates dielectrophoretic force (N).

FIG. 29A is a graph of electric field strength in the anodic channel above the BPE anode.

FIG. 29B is a graph of electric field strength in the anodic channel above the BPE cathode.

FIG. 29C illustrates controlled axial translation of the ion depletion zone.

FIG. 30 illustrates controlled lysis of a trapped cell.

### DETAILED DESCRIPTION

The present disclosure relates generally to methods, systems, and devices for dielectrophoretic manipulation of objects such as polarizable molecules and discrete polarizable solid, liquid, and mixed phases. In particular, the present disclosure relates to the use of actuating electrodes such as bipolar electrodes (BPEs) to generate and exert dielectrophoretic forces on objects within a fluidic device such as a microfluidic device. In certain aspects, the dielectrophoretic electric field can be shaped using localized control of the conductivity of the dielectrophoresis medium via faradaic ion enrichment and depletion at an array of BPEs. The advantages of BPEs for dielectrophoretic applications include their scalability and ability to impact an electric field through faradaic processes without direct electrical contact (e.g., conducting wires or other electrical connectors) between the BPE and an external instrument (e.g., a power source). In certain aspects, the methods, systems, and devices of the present disclosure provide: (1) strong electric field gradients (e.g., approximately 50 kV/m) without necessitating closely-spaced electrodes; (2) electric field gradients that extend further from the electrodes than those generated by traditional DEP electrodes, thus leading to the potential for higher throughput (e.g., trapping cells from a larger volume); and (3) trapping zones that can be fluidically mobilized.

The methods, systems, and devices of the present disclosure can be applied to the manipulation (e.g., transporting, sorting, trapping, filtering, etc.) of a wide variety of objects. Such objects can comprise, but are not limited to, chemicals, biochemicals, molecules (e.g., crystallizing molecules), genetic materials (e.g., DNA, RNA, and the like), expressed products of genetic materials, proteins, peptides, polypeptides, biological cells and compartments (e.g., eukaryotic cells, prokaryotic cells, organelles, exosomes, vesicles, liposomes), cellular fractions and lysates, viruses and viral particles, metabolites, drugs, particles (e.g., microparticles, microbeads, nanoparticles), nanotubes, and the like. In certain aspects, the object is a discrete phase (e.g., solid, liquid, or mixed phase) within a surrounding medium, such as a droplet, emulsion, or suspension. The object can be a polarizable object that develops an induced dipole moment when subjected to an electric field. In some aspects, the object is uncharged and/or is electrostatically neutral. In other aspects, the object possesses a net electrostatic charge, e.g., a net positive or net negative charge. Although certain aspects of the present disclosure are described in the context of manipulating cells, it shall be understood that the methods, systems, and devices of the present disclosure can be applied to any suitable object of interest.

Dielectrophoresis can be described as the generation of electrostatic force in the presence of a non-uniform electric field by the induction of an electrostatic dipole in an object (e.g., a molecule, particle, droplet, cell, etc.). Dielectrophoresis utilizes the attraction or repulsion of a polarizable object in a non-uniform electric field. Dielectrophoresis provides a versatile means of manipulating an object relative to a surrounding medium via the exertion of electrostatic force while not requiring that the phase possess a net electrostatic charge.

Advantageously, dielectrophoresis can be used to transport, sort, trap, and filter cells while maintaining a high degree of cell viability. The number of cells trapped is not purely statistically determined, but can be controlled by a number of experimental variables. The advantages of dielectrophoresis include: (1) distinguishing between cell types without the addition of labels or other expensive reagents (e.g., magnetic particles or fluorophores) owing to polarisabilities unique to cellular phenotype, size, and viability; (2) sufficiently inexpensive device components that allow for the production of disposable devices, an especially desirable characteristic for medical diagnostics devices for which cross-contamination must be avoided; (3) suitability for single cell manipulation, which can be achieved by constraining the trapping point (e.g., by adding physical barriers or by defining an electric field cage similar in size to a single cell) and/or selecting conditions that prevent cell-cell attraction in order to discourage multi-cell capture; and (4) simpler parallel operation compared to competing technologies such as optical tweezers or purely fluidic systems requiring a network of pumps and valves. However, existing dielectrophoresis technologies can be limited by difficulties in achieving arrays of local electric field gradients, limited ranges of electric field gradients, and fixed electric field gradient shapes.

In some aspects, the present disclosure provides methods, systems, and devices for the generation and manipulation of dielectrophoretic forces using faradaic (electron exchange) processes at an actuating electrode. An actuating electrode can comprise any semiconducting or conducting phase capable of facilitating faradaic charge transfer with a contacting medium (e.g., an ionically conductive phase). Faradaic processes at the actuating electrode can occur as a result of direct control of the electrical potential of the actuating electrode (e.g., via wire leads and a power supply) or as a result of an independently applied electric field (e.g., when the actuating electrode is a BPE as described below). Such faradaic processes are capable of causing gradients in a surrounding electric field. These gradients can result from localized alteration of ionic conductivity of the medium surrounding the actuating electrode and/or by virtue of the actuating electrode providing an alternate path for current (charge transport), as further described herein.

The actuating electrode and ionically conductive phase can be incorporated in a wide variety of fluidic devices. For example, the actuating electrode and ionically conductive phase can be contained in a fluidic channel can have openings (inlets and outlets) for the actuation of convective flow, introduction of objects, and application of the electric field. As another example, plurality of actuating electrodes arranged in an array format can be used to generate multiple charge enrichment and charge depletion zones through faradaic processes, as described further herein.

### Bipolar Electrochemistry

In some aspects of the present disclosure, the actuating electrode for generating dielectrophoretic forces is a BPE. Bipolar electrochemistry is a phenomenon defined by both anodic and cathodic faradaic reactions occurring simultaneously and in a coupled manner on a single conducting object (the BPE) that is electrically isolated from an external power source (i.e., no direct electrical connection). Contrary to a standard electrode, a BPE need not be in direct contact (e.g., physically touching or connected via wire leads) with the driving electrodes of a power source in order to facilitate faradaic reactions. In the presence of a sufficiently large electric field, the BPE facilitates coupled oxidation and reduction reactions at locations along the interface between the BPE and surrounding medium for which an electrical potential difference exists. For example, a BPE can comprise an electronic conductor (e.g., a strip of conductive material) in contact with an ionically conductive phase. The ionically conductive phase can comprise an aqueous solution containing ions (charged chemical species) capable of electromigration in the presence of an electric field, as described further herein. Driving electrodes in contact with the ionically conductive phase can apply the electric field. The electric field can comprise an alternating current (AC), direct current (DC), or a combination of AC and DC.

In certain aspects, when an electric field of sufficient magnitude is applied, faradaic processes occur at the BPE. The faradaic processes provide a path for current flow through the BPE in addition to or instead of the existing ionic current flowing in the ionically conductive phase. This alternate current path decreases ionic current in the competing path in the ionically conductive phase. This decrease results in a local minimum in electric field strength along that competing path. The gradient in electric field surrounding this local field minimum can exert dielectrophoretic forces on electrically polarizable species (e.g., molecules, particles, droplets, cells, etc.) within the ionically conductive phase, also referred to herein as “discrete phases.” Specifically, polarizable discrete phases can be accelerated towards (negative dielectrophoresis, nDEP) or away from (positive dielectrophoresis, pDEP) this electric field minimum. Acceleration by dielectrophoretic force can be linear or angular, resulting in attraction, repulsion, trapping, curved trajectory, rotation, and increase or decrease in velocity.

The BPEs of the present disclosure can be fabricated in a variety of ways. For example, a BPE can be fabricated from a single material or from a combination of multiple different materials. In some aspects, the BPE is fabricated from conductive materials. Exemplary conductive materials include, but are not limited to, conductive metals (e.g., Sn, Zn, Au, Ag, Ni, Pt, Pd, Al, In, Cu, or combinations thereof), metal oxides, and/or conductive non-metals (e.g., conducting polymers). A BPE can be provided as a strip, wire, film, coating, and the like. BPEs can be fabricated using any of many approaches including, but not limited to, photolithographic patterning (e.g., lift-off lithography, dry etch, or wet etch), screen printing, machining, soft lithography, or electroplating, or combinations thereof.

The dimensions of the BPEs of the present disclosure can be varied as desired. For example, in some aspects, the BPE is approximately coplanar with a surface supporting the BPE (e.g., a floor of a fluidic containment structure). In other aspects, a BPE can have a height that is less than about 5 μm, less than about 4 μm, less than about 3 μm, less than about 2 μm, less than about 1 μm, less than about 900 nm, less than about 800 nm, less than about 750 nm, less than about 700 nm, less than about 600 nm, less than about 500 nm, less than about 400 nm, less than about 300 nm, less than about 250 nm, less than about 200 nm, or less than about 100 nm. The length of the BPE (e.g., the length of the surface in electrochemical contact with the ionically conductive phase) can be at least about 1 μm, at least about 5 μm, at least about 10 μm, at least about 50 μm, at least about 100 μm, at least about 200 μm, at least about 250 μm, at least about 300 μm, at least about 400 μm, at least about 500 μm, at least about 600 μm, at least about 700 μm, at least about 800 μm, at least about 900 μm, or at least about 1 mm. The width of the BPE can be at least about 1 μm, at least about 5 μm, at least about 10 μm, at least about 50 μm, at least about 100 μm, at least about 200 μm, at least about 250 μm, at least about 300 μm, at least about 400 μm, at least about 500 μm, at least about 600 μm, at least about 700 μm, at least about 800 μm, at least about 900 μm, or at least about 1 mm.

In some aspects, the BPEs described herein are in electrical communication with an ionically conductive phase capable of facilitating the electrochemical reactions described herein. For example, a BPE or at least a portion thereof (e.g., an end portion or tip of the BPE) can be immersed within or otherwise in direct contact with an ionically conductive phase. A wide variety of fluids and liquids can be used for the ionically conductive phase. In some aspects, the ionically conductive phase includes an aqueous solution containing ions capable of electromigration in the presence of an electric field. Possible aqueous solutions that can be used for the ionically conductive phase include water-based solutions that can further include buffers, salts, and other components generally known to be used in dielectrophoresis. In some aspects, the ionically conductive phase can also include analysis reagents as described further herein. The ionically conductive phase can be stationary or can be mobile (e.g., relative to the BPE). For example, the ionically conductive phase can be flowed by various approaches including, but not limited to, gravity, air pressure, syringe pump, peristaltic pump, electroosmotic flow, application of vacuum, or suitable combinations thereof. Any suitable mechanism for actuating flow of the ionically conductive phase can be incorporated within or used in conjunction with the systems and devices of the present disclosure.

In some aspects, an electric field is applied to the ionically conductive phase in order to produce the electrochemical reactions described herein. The electric field can comprise only an AC component or only a DC component, or the applied field can comprise a combination of both AC and DC components. These AC and/or DC components can be applied for varying lengths of time and at constant or changing magnitudes.

The AC component can be varied based on the specific type of BPE, ionically conductive phase, and/or object to be manipulated, as well as based on the specific geometry and configuration of the dielectrophoretic device. For example, for dielectrophoretic manipulation of cells, the minimum spatially averaged root-mean-square (RMS) AC electric field (Eᵣₘₛ) can be approximately 10 kV/m and the maximum spatially averaged RMS AC electric field (Eᵣₘₛ) can be approximately 100 kV/m. In some aspects, field strengths below this range will not provide relevant dielectrophoretic force for manipulation of eukaryotic cells (having diameters in tens of microns). Conversely, higher field strengths can be employed to electroporate or lyse cells.

In certain aspects, the upper limit for the frequency of the AC component is approximately 1 GHz, while the lower limit for the frequency is approximately 1 Hz. In order to increase device longevity, the low frequency limit can be further bounded to prevent significant AC faradaic current, which can contribute to degradation of the electrode material. The AC faradaic current can be limited by setting the field frequency faster than the rate of electron transfer (i.e., the faradaic reaction rate). The boundary is defined by the relation ω≧20/D, where ω is the angular frequency of the applied AC electric field, k° is the standard rate constant for the heterogeneous reaction (faradaic reaction) employed, and D is the diffusion coefficient of the electroactive species (reagent). For example, in the case that D=1×10⁻⁵ cm²/s and k°=0.01 cm/s (a moderate reaction rate), ω can be greater than 20 Hz. Similarly, for a fast reaction (k°=0.1 cm/s), ω can be greater than 2 kHz.

In some aspects, the electric field comprises an AC component having a frequency of about 1 kHz, about 10 kHz, about 50 kHz, about 100 kHz, about 500 kHz, about 1000 kHz, about 5000 kHz, about 10,000 kHz, about 50,000 kHz, about 0.1 MHz, about 0.5 MHz, about 1 MHz, about 5 MHz, about 10 MHz, or about 50 MHz. In certain aspects, the AC component has a frequency range of up to about 1 GHz, such as from about 1 kHz to about 100 MHz. The peak-to-peak amplitude of the AC component can be about 1 mV, about 5 mV, about 10 mV, about 50 mV, about 100 mV, about 500 mV, about 1 V, about 5 V, about 10 V, about 50 V, about 100 V, or about 1 kV. For example, the AC component can have a voltage range from about 1 V to about 1 kV. In some aspects, the electric field strength of the AC component is about 10 kV/m, about 50 kV/m, about 100 kV/m, about 500 kV/m, about 1000 kV/m, about 1 MV/m, about 5 MV/m, about 10 MV/m, about 50 MV/m, about 100 MV/m or about 500 MV/m. The AC electric field strength can be varied within a range based on the type of object to be manipulated, such as from about 10 kV/m to about 1000 kV/m (e.g., for biological cells, microbeads), from about 100 kV/m to about 1000 kV/m (e.g., for conductive nanotubes), from about 1000 kV/m to about 10 MV/m (e.g., for nanoparticles), or from about 1 MV/m to about 100 MV/m (e.g., for biomolecules such as DNA or large proteins, viral particles).

Similarly, the DC component can be varied based on the specific type of BPE, ionically conductive phase, and/or object to be manipulated, as well as based on the specific geometry and configuration of the dielectrophoretic device. The range of relevant DC field strengths is determined by the geometry of the device, the identity of the electroactive species available for faradaic reactions at the BPE, and the electrochemical properties of the BPE material employed.

In some aspects, the DC field strength minimum satisfies two conditions. First, ΔU_{BPE}≧|U°_{red}−U°ₒₓ|, where ΔU_{BPE} is the total potential available to drive faradaic reactions at the bipolar electrode (BPE) and U°_{red} and U°ₒₓ are the standard reduction potentials for the cathodic (reduction) and anodic (oxidation) reactions, respectively, employed at the BPE. ΔU_{BPE} is defined as the difference of the maximum (most positive) and minimum electrical potentials of the solution phase in contact with the BPE cathode and anode, respectively (i.e., ΔU_{BPE}=U_{cathode}−U_{anode}). Second, there is sufficient overpotential to drive faradaic reactions at the BPE such that i_{BPE}≠0. This condition is distinct from the first in that a number of experimental factors (e.g., non-ideal electrode material) may prevent faradaic reactions from occurring at their standard potentials. In these cases, a higher overpotential can be used.

In some aspects, the DC field strength maximum satisfies two conditions. First, ΔU_{BPE} is sufficiently low to avoid electrode damage. For example, if an Au BPE is employed, U°ₒₓ for Au oxidation is +1.5 V versus the Standard Hydrogen Electrode (SHE). If there is a chemical species available for reduction at the BPE at U°_{red}=−1.5 V versus SHE, then ΔU_{BPE} can be constrained so as to not exceed 3.0 V. Significantly, the electrode material and other experimental variables greatly impact the actual value of ΔU_{BPE} at which electrode damage occurs. For example, in the presence of Cl—, Au oxidation proceeds at a much lower potential (U°ₒₓ is +1.0 V versus SHE). Second, ΔU_{BPE} is sufficiently low to avoid formation of gases exceeding the solvating capacity of the aqueous medium. For example, ΔU_{BPE} above approximately 3.0 V, may drive water electrolysis at a sufficient rate to produce O₂ and H₂ gas bubbles at the BPE anode and cathode, respectively.

In some aspects, the electric field comprises a DC component having a positive or negative sign and a magnitude of about 0 V, about 1 mV, about 5 mV, about 10 mV, about 50 mV, about 100 mV, about 500 mV, about 1 V, about 5 V, about 10 V, about 50 V, about 100 V, about 1 kV, or about 5 kV. In certain aspects, the DC component has a voltage range from about 10 mV to 100 V.

The electric field can be applied in a wide variety of ways. In some aspects, driving electrodes connected to a power source are used to apply the electrical field. The power source can be any system or device capable of applying the electric field having the AC and/or DC components at the selected frequency and voltage. The power source may comprise a waveform generator and, if desired, a bipolar operational amplifier having an appropriate bandwidth for the selected frequency. For example, the power source can comprise a waveform generator capable of outputting 10 V peak-to-peak and an amplifier capable of 1000 V bipolar output. The structure of the driving electrodes can be varied as desired, including wires, rods, plates, comb-like structures, and the like. In some aspects, when more than two driving electrodes are used, one or more of the driving electrodes can be allowed to float (i.e., having an electrical potential that is not externally controlled).

Fluidic devices incorporating BPEs

In some aspects, the BPE and ionically conductive phase are incorporated within a fluidic device. Such fluidic devices can comprise various fluid containment structures adapted to contain or transport fluids, such as channels, chambers, ports, tubes, wells, or combinations thereof. Certain aspects of the present disclosure are suitable for use in small scale fluidic devices, such as microfluidic devices.

In certain aspects, the BPE or a portion thereof is situated in a fluidic containment structure comprising an ionically conductive phase. A fluidic containment structure can be defined by one or more defining surfaces (e.g., walls, ceiling, floor) that enclose the interior volume of the containment structure. In certain aspects, the defining surfaces can be made of one or more of glass, plastics, polycarbonate, polyurethanemethacrylate (PUMA), cyclic olefin copolymer (COC), polymethylmethacrylate (PMMA), polydimethylsiloxane (PDMS) and other insulating materials (e.g., electrically insulating materials). In some aspects, the defining surfaces can be made of porous material (e.g., polycarbonate membrane, hydrogel materials, ionically-conductive polymers, etc.). The fluidic containment structures can be fabricated using various approaches including, but not limited to, photolithographic patterning of a photoresist, mold and caste, injection molding, hot embossing, micromachining, wet etching, dry etching (e.g., deep reactive ion etch), or suitable combinations thereof. For mold and caste, the mold can be fabricated by any of the same approaches as the channels. In some aspects, a fluidic containment structure can have openings (inlets and outlets) for the actuation of convective flow, introduction of discrete phases, and application of the electric field. Inlets and outlets to the fluidic containment structure can be included during fabrication processes or can be introduced at a later timepoint via fabrication methods such as drilling, etching, or punching (e.g., die cut).

The dimensions of the fluidic containment structures described herein can be varied as desired. For example, the width of a fluidic containment structure can be at least about 1 μm, at least about 5 μm, at least about 10 μm, at least about 50 μm, at least about 100 μm, at least about 200 μm, at least about 250 μm, at least about 300 μm, at least about 400 μm, at least about 500 μm, at least about 600 μm, at least about 700 μm, at least about 800 μm, at least about 900 μm, or at least about 1 mm. In some aspects, the width of the fluidic containment structure is less than or equal to the width of the BPE. In other aspects, the width of the fluidic containment structure is greater than or equal to the width of the BPE. The length of a fluidic containment structure can be can be at least about 1 μm, at least about 5 μm, at least about 10 μm, at least about 50 μm, at least about 100 μm, at least about 200 μm, at least about 250 μm, at least about 300 μm, at least about 400 μm, at least about 500 μm, at least about 600 μm, at least about 700 μm, at least about 800 μm, at least about 900 μm, at least about 1 μm, at least about 2 mm, at least about 3 mm, at least about 4 mm, at least about 5 mm, at least about 6 mm, at least about 7 mm, at least about 8, mm, at least about 9 mm, at least about 10 mm, at least about 5 cm, at least about 10 cm, at least about 20 cm, at least about 30 cm, at least about 40 cm, at least about 50 cm, at least about 60 cm, at least about 70 cm, at least about 80 cm, at least about 90 cm, or at least about 100 cm. The height of a channel can be can be at least about 100 nm, at least about 200 nm, at least about 300 nm, at least about 400 nm, at least about 500 nm, at least about 600 nm, at least about 700 nm, at least about 800 nm, at least about 900 nm, at least about 1 μm, at least about 5 μm, at least about 10 μm, at least about 50 μm, at least about 100 μm, at least about 200 μm, at least about 500 μm, or at least about 1 mm.

The devices of the present disclosure can be fabricated in a variety of ways. In some aspects, defining surfaces and BPEs can be aligned and bonded. Bonding can be achieved through many approaches including, but not limited to, thermal bonding (e.g., welding or fusing), exposure to an oxygen or nitrogen plasma, chemical surface modification for covalent bonding (e.g., bi-functional silane reagents), fixatives (e.g., glue, epoxy, adhesive tapes or films), light or UV assisted bonding, reversible conformal contact bonding, or suitable combinations thereof.

In some aspects, the fluidic containment structure comprises a fluidic channel. The geometry of a fluidic channel can be defined by channel-defining surfaces (e.g., walls, floor, ceiling). For example, a channel can be linear, curved, or curvilinear. The cross-sectional shape of the channel can be varied as desired, e.g., square, rectangular, or circular. In certain aspects, the length of the channel is greater than its width and/or height.

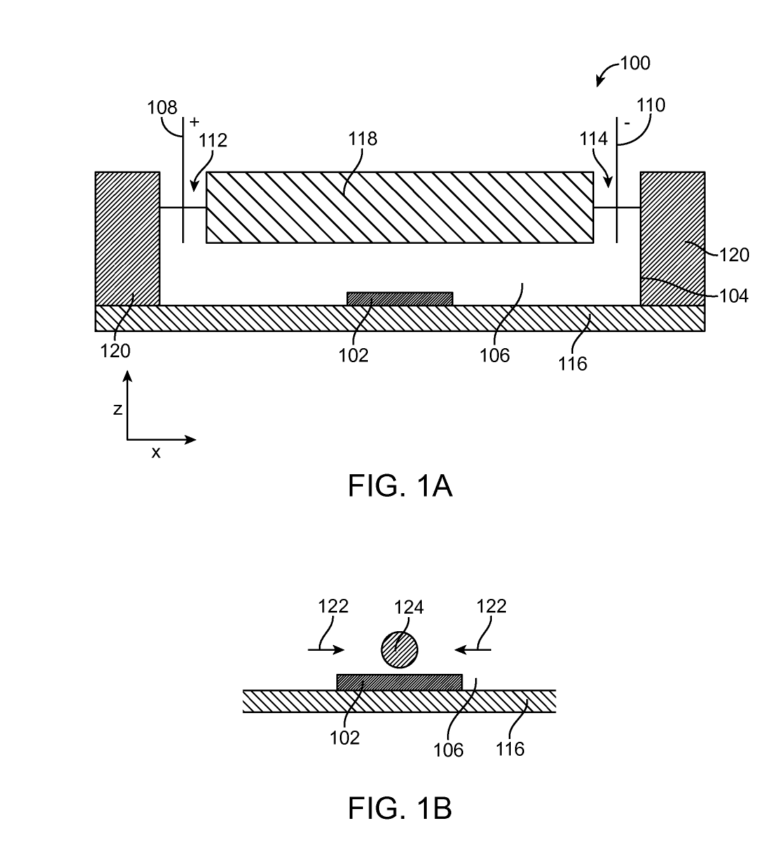

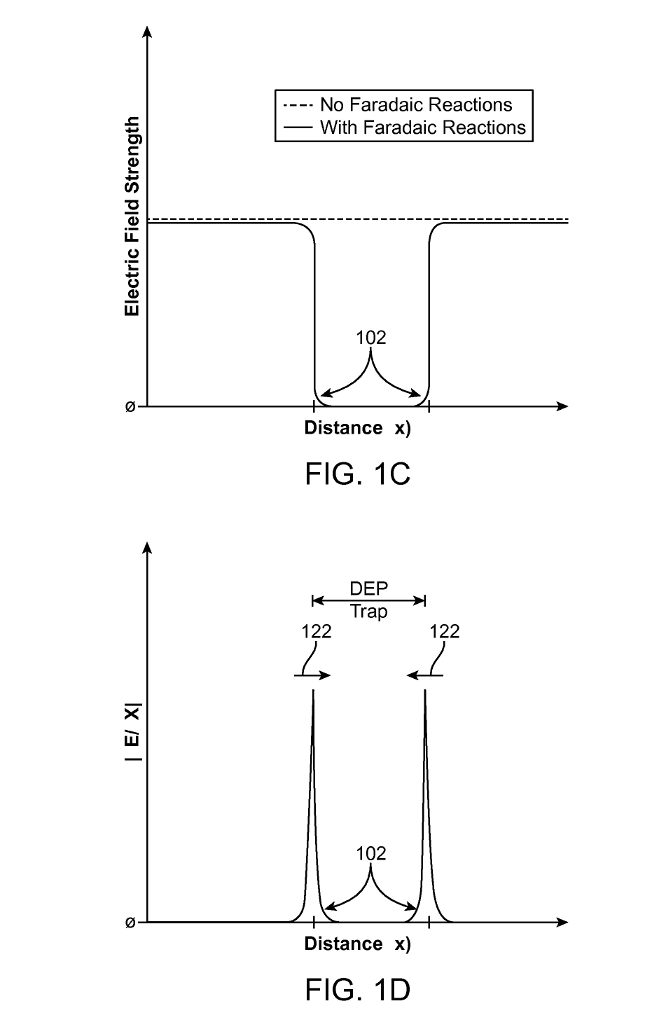

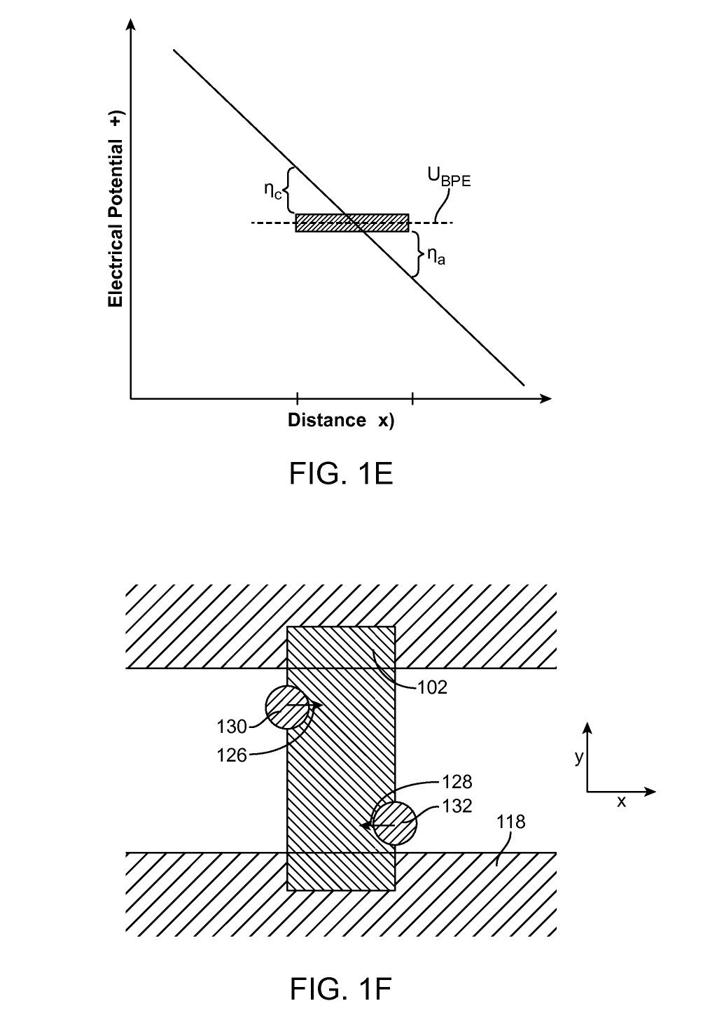

FIG. 1A is a cross-sectional view of a single channel BPE device 100. The device 100 comprises a single BPE 102 (e.g., an Au electrode) in a fluidic channel 104 filled with an ionically conductive phase 106 (e.g., an aqueous solution). Driving electrodes 108, 110 in contact with the ionically conductive phase 106 apply a voltage bias. The ionically conductive phase 106 and/or polarizable discrete phases (e.g., cells) can be added via reservoirs (not shown). In some aspects, the reservoirs are in fluid communication with the channel 104 via apertures 112, 114. The channel-defining surfaces of the device 100 (e.g., floor 116, ceiling 118, and walls 120) can be fabricated from any suitable material. For example, the floor 116 can be fabricated from glass, while the ceiling 118 and walls 120 can be fabricated from PDMS. The driving electrodes (108 and 110) can be comprised of thin film conductive materials attached to a channel-defining surface or comprised of conductive wire inserted into the ionically conductive phase 106.

The BPE 102 can be coplanar with the channel floor 116 or have a height that can be less than about 5 μm, less than about 4 μm, less than about 3 μm, less than about 2 μm, less than about 1 μm, less than about 900 nm, less than about 800 nm, less than about 750 nm, less than about 700 nm, less than about 600 nm, less than about 500 nm, less than about 400 nm, less than about 300 nm, less than about 250 nm, less than about 200 nm, or less than about 100 nm. The length of the BPE 102 surface in electrochemical contact with the ionically conductive phase 106 can be at least about 1 μm, at least about 5 μm, at least about 10 μm, at least about 50 μm, at least about 100 μm, at least about 200 μm, at least about 250 μm, at least about 300 μm, at least about 400 μm, at least about 500 μm, at least about 600 μm, at least about 700 μm, at least about 800 μm, at least about 900 μm, or at least about 1 mm. The width of the BPE 102 can be greater than or equal to the width of the channel 104, or the width of the BPE 102 can be less than the width of the channel 104. Specifically, the width of the BPE 102 can be at least about 1 μm, at least about 5 μm, at least about 10 μm, at least about 50 μm, at least about 100 μm, at least about 200 μm, at least about 250 μm, at least about 300 μm, at least about 400 μm, at least about 500 μm, at least about 600 μm, at least about 700 μm, at least about 800 μm, at least about 900 μm, or at least about 1 mm.

The width of the channel 104 can be at least about 1 μm, at least about 5 μm, at least about 10 μm, at least about 50 μm, at least about 100 μm, at least about 200 μm, at least about 250 μm, at least about 300 μm, at least about 400 μm, at least about 500 μm, at least about 600 μm, at least about 700 μm, at least about 800 μm, at least about 900 μm, or at least about 1 mm. The length of the channel 104 can be can be at least about 1 μm, at least about 5 μm, at least about 10 μm, at least about 50 μm, at least about 100 μm, at least about 200 μm, at least about 250 μm, at least about 300 μm, at least about 400 μm, at least about 500 μm, at least about 600 μm, at least about 700 μm, at least about 800 μm, at least about 900 μm, at least about 1 mm, at least about 2 mm, at least about 3 mm, at least about 4 mm, at least about 5 mm, at least about 6 mm, at least about 7 mm, at least about 8, mm, at least about 9 mm, at least about 10 mm, at least about 5 cm, at least about 10 cm, at least about 20 cm, at least about 30 cm, at least about 40 cm, at least about 50 cm, at least about 60 cm, at least about 70 cm, at least about 80 cm, at least about 90 cm, or at least about 100 cm. The height of the channel 104 can be can be at least about 100 nm, at least about 200 nm, at least about 300 nm, at least about 400 nm, at least about 500 nm, at least about 600 nm, at least about 700 nm, at least about 800 nm, at least about 900 nm, at least about 1 μm, at least about 5 μm, at least about 10 μm, at least about 50 μm, at least about 100 μm, at least about 200 μm, at least about 500 μm, or at least about 1 mm.

FIG. 1B illustrates a limited view of the device 100 depicted in FIG. 1A, in which nDEP force 122 acts on a polarizable discrete phase 124 to trap it at the BPE 102. FIG. 1C shows the electric field strength in a segment of the ionically conductive phase 106 surrounding the BPE 102 in the presence (solid line) and absence (dashed line) of faradaic reactions. There is a local field minimum over the BPE 102, which can serve as a trapping location for a polarizable discrete phase under appropriate conditions (e.g., electric field frequency, conductivity of ionically conductive phase, polarizability of discrete phase to be trapped) for nDEP. FIG. 1D illustrates the relative magnitude of the electric field gradient, shown as the absolute value of the derivative of the electric field strength in the x-direction. The nDEP force 122 points inward on both sides of the BPE 102.

FIG. 1E illustrates the electrical potential in the ionically conductive phase 106 in a segment of the channel surrounding the BPE 102. In certain aspects, a linear potential profile develops when a DC voltage bias is applied across the fluidic channel 104 by the electrodes. The potential of the BPE 102 (U_{BPE}) floats to a value intermediate to the potential of the ionically conductive solution 106 in contact with its ends. The cathodic (η_{c}) and anodic (ηₐ) overpotentials result from a difference between the electrical potential of the BPE 102 (U_{BPE}) and that of the ionically conductive phase 106. A sufficiently large overpotential can drive faradaic (electron transfer) reactions between the BPE 102 and chemical species in the ionically conductive phase 106. In some aspects, the potential difference (η) between the BPE 102 and ionically conductive phase 106 is a driving force for oxidation (ηₐ) and reduction (η_{c}) reactions at opposite ends of the BPE 102. The cathodic and anodic reactions can be coupled by the BPE 102 such that an equal number of electrons are accepted and donated by the BPE 102. Significantly, faradaic reactions can be achieved at the BPE 102 without requiring direct electrical contact between the electrodes and the BPE 102. This feature allows multiple BPEs to be operated in parallel, as described further herein. The rates of electron transfer to (oxidation) and from (reduction) the BPE 102 are coupled and lead to a current through the BPE (i_{BPE}). Note that when i_{BPE} is non-zero, it competes with ionic current in the fluidic channel 104 and impacts the potential drop in the ionically conductive phase 106 as indicated by the dashed line in FIG. 1E.

FIG. 1F shows a top view of a limited portion of the device 100 depicted in FIG. 1A. Specifically, FIG. 1F shows a top view of the BPE 102 spanning the fluidic channel 104. The arrows 126, 128 depict the nDEP force on discrete polarizable phases 130, 132 as they approach the BPE 102. This attractive force is the basis for the trapping mechanism.

The devices of the present disclosure can incorporate any suitable number and combination of BPEs. For example, a device can include one, two, three, four, five, six, seven, eight, nine, ten, or more BPEs. In certain aspects, a device can include at least ten BPEs, at least 20 BPEs, at least 30 BPEs, at least 40 BPEs, at least 50 BPEs, at least 60 BPEs, at least 70 BPEs, at least 80 BPEs, at least 90 BPEs, at least 100 BPEs, at least 200 BPEs, at least 300 BPEs, at least 400 BPEs, at least 500 BPEs, at least 600 BPEs, at least 700 BPEs, at least 800 BPEs, at least 900 BPEs, at least 1000 BPEs, at least 2000 BPEs, at least 3000 BPEs, at least 4000 BPEs, or at least 5000 BPEs. The BPEs can be of the same or a similar type (e.g., with respect to composition, geometry, size, etc.). Alternatively, at least some of the BPEs can be of a different type than other BPEs. The BPEs can be arranged in any suitable configuration, such as a two-dimensional (2D) array or a three-dimensional (3D) array. An array of BPEs can comprise a plurality of BPEs arranged in a repeating pattern, such as a pattern comprising a plurality of rows and columns.

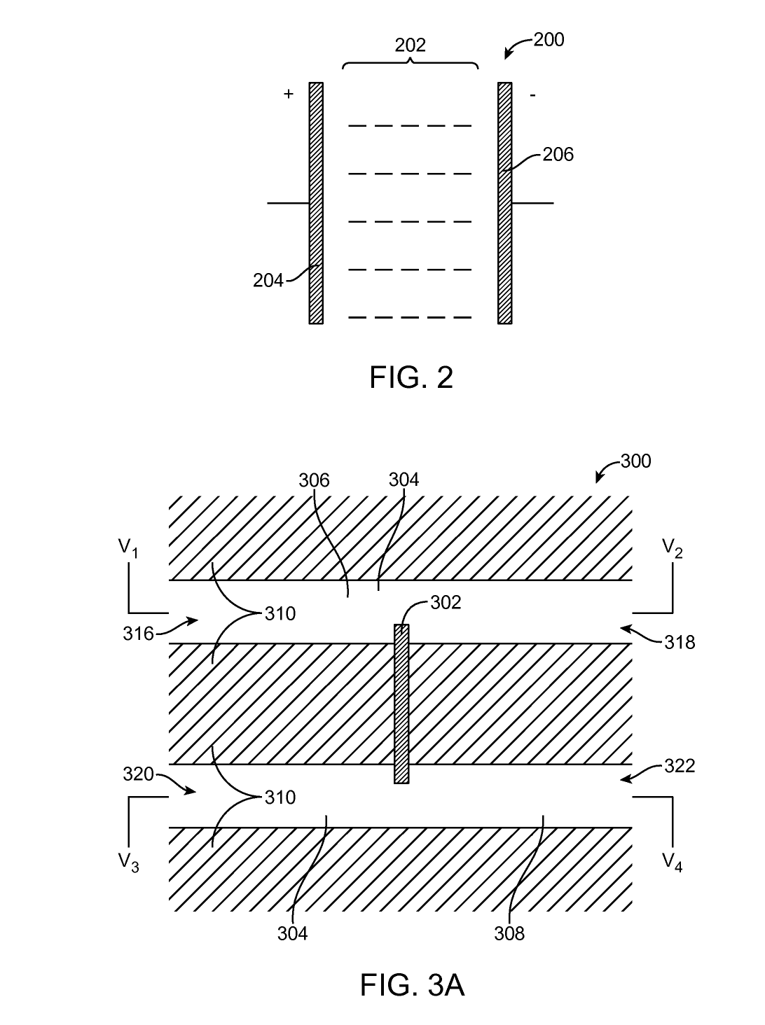

FIG. 2 illustrates a device 200 in which multiple BPEs 202 are arranged on an insulating substrate in an array format. The device 200 can be at least partially immersed in an ionically conductive phase such that each of the BPEs 202 is in electrical communication with the ionically conductive phase. These BPEs 202 can generate a plurality of local electric field minima. In some aspects, the device 200 can comprise a 2D array or a 3D array. A 2D array is illustrated in FIG. 2, in which driving electrodes 204, 206 can be located on either side of an array of BPEs 202. The driving electrodes 204, 206 can be comprised of a thin film of conductive material or comprised of conductive plates. The driving electrode dimensions can be determined by the size and shape of the BPE array such that the driving electrodes 204, 206 can provide the desired electric field strength at each point of the array. This electric field strength can be uniform or non-uniform, and to cause manipulation (e.g., trapping), the field can be strong enough to drive electrochemical reactions at the BPEs 202.

In reference to FIG. 2, each of the BPEs 202 can be coplanar with the substrate or have a height that is less than about 1 μm, less than about 900 nm, less than about 800 nm, less than about 750 nm, less than about 700 nm, less than about 600 nm, less than about 500 nm, less than about 400 nm, less than about 300 nm, less than about 250 nm, less than about 200 nm, or less than about 100 nm. The length of each BPE surface in electrochemical contact with the ionically conductive phase can be at least about 1 μm, at least about 5 μm, at least about 10 μm, at least about 50 μm, at least about 100 μm, at least about 200 μm, at least about 250 μm, at least about 300 μm, at least about 400 μm, at least about 500 μm, at least about 600 μm, at least about 700 μm, at least about 800 μm, at least about 900 μm, or at least about 1 mm. The width of each BPE can be at least about 1 μm, at least about 5 μm, at least about 10 μm, at least about 50 μm, at least about 100 μm, at least about 200 μm, at least about 250 μm, at least about 300 μm, at least about 400 μm, at least about 500 μm, at least about 600 μm, at least about 700 μm, at least about 800 μm, at least about 900 μm, or at least about 1 mm.

In certain aspects of the present disclosure, a device can include a plurality of fluidic channels each in contact with a BPE or a portion thereof. For example, a device can include one, two three, four, five, six, seven, eight, nine, ten, or more channels each in contact with at least a portion of a BPE. In some aspects, at least some or all of the fluidic channels are fluidically isolated from each other. Fluidically isolated channels can each contain the same ionically conductive phase, or can contain different ionically conductive phases. In other aspects, at least some or all of the fluidic channels are in fluid communication with each other. The arrangement and geometry of the fluidic channels can be varied as desired. For example, channels can be arranged in a parallel configuration, perpendicular configuration, intersecting configuration, branching configuration, or suitable combinations thereof.

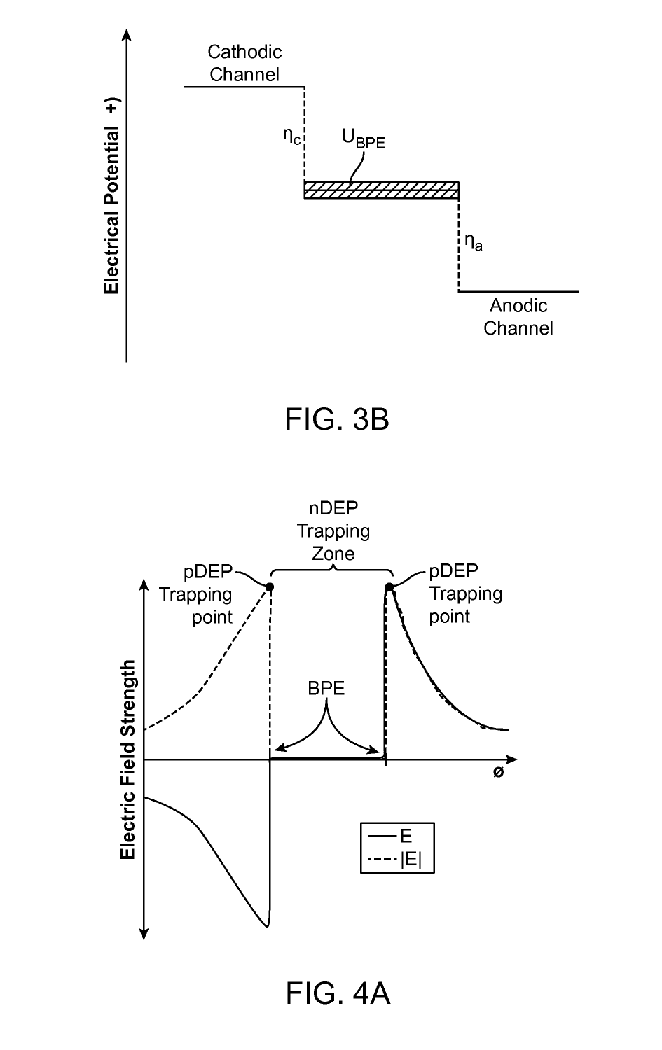

FIG. 3A illustrates a dual-channel device 300 in which a BPE 302 contacts an ionically conductive phase 304 in two channels 306, 308. The two channels 306, 308 are fluidically isolated from each other by channel boundaries 310. In some aspects, the channel boundaries 310 are comprised of insulating material, e.g., in order to electrically insulate the channels 306, 308 from each other. The BPE 302 extends across the boundary 310 between the two channels 306, 308 such that one end portion 312 of the BPE 302 contacts the ionically conductive phase 304 contained within the channel 306 and the opposite end portion 314 contacts the ionically conductive phase 304 contained within the channel 308. In some aspects, only the end portions 312, 314 of the BPE 302 that extend past the boundary 310 are in electrochemical contact with the ionically conductive phase 304, while the central portion of the BPE 302 spanning the boundary 310 is not in electrochemical contact with the ionically conductive phase 304.

In certain aspects, the device 300 includes four inlets 316, 318, 320, 322 where voltage can be applied (V₁, V₂, V₃, and V₄). A voltage bias can be applied across the two channels 306, 308 such that coupled oxidation and reduction reactions occur at separate ends of the BPE 302 in contact with the ionically conductive phase in each channel. FIG. 3B illustrates the electrical potential of the ionically conductive phase 304 at the cathodic and anodic ends of the BPE 302 and the electrical potential of the BPE 302. Specifically, in one channel (the cathodic channel, e.g., channel 306), the electrical potential of the ionically conductive phase in contact with the BPE 302 can be higher (more positive) than the electrical potential of the BPE 302, leading to electron transfer from the BPE 302 to chemical species in the ionically conductive phase 304 (electrochemical reduction). In the other channel (the anodic channel, e.g., channel 308), the electrical potential of the ionically conductive phase 304 in contact with the BPE 302 can be lower (more negative) than the electrical potential of the BPE 302, leading to electron transfer from chemical species in the ionically conductive phase to the BPE 302 (electrochemical oxidation).

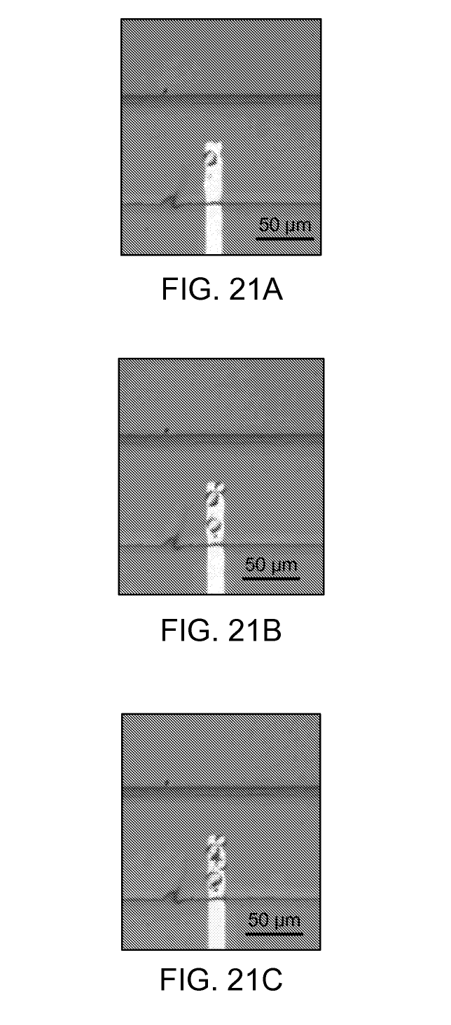

The geometry and dimensions of the device 300 can be varied as desired. In some aspects, the BPE 302 is rectangular, as depicted in FIG. 3A. In other aspects, the BPE 302 need not be a rectangle, but can also be implemented with tapered, pointed, rounded, split, ring, or other shaped tip. In some aspects, the entire BPE 302 can be a rectangle, circle, triangle, ellipse, or open shape (e.g., a ring). An example of a BPE with a tapered tip is shown in FIG. 21A. In certain aspects of the present disclosure, the BPE 302 and channels 306, 308 can have similar dimensions to the BPE 102 and channel 104, respectively, described in the device 100 depicted in FIG. 1A. The channels 306, 308 can have equal or unequal dimensions compared to each other. For example, the width of the channel 306 can be greater than the width of the channel 308, or vice-versa. Additionally, the BPE 302 can have a greater length than the BPE 102 described in FIG. 1A. The BPE 302 can be sufficiently long so as to be in electrochemical contact with the two channels 306, 308. The BPE 302 can also span the two channels 306, 308 such that its length exceeds the distance between the outermost walls of the channels 306, 308. Specifically, the BPE 302 can have a length of at least about 5 μm, at least about 10 μm, at least about 50 μm, at least about 100 μm, at least about 200 μm, at least about 250 μm, at least about 300 μm, at least about 400 μm, at least about 500 μm, at least about 600 μm, at least about 700 μm, at least about 800 μm, at least about 900 μm, at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, at least about 15 mm, at least about 20 mm, at least about 30 mm, at least about 40 mm, or at least about 50 mm. The distance between the channels 306, 308 can be chosen such that the BPE length is sufficient to remain in electrochemical contact with the two channels 306, 308.

### Formation of Electric Field Minima and Maxima Via Faradaic Processes

In various aspects of the present disclosure, the electric field can be applied with sign and magnitude such that the ionically conductive phase contacting either end or both ends of the BPE is at a local electric field minimum. Specifically, the sign of the electric field can transition from negative to positive or from positive to negative in the segment of the ionically conducting phase in contact with the BPE such that the electric field is zero in magnitude in this segment.

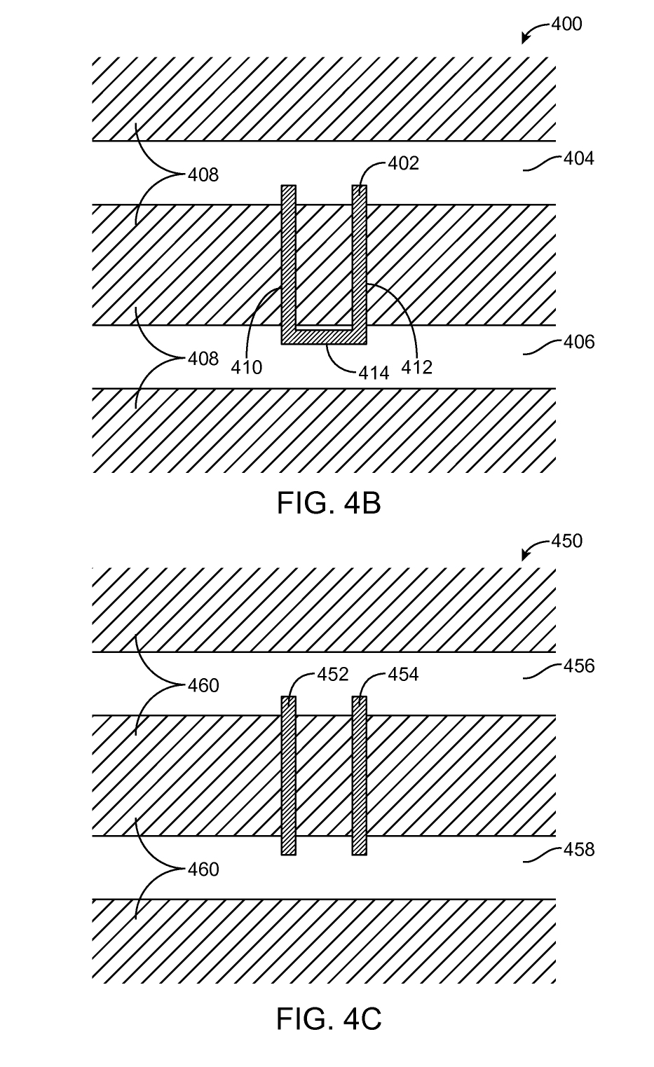

FIG. 4A illustrates such an electric field profile in a segment of the ionically conductive phase surrounding a BPE. The electric field profile comprises pDEP trapping points positioned at either end of a nDEP trapping zone in the ionically conductive phase. The pDEP trapping points correspond to the ends of the BPE while the nDPE trapping zone corresponds to the length of the BPE. The gradient in electric field surrounding this local field minimum can exert dielectrophoretic force on electrically polarizable discrete phases (e.g., molecules, particles, droplets, etc.). Specifically, polarizable discrete phases can be accelerated towards (nDEP) or away from (pDEP) this electric field minimum.

In some aspects, two or more BPE portions can be positioned relative to each other such that the electric field in the segment of the ionically conductive phase located between the BPE portions is the same or similar to the electric field at the BPE portions (e.g., the electric field between the BPE portions is also be at a local electric field minimum). The spacing between the BPE portions can be varied as necessary to achieve this effect. For example, the BPEs can be spaced less than or equal to about 1 μm to 500 μm apart. The BPE portions can be portions of a single BPE, portions of different BPEs, or suitable combinations thereof. For example, in certain aspects, two or more BPEs traverse the two channels of a dual-channel device such that the region of the ionically conductive phase in the channels between the outermost BPEs can have an electric field that is zero in magnitude.

FIG. 4B illustrates a device 400 including a single BPE 402 and two channels 404, 406 surrounded and defined by an insulating material 408. In some aspects, the BPE 402 is U-shaped and includes a first end portion 410, second end portion 412, and a central portion 414 joining the first and second end portions 410, 412. The angles between the first end portion 410, second end portion 412, and the central portion 414 can be varied as desired. For example, in certain aspects, the first and second end portions 412, 414 are parallel or approximately parallel to each other and the central portion 414 is perpendicular or approximately perpendicular to the first and second end portions 412, 414. The first and second end portions 410, 412 are each in contact with each of the two channels 404, 406, while the central portion 414 is in contact with only a single channel 406.

FIG. 4C illustrates a similar device 450 in which two BPEs 452, 454 are each in electrochemical contact with each of two channels 456, 458 defined by an insulating material 460. The BPEs 452 and 454 can be arranged at any suitable angle relative to each other. For instance, the BPEs 452 and 454 can be parallel or approximately parallel to each other. Each of the two BPEs 452, 454 are in contact with each of the channels 456, 458. The BPE(s) and channels of FIGS. 4B and 4C can have similar dimensions to the BPE and channels, respectively, described with respect to FIG. 3A. The device 400 of FIG. 4B and device 450 of FIG. 4C can be used to increase the volume of the region in which objects such as polarizable discrete phases can be manipulated (e.g., attracted and trapped, repelled and excluded).

In another aspect of the present disclosure, a plurality of channels and BPEs (e.g., as depicted in FIGS. 3A, 4B, and 4C), can be employed to generate an array of electric field minima for dielectrophoretic attraction or repulsion of polarizable discrete phases, with each electric field minimum associated with an end of a BPE as described herein. Notably, the present disclosure enables generation of an array of electric field minima without necessitating direct contact (e.g., via electrical connections such as wires) between the driving electrodes and each of the BPEs.

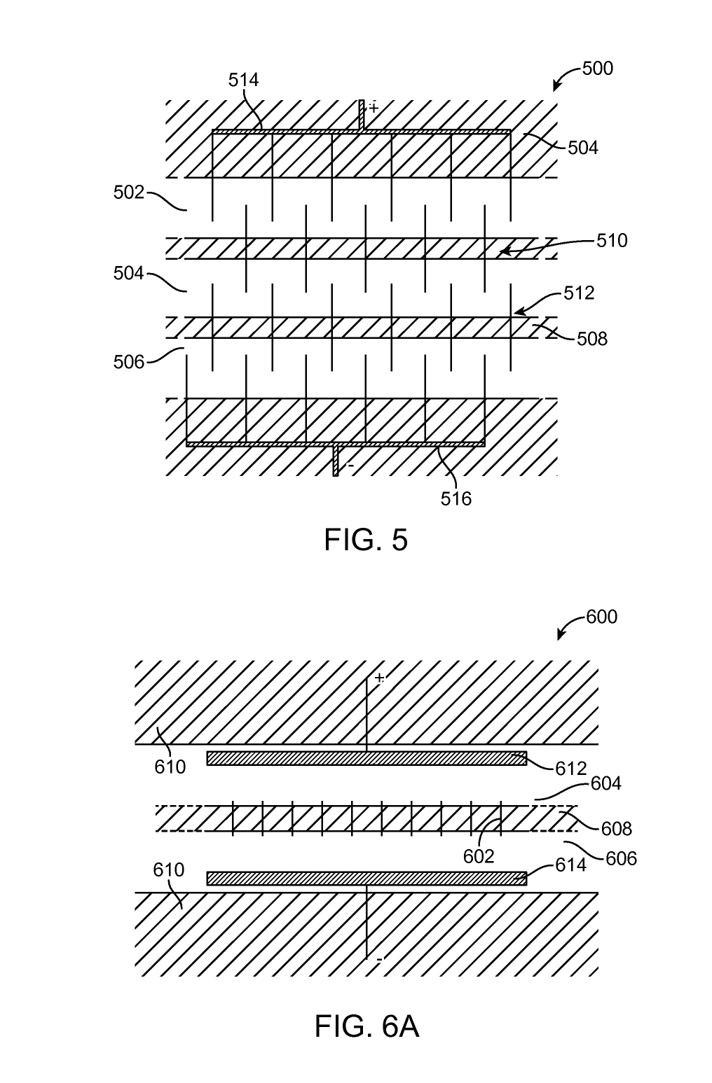

FIG. 5 illustrates a device 500 including a plurality of channels 502, 504, and 506 defined by an insulating material 508. A first row of BPEs 510 spans the channels 502 and 504, while a second row of BPEs 512 spans the channels 504 and 506, with the BPEs in the first row 510 being interdigitated with the BPEs in the second row 512, thereby forming an array of BPEs. The driving electrodes 514, 516 can be comb-like and interdigitated with the plurality of BPEs to generate an electric field that changes sign at each BPE. For example, in the device 500, the driving electrode 514 is interdigitated with the first row of BPEs 510 and the driving electrode 516 is interdigitated with the second row of BPEs 512. Notably, the driving electrodes 514, 516 are in contact with the ionically conductive phase contained in the outermost channels 502, 506, but do not contact the ionically conductive phase contained in the inner channel 504. The BPE array of the device 500 operates based on communication of BPEs with each other. The BPEs in the first row 510 serve as cathodes for the BPEs in the second row 512, while the BPEs in the second row 512 serve as anodes for the BPEs in the first row 510. Although FIG. 5 depicts a single inner channel 504 surrounded by two outer channels 502, 506, it shall be appreciated that this configuration can be extended as desired to include any number of inner channels and rows of BPEs, with each row of BPEs serving as driving electrodes for adjacent rows.

In certain aspects of the present disclosure, the ends of a BPE correspond to electric field maxima in the ionically conductive phase. For example, a single driving electrode can be located along each channel such that the driving electrodes are not in contact with the BPE. The surface area of these driving electrodes can be sufficiently large such that the region of the ionically conductive phase contacting each end of the BPE experiences a local electric field maximum.

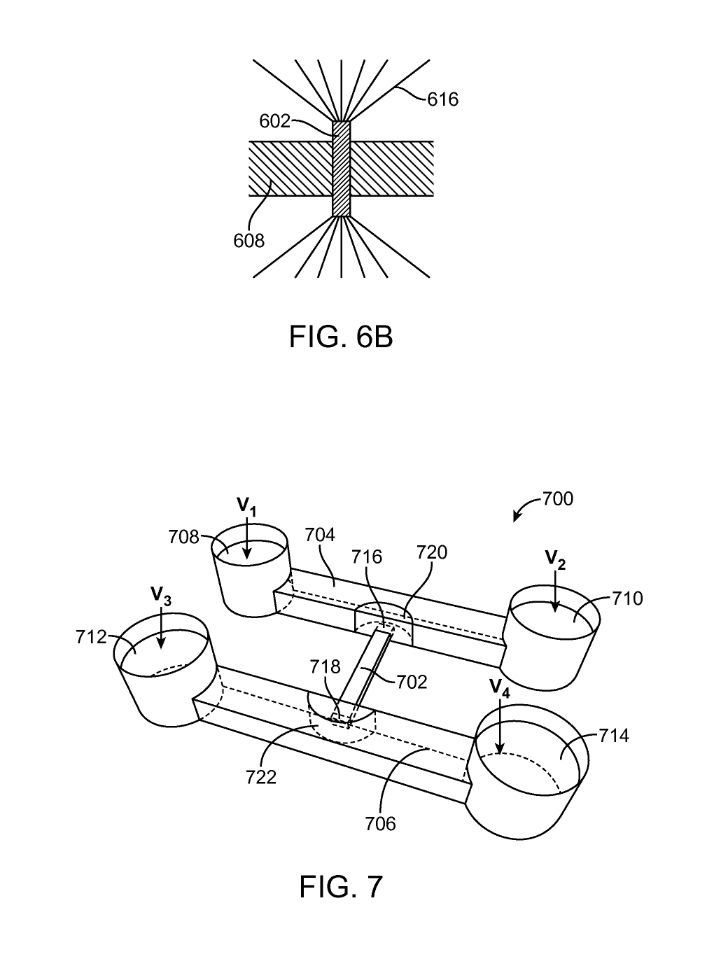

FIG. 6A illustrates a device 600 including a row of BPEs 602 in contact with two channels 604, 606 defined by insulating material(s) 608, 610. Driving electrodes 612, 614 (e.g., conductive plates) extending along the channels 604, 608, can apply the electric field such that local electric field maxima are present at each end of each BPE 602. FIG. 6B illustrates the relative magnitude of the electric field as exemplified by electric field lines 616 in the area immediately surrounding one of the BPEs 602 (from the device 600 of FIG. 6A) spanning the insulating material 608. Regions of relatively higher electric field strength are indicated by more closely spaced electric field lines 616.

### Manipulation of Dielectrophoretic Force by Altering Local Ionic Strength

In some aspects, faradaic processes can lead to an alteration in the distribution and concentration of ions in the ionically conductive phase by processes including charge enrichment and charge depletion. Charge enrichment can comprise electrochemical oxidation or reduction of electrically neutral or zwitterionic species resulting in net charged species. Charge depletion can comprise electrochemical oxidation or reduction of a net charged species to electrically neutral or zwitterionic species. Charge enrichment or depletion, separately or jointly, can lead to local conductivity gradients with associated gradations of electric field strength. An electric field gradient produced in this manner can exert dielectrophoretic force on electrically polarizable discrete phases (e.g., molecules, particles, droplets, cells, etc.).

In certain aspects of the present disclosure, faradaic electrochemistry at the ends of an a BPE perturbs the electric field through the formation of faradaic ion enrichment (FIE) (high conductivity, low field strength) or faradaic ion depletion (FID) (low conductivity, high field strength) zones. Briefly, charge enrichment resulting from faradaic processes at the BPE in either the anodic channel or cathodic channel or both channels can lead to a localized decrease in electric field strength. Depending on the magnitude of ion migration due to convection or electromigration, this local increase in ion concentration (FIE zone) can be centered on the BPE or located at another distance from the BPE. The portion of the ion enrichment zone having the highest ionic conductivity can act as an electric field minimum. The gradient in electric field surrounding this local field minimum can exert dielectrophoretic force on electrically polarizable discrete phases (e.g., molecules, particles, droplets, cells etc.). Specifically, polarizable discrete phases can be accelerated towards (nDEP) or away from (pDEP) this electric field minimum. In some aspects, a plurality of channels and BPEs can be employed to generate an array of electric field minima for dielectrophoretic attraction or repulsion of polarizable discrete phases.

Conversely, charge depletion resulting from faradaic processes at the BPE in either the anodic channel or cathodic channel or both channels can lead to a localized increase in electric field strength. Depending on the magnitude of ion migration due to convection or electromigration, this local decrease in ion concentration (FID zone) can be centered on the BPE or located at another distance from the BPE. The portion of the ion depletion zone having the lowest ionic conductivity can act as an electric field maximum. The gradient in electric field surrounding this local field maximum can exert dielectrophoretic force on electrically polarizable discrete phases (e.g., molecules, particles, droplets, etc.). Specifically, polarizable discrete phases can be accelerated towards (pDEP) or away from (nDEP) this electric field maximum.

As an example, an increase in local ionic strength at the anodic end of a BPE can occur via water oxidation followed by Tris buffer protonation:

4H₂O−2e⁻→2H₃O⁺+O₂ eq. 1

Tris+H₃O⁺→TrisH⁺+H₂O eq. 2

Within the confinement of a microfluidic channel, this increased concentration of cations can remain localized around the anodic end of the BPE. Anions will electromigrate to charge pair with these cations, forming an FIE zone. Any oxidation or reduction reaction adding charge to a solution-phase species can similarly lead to an accumulation of positively and negatively charged ions at either the BPE anode or cathode. Conversely, a decrease in local ionic strength at the cathodic end of a BPE can occur via the following set of reactions:

2H₂O+2e⁻→2OH⁻+H₂ eq. 3

TrisH⁺+OH⁻→Tris+H₂O eq. 4

The net result of this series of reactions is the neutralization of the buffer cation, TrisH+ to neutral Tris. In this case, the co-anion (CL) migrates away from the site of neutralization, thus leading to localized FID at the BPE cathode. Likewise, the neutralization of any charged species can lead to an FID zone. Significantly, the position of the FIE and FID zones can be controlled using convection, as described further herein.

In some aspects, the ionically conductive phase and actuating electrode can be contained within a fluidic channel. The channel can have openings (inlets and outlets) for the actuation of convective flow, introduction of objects, and application of the electric field. A plurality of actuating electrodes arranged in an array format can be used to generate multiple charge enrichment and charge depletion zones through faradaic processes.

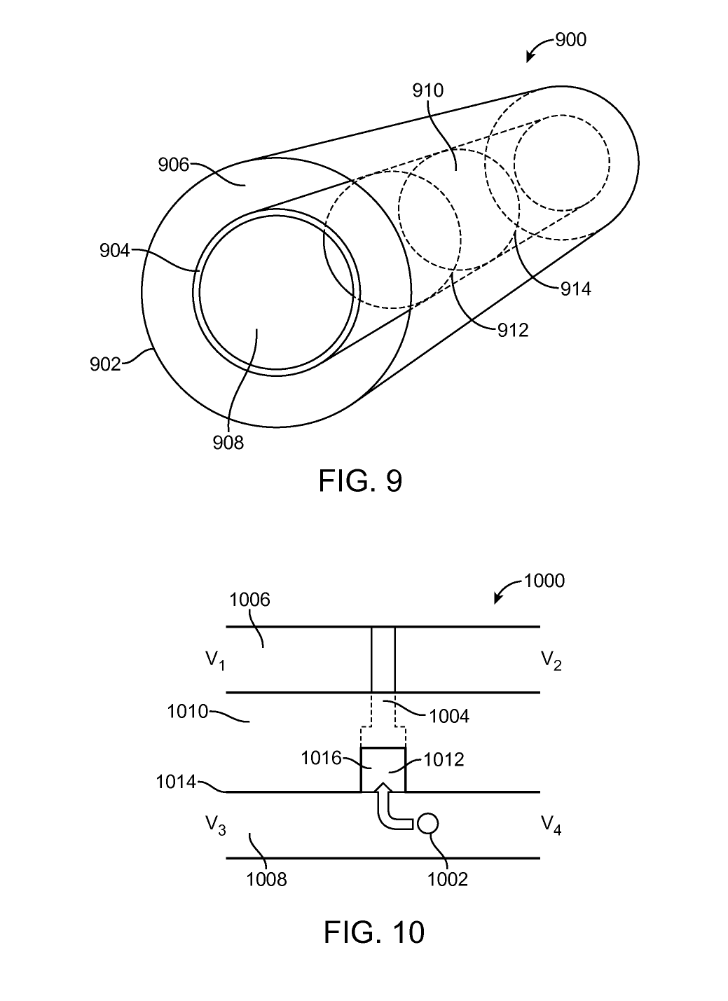

In other aspects, the actuating electrode is a BPE contacting two channels. In this embodiment, a voltage bias can be applied across the two channels such that coupled oxidation and reduction reactions occur at separate ends of the BPE in contact with the ionically conductive phase in each channel. Specifically, in one channel (the cathodic channel), the electrical potential of the ionically conductive phase in contact with the BPE can be higher (more positive) than the electrical potential of the BPE, leading to electron transfer from the BPE to chemical species in the ionically conductive phase (electrochemical reduction). In the other channel (the anodic channel), the electrical potential of the ionically conductive phase in contact with the BPE can be lower (more negative) than the electrical potential of the BPE, leading to electron transfer from chemical species in the ionically conductive phase to the BPE (electrochemical oxidation). This embodiment can be realized using any of the devices described in FIG. 3A, FIG. 4B, FIG. 4C, FIG. 5, FIG. 6A, FIG. 7, or FIG. 9.

FIG. 7 illustrates a dual-channel device 700 similar to the device 300 of FIG. 3A. The device 700 includes a BPE 702 having fluidically isolated ends, with one end being in electrical communication with an ionically conductive phase in a first fluidic channel 704 and the opposing end being in electrical communication with an ionically conductive phase in a second fluidic channel 706. The first and second fluidic channels 704, 706 are connected to reservoirs 708, 710, and 712, 714, respectively. Voltages V₁, V₂, V₃, and V₄, are applied at reservoirs 708, 710, 712, and 714, respectively. In some aspects, a DC voltage bias is applied across the first and second fluidic channels 704, 706 such that the first fluidic channel 704 is a cathodic channel and the second fluidic channel 706 is an anodic channel. Accordingly, the solution potential in contact with the BPE 702 in the cathodic channel is higher than the BPE potential (U_{BPE}) and the solution potential in contact with the BPE 702 in the anodic channel is lower than U_{BPE} (see, e.g., FIG. 3B). Accordingly, the BPE 702 can comprise a BPE cathode 716 at the BPE end situated in the first fluidic channel 704 and a BPE anode 718 at the BPE end situated in the second fluidic channel 706. When a sufficiently large voltage bias is applied across the cathodic and anodic channels, FID zone 720 and FIE zone 722 are formed at the BPE cathode 716 and BPE anode 718, respectively. A key advantage of this device configuration is that the applied DC voltage required to drive faradaic processes can be significantly lower than in the single channel design. This improvement is owed to the removal of an ionic current path (fluidic junction) between the anodic and cathodic driving electrodes.

Some aspects of the present disclosure utilize FIE and FID zones for dielectrophoretic manipulation of objects. These conductivity gradients act as extensions to the BPE, thus impacting a larger volume than the electric field gradients surrounding a typical planar electrode. The conductivity gradients described herein can extend at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 150 μm, at least about 200 μm, at least about 250 μm, at least about 300 μm, at least about 350 μm, at least about 400 μm, at least about 450 μm, at least about 500 μm, at least about 550 μm, at least about 600 μm, at least about 650 μm, at least about 700 μm, at least about 750 μm, at least about 800 μm, at least about 850 μm, at least about 900 μm, at least about 950 μm, at least about 1 mm, or at least about 5 mm from the BPE.

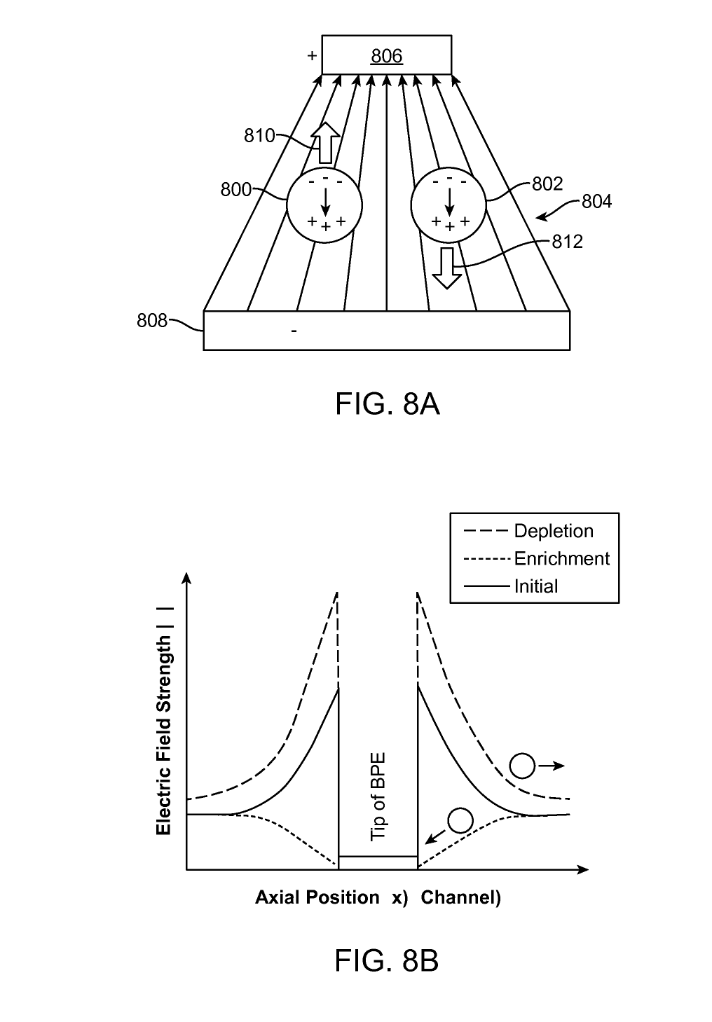

FIG. 8A illustrates polarizable objects 800, 802 (depicted herein as particles) in an electric field 804 generated by a cathodic driving electrode 806 and anodic driving electrode 808. A polarizable object subjected to an electric field will develop an induced dipole moment p. The magnitude of the dipole depends upon the volume of the object, its degree of polarizability, and the strength of the surrounding electric field (E). In the presence of an electric field gradient, the object will be attracted to regions of higher |E| if the complex permittivity of the particle (εₚ*) is greater than the complex permittivity of the surrounding medium (εₘ*). This condition is called positive dielectrophoresis (pDEP) (arrow 810). Conversely, negative dielectrophoresis (nDEP) (arrow 812) will occur if εₚ* is less than εₘ*. The magnitude of dielectrophoretic force (F_{DEP}) exerted on a spherical particle is given by the following equation.

F_{DEP}=2πr³εₘ*Re[K(ω)]V|E²| eq. 5

Here, r is the particle radius and Re[K (cu)] is the real part of the Clausius-Mossotti factor (K), which is a function of electric field frequency (ω).

$$\begin{matrix}K = ( {ɛ}_{p}^{*} - {ɛ}_{m}^{*} ) / ( {ɛ}_{p}^{*} + 2 {ɛ}_{m}^{*} ) & eq . 6 \\ {ɛ}^{*} = ɛ + ( \frac{iσ}{ω} ) & eq . 7\end{matrix}$$

Equations 5-7 highlight the dramatic impact that a local change in solution conductivity (G) can have on F_{DEP}. Specifically, the formation of an FID zone leads to an ohmic increase in the local magnitude of E, and simultaneously, causes εₘ* to decrease (making K more positive). Likewise, FIE can have the opposite effect on E and εₘ*. This synergistic effect is important because, as a particle is attracted (for instance, by pDEP into a high |E| region), the magnitude of K can increase, leading to amplified attraction.

FIG. 8B illustrates the anticipated impact of FIE and FID on the axial component of the electric field adjacent to either end of a BPE in a microfluidic device (e.g., the device 700 of FIG. 7). This simplified depiction assumes that the driving voltage applied to the device is symmetrical (e.g., in the device 700 V₁=V₂ and V₃=V₄). In certain aspects, the electric field surrounding a BPE that is active (i_{BPE}≠0) is zero directly above the BPE and enhanced at the BPE edges (FIG. 8B, solid line). The formation of an FIE zone leads to an ohmic decrease in the local magnitude of E, with the greatest impact nearest the BPE. At the BPE, the electric field remains zero. A cell can be trapped by nDEP at the resulting electric field minimum, at which the cell has a reduced risk of electric field-induced damage. Conversely, FID leads to an increase in the local magnitude of E, which can lead to enhanced and extended nDEP repulsion of a cell from the BPE.

In various aspects, an electric field gradient formed by FIE and FID can extend up to several hundred microns from the BPE. Tables 1 and 2 show the estimated nDEP force experienced by 10- and 20-μm diameter cells at the field maxima of electric field gradients attainable by FIE and FID:

TABLE 1
Effect of electric field gradient length on F_{DEP}
(30 kV/m-0 kV/m gradient).

| | F_{DEP,max} (pN) | |
|---|---|---|
| Length of gradient (μm) | 10-μm diameter cell | 20-μm diameter cell |
| 300 | 1.7 | 13.3 |
| 200 | 2.6 | 19.9 |
| 100 | 4.9 | 39.9 |
| 50 | 10.2 | 80.1 |

TABLE 2
Effect of electric field gradient length on F_{DEP}
(50 kV/m-0 kV/m gradient).

| | F_{DEP,max} (pN) | |
|---|---|---|
| Length of gradient (μm) | 10-μm diameter cell | 20-μm diameter cell |
| 300 | 4.8 | 37.0 |
| 200 | 7.2 | 55.4 |
| 100 | 13.6 | 111 |
| 50 | 28.2 | 222 |

As a point of reference, the drag force experienced by these cells moving through solution at 20 μm/s is 1.9 pN and 3.8 pN, respectively, and the drag force when moving through solution at 40 μm/s is 3.8 pN and 7.5 pN, respectively. Although stronger fields may be used in certain applications, in some aspects, the maximum field strengths shown here are limited by the threshold applied transmembrane potential for electroporation (e.g., approximately 0.5 V). The exact threshold at which electroporation occurs is determined by the solution conditions (e.g., conductivity), cell membrane characteristics, and pattern of the applied field. The applied transmembrane potential at any point along the cell surface can be calculated using the Schwan equation, which assumes a spherical cell:

Uₜᵣₐₙₛ=−1.5rE cos φ eq. 8

where Uₜᵣₐₙₛ is the applied transmembrane potential and cp is the angle between the local electric field and a line extending from the cell center to the location of interest on the cell membrane. Given a threshold of Uₜᵣₐₙₛ=0.5 V, E is maintained below 33 kV/m for a 10-μm diameter cell.

Various aspects of the present disclosure can be employed individually or in various combinations for dielectrophoretic manipulation of objects such as discrete polarizable phases. In one aspect, faradaic reactions at a BPE generate an ion enrichment zone, and the decrease in electric field strength in the ionically conductive phase surrounding the BPE attracts polarizable objects towards the BPE via negative dielectrophoresis. Concomitantly, the electric field is applied in such a way that the electric field strength is zero in the segment of the ionically conductive phase contacting the BPE. In this way, ion enrichment and a change in electric field sign at the BPE can be used in concert to attract and trap polarizable objects at the BPE.

In other aspects, a polarizable object can be located on the BPE surface (or between BPEs) prior to application of the electric field. The electric field can then be applied in such a manner that the electric field strength at the BPE is zero while an ion depletion zone forms in the ionically conductive phase surrounding the BPE. In this case, the electric field surrounding the BPE is enhanced (increased in magnitude) with a region of zero electric field strength centered on the BPE. A polarizable object can be trapped in this zero-field region (at the BPE) by nDEP, and the surrounding enhanced field will reinforce the trapping strength via exclusion of the polarizable object based on repulsive nDEP force.

In certain aspects, an ion enrichment zone and zero electric field strength at the BPE can be used to attract a polarizable object by nDEP. Subsequently, the electric field conditions can be altered to replace the ion enrichment zone with an ion depletion zone. This approach can result in enhanced trapping of the polarizable object and repulsion of (or prevention of trapping) further polarizable objects.

### Application of BPE Technologies for Dielectrophoretic Manipulation of Objects

The present disclosure provides methods, systems, and devices that can be applied in a variety of ways to manipulate objects such as particles, discrete polarizable phases, cells, and the like. In some aspects, the present disclosure enables manipulation of the position of an object or a plurality of objects. For example, objects can be captured or trapped, either individually or as a group, at electric field minima (via nDEP) or electric field maxima (via pDEP). Cessation of capture conditions (e.g., by turning off the applied field or by disrupting faradaic processes) can lead to controlled release of the trapped object. Furthermore, electric field minima and maxima produced via the methods of the present disclosure can be employed to adjust the position or velocity of objects in a flowing ionically conductive phase. Applications include but are not limited to: positioning an object within a flow lamina; hydrodynamic focusing based on repeated application of dielectrophoretic force; or mixing and sorting based on switching dielectrophoretic force on and off or based on differential attraction or repulsion of multiple discrete polarizable phases due to differences in their dielectrophoretic properties.

In certain aspects, the methods, systems, and devices of the present disclosure can be used to locally enrich the concentration of objects via dielectrophoretic force. In this embodiment, a BPE (or multiple BPEs) can be used to generate a region of low or zero electric field strength in a segment of an ionically conductive phase (e.g., within a fluidic channel or well). Objects flowing through the channel can be decelerated or trapped at the low/zero field segment via nDEP, and as a result, their concentration can become enriched in this segment.

FIG. 9 illustrates a cylindrical device 900 for concentration enrichment. Two concentric cylinders (outer cylinder 902 and inner cylinder 904) comprised of insulating material define two channels (outer channel 906 and inner channel 908) through which an ionically conductive phase carrying objects can be flowed. An electric field can be applied at the inlet and outlet of the channels 906, 908, and the electric field can be selected to have properties appropriate for nDEP of the objects to be trapped. In some aspects, the inner cylinder 904 includes a conductive segment 910 (bounded by circles 912, 914) that serves as a BPE. The segment 910 can comprise a conductive material, a pair or series of conductive rings or cylinders, or an array of BPEs in contact with the ionically conductive phase in both channels 906 and 908. For example, the device 900 can include a pair of ring-shaped BPEs (e.g., positioned at circles 912, 914). This conductive segment 910 can generate a region of low or zero electric field strength at which objects carried by the flowing ionically conductive phase can be trapped and accumulated.

The design of the device 900 can be varied as desired. In some aspects, the length of the cylinders 902, 904 can be at least about 100 μm, at least about 200 μm, at least about 250 μm, at least about 300 μm, at least about 400 μm, at least about 500 μm, at least about 600 μm, at least about 700 μm, at least about 800 μm, at least about 900 nm, at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, at least about 15 mm, at least about 20 mm, at least about 30 mm, at least about 40 mm, or at least about 50 mm. The inner diameter of the inner cylinder 904 can be at least about 5 μm, at least about 10 μm, at least about 50 μm, at least about 100 μm, at least about 500 μm, at least about 1 mm, or at least about 5 mm. The thickness of the inner cylinder wall can be at least about 1 μm, at least about 5 μm, at least about 10 μm, at least about 50 μm, at least about 100 μm, at least about 500 μm, or at least about 1 mm. The inner diameter of the outer cylinder 902 can be sufficiently large so as to encompass the outer diameter of the inner cylinder 904. For example, the inner diameter of the outer cylinder 902 can be at least about 10 μm, at least about 50 μm, at least about 100 μm, at least about 500 μm, at least about 1 mm, at least about 5 mm, at least about 10 mm, or at least about 50 mm. The length of the conductive segment 910 can be at least about 1 μm, at least about 5 μm, at least about 10 μm, at least about 50 μm, at least about 100 μm, at least about 500 μm, at least about 1 mm, or at least about 10 mm.

In certain aspects, the systems and devices of the present disclosure can comprise structures shaped to facilitate the manipulation of objects. For instance, entrapment structures such as chambers, compartments, wells, notches, cavities, or suitable combinations thereof can be used to physically constrain the position of an object. Such entrapment structures can limit the movement of the object along certain directions so as to facilitate trapping of the object at a desired location. The entrapment structure can be aligned with a corresponding BPE so that the dielectrophoretic forces applied by the BPE direct the object into the entrapment structure. In some aspects, the dimensions of the entrapment structure can be designed in order to limit the number of captured objects, e.g., a chamber sized to accommodate a single cell. Once an object or objects have been trapped within a entrapment structure, additional manipulations or analyses can be performed, as described further herein.

FIG. 10 illustrates a device 1000 for facilitating trapping of an object 1002. The device 1000 includes a BPE 1004 having one end situated in an auxiliary channel 1006 and an opposing end situated in a dielectrophoresis channel 1008. The auxiliary channel 1006 and dielectrophoresis channel 1008 are defined and fluidically isolated from each other by an insulating material 1010 (e.g., PDMS). Each channel 1006, 1008 contains an ionically conductive phase. In some aspects, the ionically conductive phase within the auxiliary channel 1006 is the same as the ionically conductive phase within the dielectrophoresis channel, while in other aspects, the ionically conductive phases are different. Driving voltages (V₁, V₂, V₃, and V₄) can be applied to each ionically conductive phase via reservoirs at the ends of each channel 1006, 1008, similar to the dual-channel devices described herein.

In some aspects, the dielectrophoresis channel 1008 includes a chamber 1012 formed in the channel wall 1014. The chamber 1012 is aligned to a tip 1016 of the BPE 1004 such that the tip 1016 extends into the chamber 1012 and is exposed to the ionically conductive phase within the dielectrophoresis channel 1008. The object 1002 can be introduced into the dielectrophoresis channel 1008 (e.g., via a reservoir or inlet) and brought within proximity of the BPE 1004 by convective flow. Upon application of an appropriate electrical field across the dielectrophoresis channel 1008 and the auxiliary channel 1006, nDEP forces generated by the BPE 1004 as described herein attract the object 1002 introduced in the dielectrophoresis channel 1008 towards the segment of the ionically conductive phase near the BPE 1004 and into the chamber 1012. Maintenance of the electrical field results in trapping of the object 1002 within the chamber 1012, while cessation of the electrical field permits release of the object 1002.

In other aspects of the present disclosure, directing structures can be used to direct the movement of an object. Examples of such directing structures include channels, passages, outlets, inlets, branching structures, and the like. The directing structures can be shaped and aligned with the BPE such that the application of dielectrophoretic force by the BPE influences the movement of the object relative to the directing structures. For example, a directing structure can comprise a branching point linked to a plurality of passages and dielectrophoretic forces can be used to control the movement of the object into a selected one of the plurality of passages.

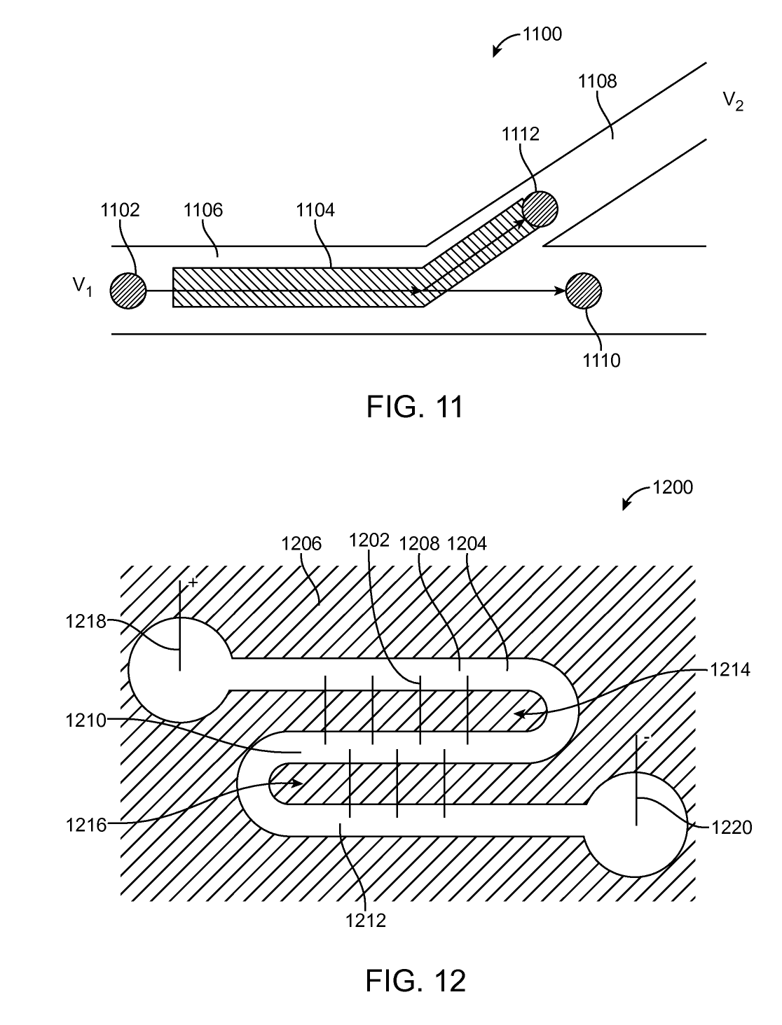

FIG. 11 illustrates a device 1100 for directing the movement of an object 1102. The device 1100 includes a BPE 1104 having a portion situated within a first channel 1106 and a portion situated within a second channel 1108. The second channel 1108 is fluidly connected to the first channel 1106 and serves as an outlet for fluid flow from the first channel 1106. The BPE 1104 is shaped so as to conform to the geometry of the branching point between the first and second channels 1106, 1108. The first and second channels 1106, 1108 contain an ionically conductive phase, the electric field of which can be controlled by voltages V₁, V₂ applied at opposing ends of the first and second channels 1106, 1108, respectively. In the absence of applied voltage, when the object 1102 is introduced into the first channel 1106, the object 1102 will tend to pass through the first channel 1106 (e.g., to position 1110) without entering the second channel 1108. The movement of the object 1102 can be biased to remain within the first channel 1106, e.g., by convective flow or based on the angle between the first and second channels 1106, 1008. When an appropriate voltage is applied across the ionically conductive phase, nDEP forces generated by the BPE 1104 as described herein divert the object 1102 from the first channel 1106 into the second channel 1108 (e.g., to position 1112). Although the device 1100 is depicted as including only two channels, it shall be appreciated that the present disclosure can be extended to any number of connected channels, and the BPE arrangement can be varied as desired in order to enable selective direction of objects into any one of the channels.

In various aspects, the trapping phenomena described herein are exploited for parallel capture of multiple objects. Significantly, BPEs can function in parallel in which case a single pair of driving electrodes can supply the electrical potential drop across the solution to drive redox processes at each and every BPE. Accordingly, some aspects of the present disclosure provide manipulation of multiple objects in an array-based format. For example, multiple BPEs can be arranged in an array configuration (e.g., device 200 of FIG. 2, device 500 of FIG. 5, device 600 of FIG. 6A) so as to permit trapping of multiple objects at multiple locations over a 2D surface area or a 3D volume. In various aspects, such BPE arrays can be used in conjunction with a corresponding array of entrapment structures (e.g., chambers, wells, etc.) and/or directing structures to facilitate object trapping. The array formats described herein can be used to achieve parallel processing of cells and other objects. Advantageously, the use of array-format systems and devices as described herein can enable individual manipulation of a large number of objects simultaneously, as well as facile separation and entrapment of objects in a desired format (e.g., a well format). A key advantage of array-based trapping is that objects can be ordered and isolated for parallel processing. For example, individual cells can be trapped, lysed, and then loaded into a separate channel or chamber for PCR analysis. Similarly, cells can be swelled and porated for parallel gene transfection. Additional examples of analysis and processing procedures that can be performed on objects are described further herein.

FIG. 12 illustrates a device 1200 in which an array of BPEs 1202 are in contact with a single serpentine channel 1204 defined by an insulating material 1206. The serpentine channel 1204 includes a first portion 1208, second portion 1210, and a third portion 1212. A first row of BPEs 1214 is in contact with the first and second portions 1208, 1210 and a second row 1216 of BPEs is in contact with the second and third portions 1210, 1212. Driving electrodes 1218, 1220 can be located at the ends of the serpentine channel 1204. In some aspects, the complex electric field profile leads to an array of dielectrophoretic forces that can be used to manipulate the positions of multiple objects simultaneously.

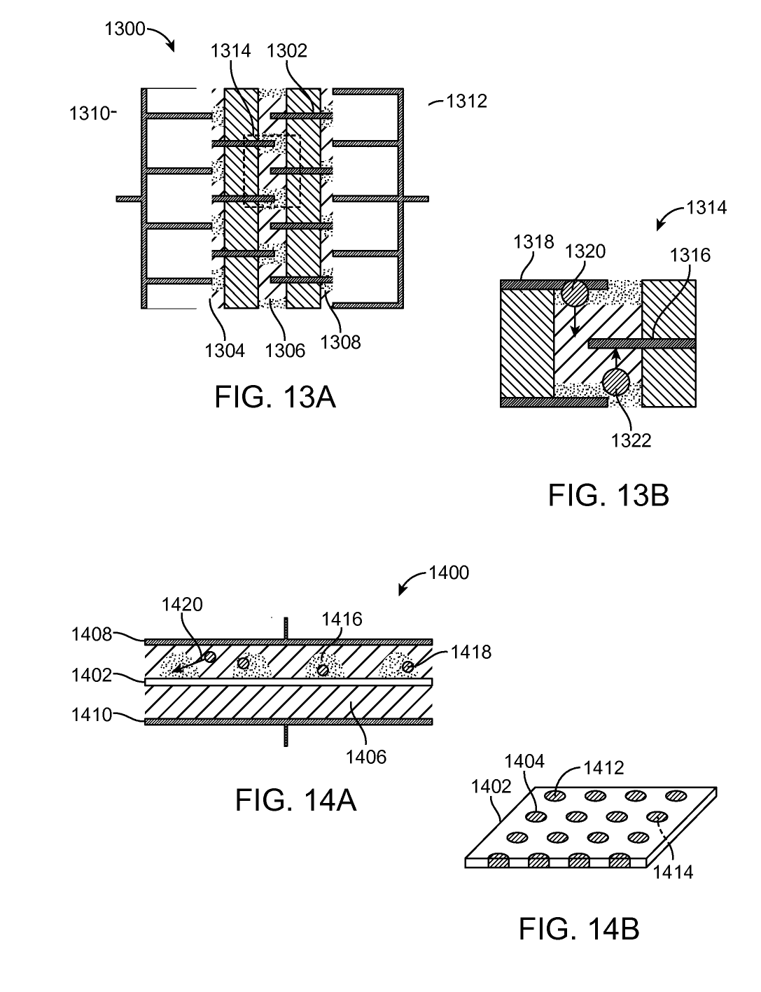

FIGS. 13A and 13B illustrate a multi-channel device 1300 for array-based nDEP trapping of objects. Similar to the device 500 of FIG. 5, the device 1300 includes a plurality of BPEs 1302 arranged in interdigitating rows and three fluidic channels 1304, 1306, 1308. Driving electrodes 1310, 1312 located at opposite ends of the device 1300 are interdigitated with the BPEs 1302. This configuration will lead to a sinusoidal potential profile along the fluidic channels 1304, 1306, 1308 with derivative (E) equal to 0 at the tip of each BPE 1302 and at each extension of the driving electrodes 1310, 1312. FIG. 13B is a close view of the region 1314 of FIG. 13A. Faradaic reactions can lead to ion enrichment around the anodes 1316 and ion depletion around the cathodes 1318. If the field frequency is appropriate for nDEP, objects will be repelled from the cathodes (via ion depletion zones, e.g., object 1320) and attracted to the anodes (via ion enrichment zones, e.g., object 1322).

FIGS. 14A and 14B illustrate a device 1400 for array-based pDEP trapping of objects. The device comprises a membrane 1402 containing an array of BPEs 1404. The BPEs 1404 are integrally formed with the membrane 1402 such that the length of each BPE 1404 extends through the thickness of the membrane 1402. The membrane 1402 is immersed in an ionically conductive phase 1406 situated between two parallel plate electrodes (cathode 1408 and anode 1410). The upper surface of each BPE 1404 is the cathodic end 1412 and the lower surface is the anodic end 1414. In this scheme, an ion depletion zone 1416 is formed at the cathodic end of each BPE. Objects 1418 will be attracted towards the center of the depletion zones 1416 by pDEP forces (e.g., arrow 1420). Importantly, an anionic buffer (e.g., carbonate buffer) can be employed to generate similar depletion zones in the anodic compartment (e.g., by neutralization of carbonate ions).

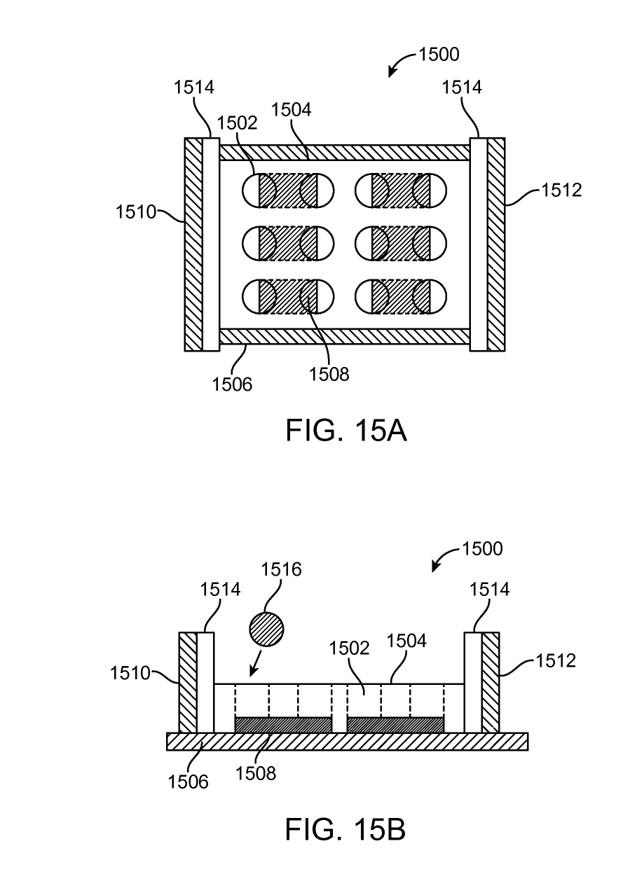

FIGS. 15A and 15B illustrate a device 1500 for array-based trapping of objects in a well format. The device 1500 includes an array of wells 1502 formed in an insulating material 1504 (e.g., a PDMS monolith). Each of the wells 1502 are fluidically isolated from each other. The device 1500 can be partially or wholly immersed in an ionically conductive phase such that the wells 1502 are filled with the ionically conductive phase. The insulating material 1504 comprising the array of wells 1502 is positioned over a substrate 1506. An array of planar BPEs 1508 is formed on the substrate 1506. The array of BPEs 1508 is aligned with the array of wells 1502 such that each BPE 1508 spans a different pair of adjacent wells, with one end positioned at the bottom surface of each well 1502 and exposed to the ionically conductive phase in the well 1502. A pair of planar driving electrodes 1510, 1512 are positioned at either end of the insulating material 1504, with a protective porous membrane 1514 interspersed between each driving electrode 1510, 1512 and the insulating material 1504. Following the introduction of objects 1516, a suitable electric field can be applied by the driving electrodes 1510, 1512 so as to attract and trap the objects 1516 in the wells 1502. The wells 1502 and trapping conditions can be designed such that a single object 1516 is trapped in each well 1502.

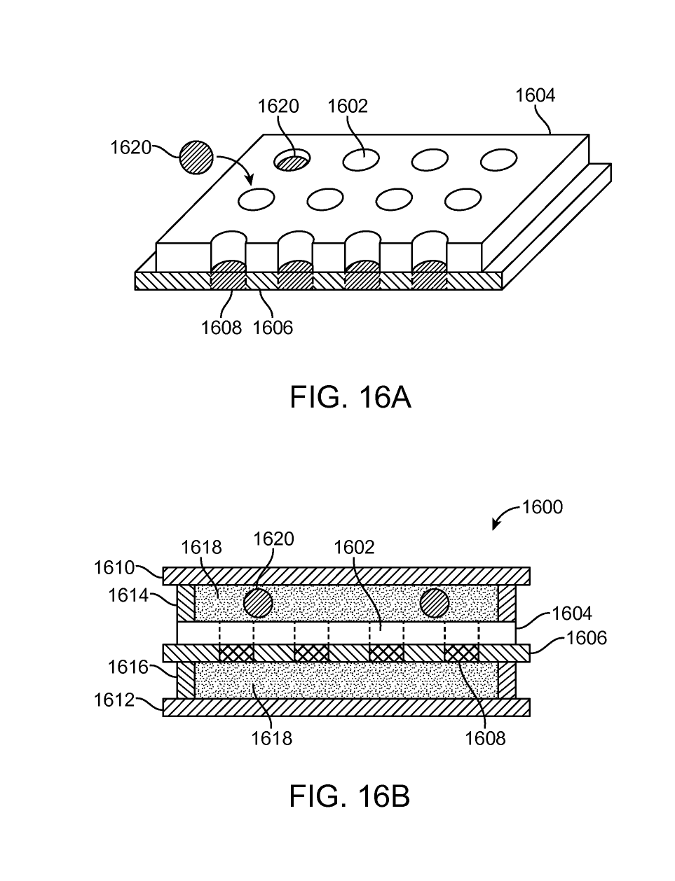

FIGS. 16A and 16B illustrate a device 1600 for array-based trapping of object in a well format. Similar to the device 1500 of FIGS. 15A and 15B, the device 1600 includes an array of wells 1602 formed in an insulating material 1604 (e.g., a PDMS monolith), with the wells 1602 being fluidically isolated from each other. The insulating material 1604 is positioned over a substrate 1606. The substrate 1606 comprises an array of integrally formed BPEs 1608, with the length of each BPE 1608 extending through the thickness of the substrate 1606. The array of BPEs 1608 is aligned with the array of wells 1602 such that the upper end of each BPE 1608 is exposed through the bottom surface of a different corresponding well 1602. The insulating material 1604 and substrate 1606 are positioned between two planar driving electrodes 1610, 1612 with gaps between the upper driving electrode 1610 and insulating material 1604, and the substrate 1606 and the lower driving electrode 1612 that are respectively fluidically sealed with gaskets 1614, 1616. The intervening spaces between the driving electrodes 1610, 1612, the insulating material 1604, and the substrate 1606 is filled with ionically conductive phase 1618 so as to fill the wells 1602. Objects 1620 can be introduced into the upper ionically conductive phase in contact with the wells 1602. An electric field can be applied by the driving electrodes 1610, 1612 in order to attract and trap the objects 1620 into the wells 1602. The geometry of the wells 1602 and the trapping conditions can be selected such that a single object 1620 is trapped in each well 1602.

### Methods, Systems, and Devices for Manipulation and Analysis of Samples

The methods, systems, and devices of the present disclosure can be applied to the manipulation and analysis of a wide variety of samples, such as biological samples (e.g., blood samples, plasma samples, serum samples, solutions that contain cell lysates or secretions or bacterial lysates or secretions, and other biological samples containing proteins, bacteria, viral particles and/or biological cells (eukaryotic, prokaryotic, or particles thereof), cellular fractions and lysates). In some aspects, a sample is attracted towards a BPE using the methods described herein. In other aspects, a sample is repelled away from a BPE using the methods described herein. The present disclosure can be applied to trap or capture samples near a single BPE or multiple BPEs, e.g., in an array-based format.

In some aspects, the methods, systems, and devices of the present disclosure can be used to process and analyze trapped samples. The systems and devices described herein can be used in combination with systems and devices configured to process samples in a variety of ways, including but not limited to treatment (e.g., lysis, fusion, amplification, mixing with an analysis reagent), displacement, collection, removal, separation (e.g., discretization or isolation, such as in droplets), analysis (e.g., detection), or suitable combinations thereof. The systems and devices of the present disclosure can be coupled to upstream (before dielectrophoretic manipulation) or downstream (after dielectrophoretic manipulation) devices or systems for the generation, pre-treatment, post-treatment, or analysis of the sample or the ionically conductive phase (e.g., a segment of the ionically conductive phase containing the sample). For example, following trapping of a sample as described herein, a removal device can be used to displace the sample from the trapping location, e.g., for further processing or to allow entrapment of another sample. As another example, a collection device can be used to collect the trapped sample into a suitable container (e.g., well, plate, tube, chamber, etc.), e.g., for storage or analysis. In various aspects, a trapped sample can be analyzed, in situ or following displacement or collection, e.g., using a detection device configured to detect the sample or a component thereof.

In certain aspects of the present disclosure, sample analysis is performed by combining the sample with an analysis reagent, such as an amplification reagent or a detection reagent as described herein. In some aspects, the analysis reagent is provided with the sample (e.g., provided in the ionically conductive phase containing the sample). In other aspects, the analysis reagent is introduced separately from the sample (e.g., prior to or after introduction of the sample) and subsequently mixed with the sample. For example, the sample can be trapped within an entrapment structure and the analysis reagent can be subsequently introduced into the entrapment structure. Alternatively, the analysis reagent can be trapped or immobilized within an entrapment structure and the sample can be subsequently introduced into the entrapment structure. As another example, the sample and analysis reagent can each be trapped within respective entrapment structures and then mixed together (e.g., via convection, electrokinetic transport, displacement, etc.).

For example, in some aspects, the sample can comprise a polynucleotide sample that is amplified, e.g., via mixing with an amplification reagent. The methods, systems, and devices of the present disclosure can be used to amplify a polynucleotide sample, such as with polymerase chain reaction (PCR), reverse transcriptase PCR (RT-PCR), ligase chain reaction (LCR), loop mediated amplification (LAMP), reverse transcription loop mediated amplification (RT-LAMP), helicase dependent amplification (HDA), reverse transcription helicase dependent amplification (RT-HDA), recombinase polymerase amplification (RPA), reverse transcription recombinase polymerase amplification (RT-RPA), catalytic hairpin assembly reactions (CHA), hybridization chain reaction (HCR), entropy-driven catalysis, strand displacement amplification (SDA), and/or reverse transcription strand displacement amplification (RT-SDA). In certain aspects, the apparatus, devices, methods and systems of the present disclosure can be used for nucleic acid sequence based amplification (NASBA), transcription mediated amplification (TMA), self-sustained sequence replication (3SR), and single primer isothermal amplification (SPIA). Other techniques that can be used include, e.g., signal mediated amplification of RNA technology (SMART), rolling circle amplification (RCA), hyper branched rolling circle amplification (HRCA), exponential amplification reaction (EXPAR), smart amplification (SmartAmp), isothermal and chimeric primer-initiated amplification of nucleic acids (ICANS), and multiple displacement amplification (MDA). The amplification reagent can be selected from a polymerase chain reaction (PCR) reagent, rolling circle amplification (RCA) reagent, nucleic acid sequence based amplification (NASBA) reagent, loop-mediated amplification (LAMP) reagent or a combination thereof. In some aspects, the amplification reagent is a PCR reagent. In certain aspects, the PCR reagent is selected from a thermostable DNA polymerase, a nucleotide, a primer, probe or a combination thereof.

In further aspects, a sample can be mixed with a detectable agent, wherein the detectable agent is capable of labeling the sample. In some aspects, the sample is labeled with a detectable agent. In further aspects, the detectable agent is capable of binding a nucleic acid sample. Various detectable agents can be used according to the present disclosure. In various aspects, the detectable agent is fluorescent. In further aspects, the detectable agent is luminescent. The detectable agent used can depend on the type of amplification method that is employed. In one aspect, the signal generation can come from a nonsequence specific fluorophore such as EvaGreen or SYBRgreen, where the fluorophore is quenched when in solution but can intercalate into double-stranded DNA where it exhibits much brighter fluorescence. Thus the large amount of double stranded DNA generated during PCR results in a significant increase in fluorescence. In another aspect sequence specific fluorescent probes are used. In one aspect this consists of a molecular beacon such as a hairpin structure, whose fluorescence is highly quenched in its closed conformation and whose intensity is increased once it hybridizes to amplified target DNA. In another aspect it consists of a Taqman probe, which hybridizes to the target DNA, and undergoes cleavage of a fluorescent reporter from the probe DNA during the next amplification step.

In some aspects, sample analysis is performed by using a detection device to detect the presence or absence of an analyte in the sample, e.g., directly or via a coupled detection reagent. For example, the detection device can be configured to perform imaging, such as optical imaging. The optical imaging can be performed by confocal microscopy, spinning disk microscopy, multi-photon microscopy, planar illumination microscopy, Bessel beam microscopy, differential interference contrast microscopy, phase contrast microscopy, epifluorescent microscopy, bright field imaging, dark field imaging, oblique illumination, or a combination thereof.

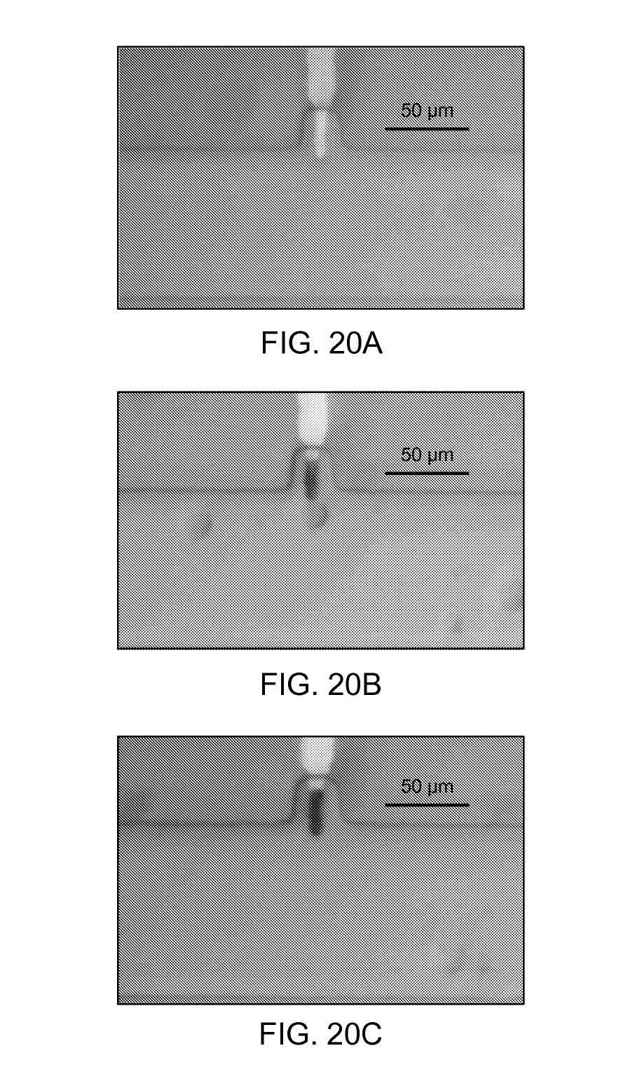

In some aspects, a sample can comprise biological compartments. A biological compartment is typically defined by the presence of an enveloping (enclosing) lipid membrane. Some examples include eukaryotic and prokaryotic cells, vesicles, and organelles. The methods, systems, and devices of the present disclosure can be used to capture or trap biological compartments, either individually (e.g., FIG. 20A through 20C) or as a group (e.g., FIGS. 21A through 21C), at electric field minima (via nDEP) or electric field maxima (via pDEP). Cessation of capture conditions (e.g., by turning off the applied field or by disrupting faradaic processes) can lead to controlled release of the trapped biological compartment.

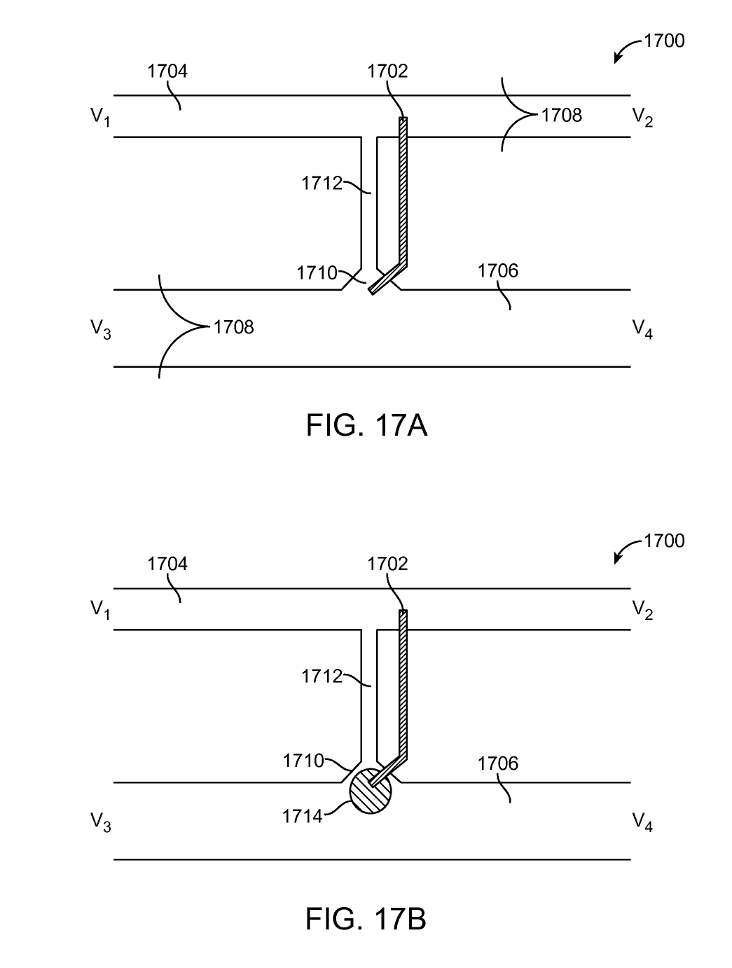

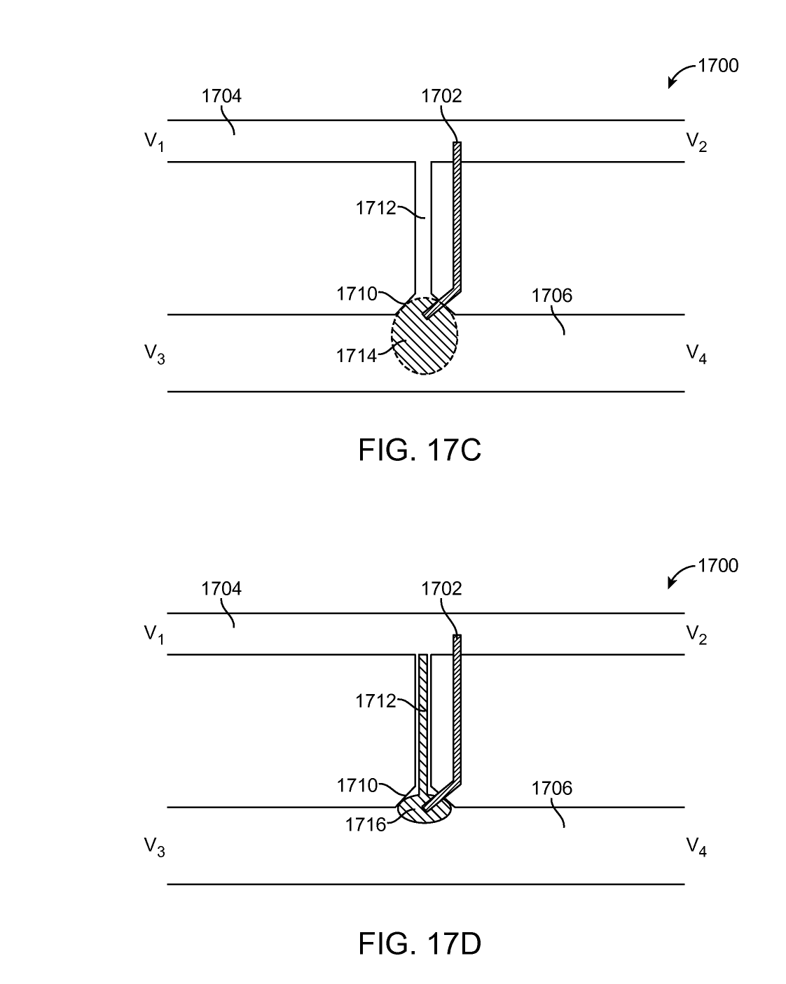

In certain aspects (e.g., FIGS. 17A through 17D), biological compartments can be trapped as described herein and then subsequently subjected to ion depletion of the surrounding medium. Ion depletion surrounding the biological compartment can lead to increased osmotic pressure leading to swelling (fluid uptake) of the biological compartment. Swelling separately or in combination with locally increased electric field strength can cause pores to form in the lipid membrane of the biological compartment (electroporation). Electroporation can be caused with the aim of removing material from or introducing materials into the biological compartment or as a precursor to electrofusion of multiple biological compartments.

In various aspects, an ion enrichment zone and zero electric field strength at the BPE can be used to attract a biological compartment by nDEP. Subsequently, the electric field conditions can be altered to replace the ion enrichment zone with an ion depletion zone. This protocol can result in enhanced trapping of the biological compartment and repulsion of (or prevention of trapping) further biological compartments. Ion depletion surrounding the biological compartment can lead to increased osmotic pressure leading to swelling (fluid uptake) of the biological compartment. Swelling separately or in combination with locally increased electric field strength can cause lysis (catastrophic breakdown of the lipid membrane) of the biological compartment. In some aspects, after lysis, the contents of the biological compartment can be transported electrokinetically into a constriction (e.g., a narrow side channel or series of pores) or a entrapment structure and isolated for storage, processing, or analysis as described herein.

FIGS. 17A through 17D illustrate a device 1700 for lysis of biological compartments. Referring to FIG. 17A, a BPE 1702 can be in contact with a first channel 1704 and a second channel 1706 defined by an insulating material 1708 and containing an ionically conductive phase. Similar to the other dual-channel devices described herein, voltages (V₁, V₂ V₃, and V₄) can be applied at the ends of the first and second channels 1704, 1706. One end of the BPE 1702 can be aligned in an entrapment structure (e.g., notch 1710) in the second channel 1706 that can act as the location for capture of an object (e.g., a cell or other biological compartment). The channels 1704, 1706 can also be interconnected by a third channel 1712, which can serve as a conduit for cell contents following cell capture and lysis. The dimensions of the BPE 1702 and channels 1704, 1006 can be similar to the dimensions of the BPE 302 and channels 306, 308 described in reference to FIG. 3A. The third channel 1712 that can connect channels 1704, 1006 can have a width that is at least about 100 nm, at least about 250 nm, at least about 500 nm, at least about 1 μm, at least about 5 μm, at least about 10 μm, at least about 50 μm, or at least about 100 μm. In some aspects, the third channel 1712 can have a smaller width than the first channel 1704 and/or the second channel 1706. The third channel 1712 can have a height that is at least about 100 nm, at least about 250 nm, at least about 500 nm, at least about 1 nm, at least about 5 μm, at least about 10 μm, at least about 50 μm, or at least about 100 μm.

The device 1700 can be used for cell trapping and lysis and transport of cellular contents, or the device can be used for capture and transport of the contents of any other discrete polarizable phase having suitable characteristics. FIG. 17B illustrates a cell 1714 that has been introduced into the second channel 17106 and subsequently trapped in the notch 1710 by dielectrophoretic forces generated by the BPE 1702 as described herein. FIG. 17C illustrates swelling and membrane disruption of the cell 1714 generated by switching the applied voltages to create ion depletion within the segment of the ionically conductive phase near the compartment 1710. FIG. 17D illustrates removal and transport of cell contents 1716 following cell lysis through the third channel 1712 and into the first channel 1704 (e.g., induced by the application of a suitable voltage across the third channel 1712). The cell contents 1716 can be transported into the first channel 1704 for downstream processing and analysis.

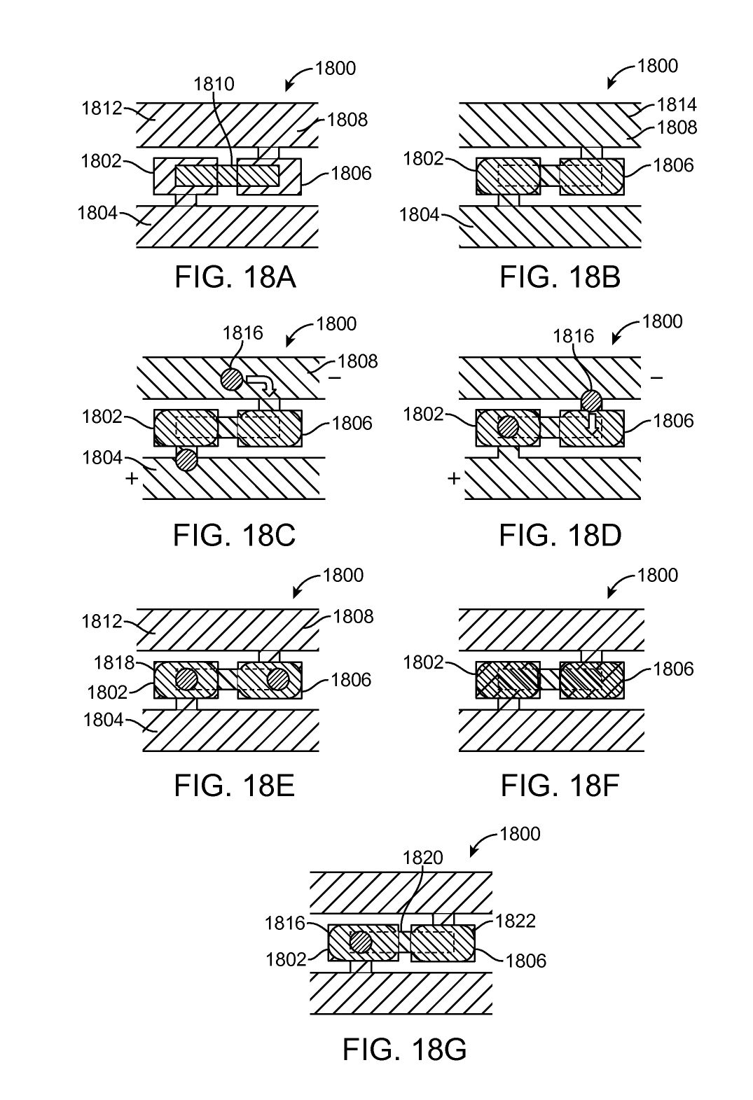

FIGS. 18A through 18G illustrate a device 1800 for trapping and lysis of biological compartments in isolated chambers. In some aspects, the device 1800 is used for PCR analysis of cell lysates. The device 1800 includes a first chamber 1802 connected to a first fluidic channel 1804 and a second chamber 1806 connected to a second fluidic channel 1808. The first chamber 1802 and first fluidic channel 1804 can be fluidically isolated from the second chamber 1806 and second fluidic channel 1808. The device includes a BPE 1810 having one end in the first chamber 1802 and an opposing end in the second chamber 1806.

In some aspects, the device 1800 can be used to perform the following analysis process. First, the device 1800 is primed with an immiscible phase 1812 (e.g., mineral oil) that fills the chambers 1802, 1806 and channels 1804, 1808 (FIG. 18A). Second, the chambers 1802, 1806 and channels 1804, 1808 are filled with an ionically conductive phase 1814 comprising aqueous buffers and reagents (FIG. 18B). Third, a trapping voltage is applied (low dielectrophoretic force) at the channels 1804, 1808 (FIG. 18C). Samples 1816 are flowed in via the channels 1804, 1808 and trapped at the openings of the chambers 1802, 1806. Fourth, the trapping voltage is increased so as to increase the dielectrophoretic force, thereby pulling the trapped samples into the chambers 1802, 1806 (FIG. 18D). Fifth, the channels 1804, 1808 are filled with the immiscible phase 1812 to isolate the samples 1816 in aqueous droplets 1818 (FIG. 18E). Sixth, the samples 1816 are lysed for subsequent analysis (e.g., PCR initiated via infrared illumination as described further herein) (FIG. 18F).

In some aspects, the device 1800 is designed to perform optimally for single-cell analysis, such as single-cell PCR. Features such as device dimensions and the electric field distribution, strength, and frequency can be selected to yield rapid trapping of single cells and sufficient analysis reagents (e.g., PCR amplification reagents). For example, the opening to each chamber 1802, 1806 can be similar in size to the cell diameter to prevent the cell from entering the chamber at low dielectrophoretic force, but sufficiently large to allow the cell to be pulled into the chamber as dielectrophoretic force is increased. Using this strategy, single cells can be held at each opening, thus preventing further cells from being trapped, prior to entering the chamber. Furthermore, the DC field strength can be tuned to create an ion enrichment zone large enough to attract passing cells but sufficiently small to prevent crosstalk between neighboring chambers.

In certain aspects, the first chamber 1802 and second chamber 1806 are fluidically linked by a passage 1820 (FIG. 18G). The first chamber 1802 can be used for trapping sample 1816, while the second chamber 1806 can be used for trapping analysis reagents 1822. The sample 1816 and reagents 1822 can each be trapped in a respective droplet using the techniques described herein. Subsequently, the two droplets can be mixed in order to effect analysis of the sample 1816 via the reagents 1822. The dimensions of the passage 1820 connecting the chambers 1802, 1806 can be selected to optimize the trapping and analysis procedure. For instance, if the passage 1820 is too narrow or too long, the immiscible phase 1812 may become trapped in the passage 1820, preventing the sample and reagent droplets from mixing. If it is too large, however, it may allow the entire cavity (both chambers 1802, 1806) to be filled with sample solution (if it is flowed first, i.e., before the analysis reagent solution).

Some aspects of the present disclosure enable separation of samples by discretizing individual samples into a compartmentalized sample volume, e.g., a droplet. The sample volume can contain the sample of interest as well as the surrounding medium, e.g., the segment of ionically conductive phase near the sample. Such sample volumes can be formed by trapping an object using the methods described herein, then discretizing the sample using a suitable discretization device, e.g., a droplet generator. In certain aspects, each sample volume contains a single sample object, while in other aspects, each sample volume contains multiple sample objects. For example, a single cell can be trapped at a BPE, and then a droplet can be formed that encapsulates the trapped cell. The sample volume can then be further processed and analyzed as desired. Significantly, droplet microfluidics offers unparalleled advantages in high-throughput, small-volume analysis of sample such as single cells. The combination of DEP trapping and droplet encapsulation described herein can be especially powerful because it harnesses these advantages while providing a mechanism for creating and identifying droplets containing individual live cells.

Some aspects of the present disclosure include producing droplets in immiscible fluids. As is well known in the art, a wide variety of immiscible fluids can be combined to produce droplets, e.g., of uniform or varying volumes. As described further herein, the fluids can be combined through a variety of ways, such as by emulsification. For example, an aqueous solution (e.g., water) can be combined with a non-aqueous fluid (e.g., oil) to produce droplets in a sample holder or on a microfluidic chip. Aqueous solutions suitable for use in the present disclosure can include a water-based solution that can further include buffers, salts, and other components generally known to be used in detection assays, such as PCR. Thus, aqueous solutions described herein can include, e.g., primers, nucleotides, and probes. Suitable non-aqueous fluids can include, but are not limited to, an organic phase fluid such as a mineral oil (e.g., light mineral oil), a silicone oil, a fluorinated oil or fluid (e.g., a fluorinated alcohol or Fluorinert), other commercially available materials (e.g., Tegosoft), or a combination thereof.

A variety of fluids or liquids can be used to prepare an emulsion according to the present disclosure. In some aspects, the system includes two or more immiscible fluids, that when mixed under appropriate conditions, separate into a dispersed droplet phase and a continuous carrier phase. For example a first fluid, which will become the dispersed droplet phase, can contain a sample. In some aspects, this first fluid will be an aqueous solution. In some aspects, this first fluid will remain a liquid, in other aspects, it can be, or become, a gel or a solid. In some aspects, this first fluid can have or can form a distinct shell.

Possible aqueous fluids that can be used as one phase of a droplet emulsion include, but are not limited to, various PCR and RT-PCR solutions, isothermal amplification solutions such as for LAMP or NASBA, blood samples, plasma samples, serum samples, solutions that contain cell lysates or secretions or bacterial lysates or secretions, and other biological samples containing proteins, bacteria, viral particles and/or biological compartments or cells (eukaryotic, prokaryotic, or particles thereof) among others. In certain aspects, the aqueous fluids can also contain surfactants or other agents to facilitate desired interactions and/or compatibility with immiscible fluids and/or other materials or interfaces they may come in contact with. In certain aspects, the aqueous solutions loaded on the devices can have cells expressing a malignant phenotype, fetal cells, circulating endothelial cells, tumor cells, cells infected with a virus, cells transfected with a gene of interest, or T-cells or B-cells present in the peripheral blood of subjects afflicted with autoimmune or autoreactive disorders, or other subtypes of immune cells, or rare cells or biological particles (e.g., exosomes, mitochondria) that circulate in peripheral blood or in the lymphatic system or spinal fluids or other body fluids. The cells or biological particles can, in some circumstances, be rare in a sample and the discretization can be used, for example, to spatially isolate the cells, thereby allowing for detection of the rare cells or biological particles.

In some aspects, the second fluid, which would become the continuous phase, will be a fluid that is immiscible with the first fluid. The second fluid is sometimes referred to as an oil, but does not need to be an oil. Potential fluids that can serve as the second fluid include but are not limited to, fluorocarbon based oils, silicon compound based oils, hydrocarbon based oils such as mineral oil and hexadecane, vegetable based oils, ionic liquids, an aqueous phase immiscible with the first aqueous phase, or that forms a physical barrier with the first phase, supercritical fluids, air or other gas phases.

In certain aspects of the present disclosure, the droplets can comprise a fluid interface modification element. Fluid interface modification elements include interface stabilizing or modifying molecules such as, but not limited to, surfactants, lipids, phospholipids, glycolipids, proteins, peptides, nanoparticles, polymers, precipitants, microparticles, or other components. In some aspects, one or more fluid interface modification elements can be present in a fluid that will be comprised in a disperse droplet phase fluid. In other aspects, one or more fluid interface modification elements can be present in a fluid that will be comprised in a continuous carrier phase fluid. In still other aspects one or more fluid interface modification elements can be present in both disperse droplet phase fluids and continuous carrier phase fluids. The fluid interface modification elements present in a fluid that will be comprised in one phase of the emulsion can be the same or different from the fluid interface modification elements present in a fluid that will be comprised in another phase of the emulsion.

In some aspects, of the present disclosure, the fluid interface modification element can be used to prevent coalescence of neighboring emulsion droplets, leading to long-term emulsion stability. In some aspects, fluid interface modification elements can have some other or additional important role, such as providing a biocompatible surface within droplets, which may or may not also contribute to emulsion stability. In some aspects, the components can play a role in controlling transport of components between the fluids or between droplets. Some non-limiting examples of fluid interface modification elements include without limitation ABIL WE 09, ABIL EM90, TEGOSOFT DEC, bovine serum albumin (BSA), sorbitans (e.g., Span 80), polysorbates (e.g., PEG-ylated sorbitan such as TWEEN 20 and TWEEN 80), sodium dodecylsulfate (SDS), 1H,1H,2H,2H-perfluorooctanol (PFO), Triton-X 100, monolein, oleic acid, phospholipids, and Pico-Surf, as well as various fluorinated surfactants, among others.

In some aspects, the emulsion system will consist of a dispersed aqueous phase, containing the sample of interest, surrounded by a continuous oil phase. Other aspects can be variations or modifications of this system, or they can be emulsions of completely different composition or construction. Alternative emulsion systems include multiple emulsions such as water in oil in water (water/oil/water, or w/o/w) emulsions, or oil in water in oil (oil/water/oil, or o/w/o) emulsions. These multiple emulsion systems would then have inner, middle and outer phases. In some aspects, the inner and outer phases can have the same composition. In other aspects, the inner and outer phases can be similar—for example, both aqueous, or both the same oil—but with different sub-components. In other aspects, all three emulsion phases can have different, and sometimes very different, compositions.

In certain aspects, the emulsion system can comprise two immiscible fluids that are both aqueous or both non-aqueous. In further aspects, both emulsion fluids can be oil based where the oils are immiscible with each other. For example, one of the oils can be a hydrocarbon-based oil and the other oil can be a fluorocarbon based oil.

In other emulsion systems, both fluids can be primarily aqueous but still be immiscible with each other. In some aspects, this occurs when the aqueous solutions contain components that phase separate from each other. Some examples of solutes that can be used include, but are not limited to, systems containing dextran, ficoll, methylcellulose, polyethylene glycol (PEG) of varying length, copolymers of polyethylene glycol and polypropylene glycol, polyvinyl alcohol (PVA), polyvinyl pyrrolidone (PVP), Reppal PES, K₃PO₄, sodium citrate, sodium sulfate, Na₂H—PO₄, and K₃PO₄.

In addition to aqueous solutions and non-aqueous fluids, surfactants can also be included to, e.g., improve stability of the droplets and/or to facilitate droplet formation. Suitable surfactants can include, but are not limited to, non-ionic surfactants, ionic surfactants, silicone-based surfactants, fluorinated surfactants or a combination thereof. Non-ionic surfactants can include, for example, sorbitan monostearate (Span 60), octylphenoxyethoxyethanol (Triton X-100), polyoxyethylenesorbitan monooleate (Tween 80) and sorbitan monooleate (Span 80). Silicone-based surfactants can include, for example, ABIL WE 09 surfactant. Other types of surfactants generally well known in the art can similarly be used. In some aspects, the surfactant can be present at a variety of concentrations or ranges of concentrations, such as approximately 0.01%, 0.1%, 0.25%, 0.5%, 1%, 5%, or 10% by weight.

In some aspects, droplet generation is performed at the trapping site, e.g., at or near the BPE. The use of entrapment structures to physically constrain the sample at the trapping site can facilitate the in situ droplet generation described herein. For example, referring again to FIG. 10, an object 1002 trapped within the chamber 1016 can be encapsulated by flowing an immiscible fluid (immiscible with the ionically conductive phase) through the channel 1008, thereby forming a droplet containing the object 1002 and the segment of the ionically conductive phase within the chamber 1016. The droplet can then be displaced from the trapping site, e.g., by convection, introduction of a displacing fluid, application of vacuum, pipetting, electrokinetic transport, or combinations thereof. Similar approaches can be applied to encapsulate samples within droplets using other devices, e.g., the device 1500 of FIGS. 15A and 15B, the device 1600 of FIGS. 16A and 16B, or the device 1800 of FIGS. 18A through 18G.

In certain aspects, modification of device hydrophilicity or hydrophobicity can be used to facilitate droplet generation. Such modification can involve the application of hydrophilic or hydrophobic coatings to various device components. In other aspects, such device components can be fabricated using hydrophilic or hydrophobic materials as desired. It can be advantageous, for instance, for the surfaces (e.g., floor, ceiling, walls) of an entrapment structure used to trap the sample to comprise a hydrophilic material in order to facilitate the formation of aqueous droplets. Alternatively or in combination, the surfaces (e.g., floor, ceiling, walls) of a fluidic channel adjoining the entrapment structure can comprise a hydrophobic material. Referring again to FIG. 10, the inner surface of the chamber 1016 and/or the exposed portion of the BPE 1004 can be hydrophilic, while the surfaces of the channel 1008 (e.g., wall 1014) can be hydrophobic.

In other aspects, droplet generation is performed at a location different from the trapping site, e.g., away from the BPE. This can be accomplished by trapping the sample within a segment of ionically conductive phase comprising an ion depletion zone, then displacing the segment (e.g., via convection of the ionically conductive phase) from the trapping site. If the ion depletion zone segment is interspersed with ion enrichment zone segments, the sample will remain trapped within the ion depletion zone segment due to pDEP forces and can be transported to a desired location, e.g., to a downstream droplet generator. A similar approach can be used to entrap samples within ion enrichment zones via nDEP forces.

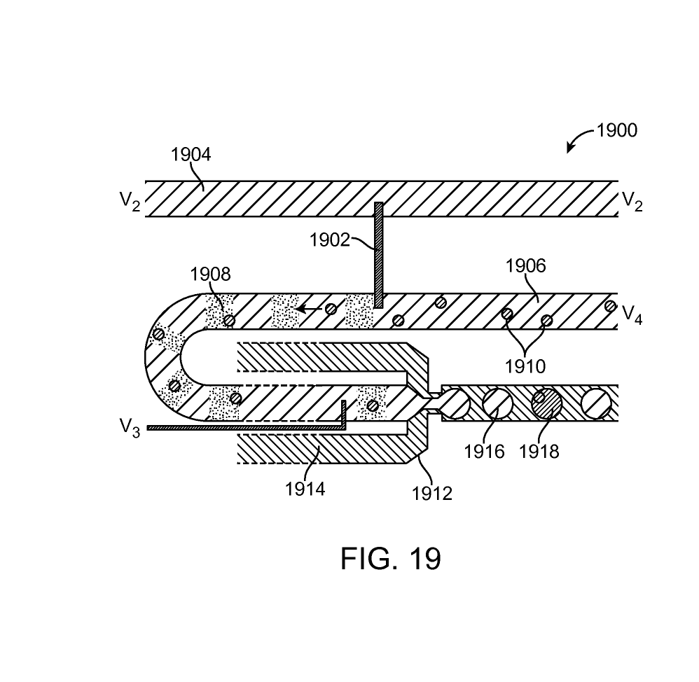

FIG. 19 illustrates a device 1900 for segmenting a sample solution into droplets using a mobile linear array of ion enrichment or depletion zones. The device 1900 includes a BPE 1902 having a first end situated in a first fluidic channel 1904 and a second end situated in a second fluidic channel 1906. The electric field across the ionically conductive phases in the first and second fluidic channels 1904, 1906 can be controlled by voltages (V₁, V₂, V₃, and V₄) applied to the first and second fluidic channels 1904, 1906. A series of segmented ion enrichment zones or ion depletion zones 1908 are introduced into a disordered solution of samples 1910 (e.g., cells) as it flows past the BPE 1902. This can be accomplished through intermittent application of the DC component of the electric field. The samples 1910 migrate via DEP into these ion enriched or depleted segments under the influence of the AC electric field, leading to an ordered 1D array of cells. Subsequently, the ionically conductive phase comprising the segmented ion enrichment or ion depletion zones can be flowed to a downstream droplet generation device comprising inlets 1912 that introduce an immiscible phase 1914. The introduction of the immiscible phase to the ionically conductive phase can result in the formation of droplets 1916. In the depiction of FIG. 19, low conductivity droplets 1918 include sample that is entrapped by pDEP. The advantage of this approach is that the segments containing cells can be distinguished by their conductivity and will occur with predictable periodicity. The segments can be encapsulated by a droplet generator before downstream or off-chip analysis. Therefore, if the periodicity of the depletion zones 1908 and droplets 1916 are similar, the device 1900 can produce droplets 1916 each containing a single sample object. Similarly, droplets 1916 can be sorted based upon their conductivities to yield only those droplets likely to contain sample objects.

The sample processing and analysis techniques described herein can be performed on samples encapsulated within sample volumes such as droplets. Reactions (e.g., amplification) can be carried out in the sample volumes, before or during analysis of the volumes to determine which volumes have undergone reactions (e.g., have amplified product). In certain examples, the volumes (e.g., droplets) can be sized and the number of occupied droplets (e.g., droplets containing a sample as indicated by the presence of a detectable agent) counted. All or just some of the droplets can be analyzed. Analysis can, for example, be achieved by flowing the droplets in a single file through a flow cytometer or similar device, where the size of the droplet can be determined and the presence of amplification can be detected. The size of the droplet can, for example, determined based on the scattering signal from the droplet and the presence of amplification can be indicated by a fluorescence signal from the droplet. Alternatively, the diameter of droplets can be determined by microscopy. In various aspects, droplet diameter and the presence of a detectable agent is detected by an optical detection method. Any detector, or component thereof, that operates by detecting a measureable optical property, such as the presence of light, comprises an optical detector. Examples of optical detectors include, but are not limited to, cameras, photomultiplier tubes, photodiodes and photodiode arrays, and microscopes, and associated components thereof, such as objectives, optical filters, mirrors, and the like.

In certain aspects, the signal detected by an optical detector, or other suitable detector, is processed in order to interpret the signals being measured by the detector. In certain aspects, the measured information is processed by a device, apparatus, or component thereof that stores and/or processes information acquired by a detector, such as, e.g., an optical detector. Examples of an information processor include, but are not limited to, a personal computing device that stores information acquired by a detector, and software running on the personal computing device that processes the information. In other aspects, an information processor or component thereof can be embedded in a detector, such as in a chip embedded in a camera that stores optical information acquired by the camera either permanently or temporarily. In other aspects, an information processor and a detector can be components of a fully integrated device that both acquires and processes optical information to perform a digital assay.

In yet another aspect, the systems can include a computer-readable storage medium for conducting digital measurements. The computer-readable storage medium has stored thereon instructions that, when executed by one or more processors of a computer, cause the computer to: analyze a plurality of droplets to determine a number of droplets in the plurality that contain the detectable agent; and use the number of droplets in the plurality of droplets, the volumes of some or all of the droplets in the plurality and the number of droplets in the second plurality containing one or more detectable agents to determine a concentration of the detectable agent in the sample.

In another aspect, systems are provided for analyzing volumes to detect and calculate information for a given droplet. The system includes one or more processors, and a memory device including instructions executable by the one or more processors. When the instructions are executed by the one or more processors, the system at least receives a user input to analyze volumes (e.g., a plurality of droplets). The system can be configured to carry out aspects of the methods of the present disclosure, such as counting a number of volumes (e.g., droplets), determining volumes of a plurality of droplets in a volume distribution, and using the number of the droplets containing one or more detectable agents to determine a concentration of the detectable agent in the sample. The system also provides data to a user. The data provided to the user can include the concentration of the detectable agent in the sample or a sample concentration.

In some aspects of the present disclosure, the presence of one or more target molecules within a droplet is indicated by an increase of fluorescence in a particular wavelength range. In some aspects, a PCR reaction product indicates the presence of the target molecule by an increase in the fluorescence in a particular wavelength range (indicator fluorescence). In some aspects, a reference agent can be utilized in parallel with the target molecule. According to this aspect, the droplets emit fluorescence (i.e., reference fluorescence) in a wavelength range separate from that of the target molecule regardless of whether the target molecule is present. For a given set of droplets, separate sets of images of the indicator fluorescence and reference fluorescence are obtained and the droplets in each are identified and measured. The indicator and reference fluorescence from a given droplet can be compared. In some aspects, the ratio of the indicator to reference fluorescence can be used to indicate whether that particular droplet contains the target molecule. In other aspects, the absolute intensity of the indicator fluorescence would be sufficient to indicate if the droplet contained target. In some aspects, the average value of the background pixels or a multiple thereof can be subtracted from the pixel intensities within the droplets before the fluorescence intensities of the indicator and reference intensities are compared. By performing this analysis, a list of droplet diameters is obtained, and for each measured droplet, a binary measure is obtained defining whether the droplet is occupied (contains one or more target molecules) or not. The list of droplet sizes and the total number of occupied droplets can then be used to obtain the target concentration of the sample.

There are many possible ways to measure the size, contents, and/or other aspects of droplets in an emulsion while applying the methods of the present disclosure. In some aspects, droplets can be measured optically by an optical detector comprising a flow cytometer. According to this aspect, droplets can flow through a large flow channel where droplet shapes are not distorted and their volumes can be determined by computer software, based on measurements of light scattering patterns acquired by an optical detector, such as a photomultiplier tube, as the droplets pass a source of light excitation. In other aspects droplets can pass through a narrow flow channel where the droplets conform to the channel width. According to this aspect, the volume of the disperse droplets can be determined by using the channel width and the length of the individual droplets in the channel to define their volume.

A variety of signal detection methods can be used according to the present disclosure. In various aspects, the present methods and systems provide for detection of droplet aspects using optical detection methods and optical detectors. In some aspects, the emulsion system can be measured optically by an optical detector comprising a fluorescence microscope and its associated components. Images can be acquired with, for example, a confocal laser scanning microscope, a spinning-disk (Nipkow disk) confocal microscope, or a microscope that uses programmable arrays of mirrors or spatial light modulators to acquire data from multiple focal depths. In other aspects, images can be acquired with an epifluorescent microscope. In some aspects, images acquired with an epifluorescent microscope can be processed subsequently using 3D deconvolution algorithms performed by computer software. In other aspects images can be acquired with a multi-photon microscope, such a two-photon microscope. In other aspects images can be acquired using planar illumination microscopy, Bessel beam microscopy, differential interference contrast microscopy, phase contrast microscopy, bright field imaging, dark field imaging, or oblique illumination. In some aspects, images can be acquired using a combination of the imaging devices and methods listed herein, or any other suitable imaging devices and methods that can reasonably be applied to the present methods.

The method of droplet imaging provides information on both the droplet size and whether the droplet contains a target of interest, which is used according to the present method for the determination of sample concentration. In some aspects, droplet size and signal intensity can be determined based on optical information acquired using confocal fluorescence microscopy. According to this aspect, the emulsion can be stored in a well, chamber, or other container and multiple sets of image stacks can be acquired from it. In some aspects, for each region of interest (ROI) in a given droplet sample, an image stack is collected, consisting of at least two images taken at the same XY-position but different Z-positions (different depths). Droplets that are larger than the spacing in Z will appear in multiple frames at the same XY position, but with different diameters. The image stack enables the determination of various parameters, including the droplet size and the presence of absence of a target analyte in the droplet.

In some aspects, droplet diameters are determined by an information processor based solely on droplets' boundaries determined in the frame of a Z-stack that contains the largest diameter. In other aspects, droplet diameters are determined by an information processor based on droplet boundaries determined at multiple images in a Z stack, and the relative positions of the images in the Z dimension. This method can include an assumption of spherical droplet shape, or some other modified shape, depending on multiple factors including the refractive indices of the two fluids, the relative density of the two fluids and the surface tension.

There are numerous methods to identify and select individual droplets according to the present disclosure. In one aspect, line scans are obtained within the image and, after setting an appropriate threshold level, the diameter of regions of interest are measured. In another aspect, a threshold for each image is chosen, and the areas above the threshold are evaluated as possible single droplets. If the area is sufficiently round (i.e., has an aspect ratio below a selected threshold level), then the area is considered to be a single droplet. A list of droplets is generated for each image.

In some aspects, once droplets in an image have been identified, they are correlated between different frames of the image stack. The largest droplets will appear in more than one image so it is necessary to identify the trail of circles through the frames of the image stack. Droplet correlation can be readily accomplished by using any number of suitable tracking algorithms as would be known to one of ordinary skill in the art. Tracking is generally facilitated by the fact that droplets do not move significantly between frames and because the droplets of interest are fairly large (in pixels). According to the present disclosure, the diameter of a particular droplet can be assumed to be that of the largest circle associated with it in the image stack. Alternatively, a curve can be fit to the circle diameters of a particular droplet and the largest diameter interpolated from that curve. That largest diameter would then be used as the diameter of the droplet. In various aspects of the present disclosure, a plurality of images in the image stack are obtained and used to determine the various parameters of interest for a given droplet, and the droplet itself is not required to undergo additional assaying.

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure provided herein. The upper and lower limits of these smaller ranges can independently be included in the smaller ranges, and are also encompassed within the disclosure, subject to any specifically excluded limit in the stated range. Where the stated range includes one or both of the limits, ranges excluding either or both of those included limits are also included in the disclosure provided herein.

The specific dimensions of any of the apparatuses, devices, systems, and components thereof, of the present disclosure can be readily varied depending upon the intended application, as will be apparent to those of skill in the art in view of the disclosure herein. Moreover, it is understood that the examples and aspects described herein are for illustrative purposes only and that various modifications or changes in light thereof can be suggested to persons skilled in the art and are included within the spirit and purview of this application and scope of the appended claims. Numerous different combinations of aspects described herein are possible, and such combinations are considered part of the present disclosure. In addition, all features discussed in connection with any one aspect herein can be readily adapted for use in other aspects, herein. The use of different terms or reference numerals for similar features in different aspects does not necessarily imply differences other than those expressly set forth. Accordingly, the present disclosure is intended to be described solely by reference to the appended claims, and not limited to the aspects disclosed herein.

As used herein A and/or B encompasses one or more of A or B, and combinations thereof such as A and B.

Unless otherwise specified, the presently described methods and processes can be performed in any order. For example, a method describing steps (a), (b), and (c) can be performed with step (a) first, followed by step (b), and then step (c). Or, the method can be performed in a different order such as, for example, with step (b) first followed by step (c) and then step (a). Furthermore, those steps can be performed simultaneously or separately unless otherwise specified with particularity.

While preferred aspects of the present disclosure have been shown and described herein, it is to be understood that the disclosure is not limited to the particular aspects of the disclosure described below, as variations of the particular aspects can be made and still fall within the scope of the appended claims. It is also to be understood that the terminology employed is for the purpose of describing particular aspects of the disclosure, and is not intended to be limiting. Instead, the scope of the present disclosure is established by the appended claims. In this specification and the appended claims, the singular forms “a,” “an” and “the” include plural reference unless the context clearly dictates otherwise.

All publications, patents, and patent applications mentioned in this specification are herein incorporated by reference to the same extent as if each individual publication, patent, or patent application was specifically and individually indicated to be incorporated by reference.

The specific dimensions of any of the apparatuses, devices, systems, and components thereof, of the present disclosure can be readily varied depending upon the intended application, as will be apparent to those of skill in the art in view of the disclosure herein. Moreover, it is understood that the examples and aspects described herein are for illustrative purposes only and that various modifications or changes in light thereof can be suggested to persons skilled in the art and are included within the spirit and purview of this application and scope of the appended claims. Numerous different combinations of aspects described herein are possible, and such combinations are considered part of the present disclosure. In addition, all features discussed in connection with any one aspect herein can be readily adapted for use in other aspects herein. The use of different terms or reference numerals for similar features in different aspects does not necessarily imply differences other than those expressly set forth. Accordingly, the present disclosure is intended to be described solely by reference to the appended claims, and not limited to the aspects disclosed herein.

### EXEMPLARY ASPECTS

### Example 1

### Dielectrophoretic Manipulation of Cells Using a Dual-Channel BPE Device

This example provides exemplary methods for dielectrophoretic manipulation of cells using a dual-channel BPE microfluidic device similar to the embodiments of FIG. 7.

A microfluidic device comprised of two separate microfluidic channels in electrochemical contact with a BPE was used to attract, trap, and swell, lyse, or release biological cells in the presence of a flowing aqueous buffered solution. The forces employed to trap the cells were dielectrophoretic in nature and were generated via the application of an AC field with a DC offset. The field was applied in such a manner that the region of the solution in direct contact with the BPE experienced zero electric field strength (E=0). Faradaic reactions at the BPE were used to alter the ionic strength in the vicinity of the electrode. An increase in the ionic strength aided in attraction of one or several biological cells to the trapping point (where E=0, on the surface of the BPE) depending on trapping time and the concentration of biological cells in the aqueous solution. A subsequent decrease in the ionic strength in this region led to swelling, electroporation, and lysis of a captured cell. Ceasing to apply the electric field led to release of captured cells.

A PDMS/glass hybrid microdevice was fabricated using photolithographic procedures. First, a glass substrate (25 mm×75 mm×1 mm) coated on one side with 100 nm-thick gold was spin-coated with a positive photoresist (˜7 μm thick). Then, the photoresist was patterned via standard photolithographic procedures to cover a portion of the gold/glass substrate with the desired electrode dimensions (3 mm-long×100 μm-wide rectangle with a tapered tip). Next, the substrate and patterned photoresist were immersed in gold etchant until only the photoresist-masked gold was retained on the glass substrate. Finally, the substrate was then rinsed with distilled water and dried with nitrogen gas.

To define the channels, PDMS (approx. 5 mm thick) was caste on an SU-8 master on a Si-wafer substrate and cured at 70° C. for at least 2 hrs. The side-by-side channels were 15 mm long, 90 μm wide, 18 μm tall and separated by 3 mm. Inlet and outlet reservoirs (3 mm diameter) were punched in the PDMS monolith. The PDMS microchannels and gold/glass substrate were washed with ethanol and dried with nitrogen. Third, the microchannels were visualized under a microscope and aligned such that the BPE was centered along the length of both microchannels and contacted both microchannels at a 90° angle. A droplet of ethanol was placed on the gold/glass substrate prior to alignment to allow the PDMS monolith to glide over the substrate for easier alignment. After aligning, the remaining ethanol was removed by evaporative drying in a 70° C. oven. This drying procedure resulted in reversible bonding of the PDMS to the glass substrate. Finally, the microchannels were coated to prevent cells from sticking to the channel surface. The channels were coated by filling them with 3 μM ethylene oxide—propylene oxide block copolymer in 100 mM Tris buffer (pH 8.0), covering the reservoirs, and storing the device overnight at 4° C. The channels were rinsed with fresh 100 mM Tris buffer (pH 8.0) to remove excess coating agent prior to cell trapping.

The biological cells employed in the present example were BaF3 Mouse pro-B cells cultured by standard protocol in RPMI-1640 cell culture media supplemented with 10% fetal bovine serum and 1% penicillin streptomycin. Prior to trapping experiments, the cells were pelleted (centrifugation at 2300 rpm) and resuspended in 100 mM Tris buffer (pH 8.0). This pelleting and resuspension was repeated to ensure removal of residual cell culture media.

Combined electrophoretic and dielectrophoretic trapping of a single B-cell or multiple cells proceeded as follows. First, the PDMS/glass device was taped to a reflective backing (Si-wafer) to aid in visualization by a wide-field microscope (top-lit). The solution in the inlet and outlet of one channel (trapping channel) was removed and replaced with unequal volumes (˜20 μL inlet and ˜15 μL outlet) of the B-cell solution (˜1×10⁶ cells/mL). The height differential between the solutions in the two reservoirs established and maintained slow pressure driven flow of B-cell solution through the channel throughout the duration of the trapping experiment.

Pt-wire driving electrodes were dipped in all four reservoirs such that the reservoirs at the inlet and outlet of each individual-channel shared one wire. The driving voltage was applied using a waveform generator. Images were collected using an in-house program developed using LabView software.

Cells were verified to be alive or dead following trapping and manipulation via staining with trypan blue for 5 min. A 0.4% solution of trypan blue was diluted 10× in 100 mM Tris buffer (pH 8.0) and loaded into the channel. Trypan blue loading was achieved by replacing the complete volume of solution in one reservoir of the trapping channel with an equal volume of trypan blue solution. This procedure maintained the previously established volume difference between the inlet and outlet reservoirs and allowed slow pressure driven flow of the trypan blue solution into the channel. Cell viability was determined after 5 min based on the staining of cell debris as a control for dead cells.

FIG. 20A shows the anodic tip of the BPE extending into the anodic channel of the microfluidic device. The tip of the BPE was aligned with an approximately 20 nm×20 nm notch in the PDMS wall. In the cell trapping experiment shown in FIG. 20A through 20C, first, both channels of the device were rinsed with 100 mM Tris (pH 8.0), and then with the same buffer solution containing ˜1×106 mouse pro-B cells/mL. An excess of 5 μL, of this B-cell solution was added to one reservoir of the anodic channel (right side, FIG. 20A) to establish slow flow of the solution in the microchannel. Next, as a B-cell approached the BPE tip (within about 500 μm), the trapping voltage was turned on. The applied field had an AC component at 1 kHz and 10 V peak-to-peak with a 5 V DC offset such that this tip of the BPE acted as the anode. The water oxidation led to a charge enrichment region around the BPE tip. A single B-cell was observed to accelerate towards and then be trapped at the BPE anode (FIG. 20B). The B-cell was trapped at the BPE tip for the remaining duration that the applied field was maintained. After 1 min, the applied field was turned off, and the cell was released as shown in FIG. 20C. This experiment demonstrates trapping and release of a single cell.

In a separate experiment (results not shown), a cell trapped using the same experimental conditions was held trapped at the BPE (i.e., applied electric field was maintained) for an additional 5 min while trypan blue staining was performed. The trapped cell did not stain after 5 min, while a nearby piece of cellular debris (used as a positive control for cell death) was stained.

FIG. 21A through 21C show combined electrophoretic and dielectrophoretic trapping of multiple B-cells at the end of a single BPE that is extended farther into the channel. In this experiment, the same experimental procedure was used as for trapping a single cell, except that the concentration of B-cells filling the channel was higher (˜3×106 cells/mL), the DC offset was ramped (0.5 V every 30 s) to a trapping voltage of 4.0 V, and the trapping voltage was held for 2 min and 15 s. Images were taken after 30 s (FIG. 21A), 1 min 45 s (FIG. 21B), and 2 min 15 s (FIG. 21C) after starting to apply the trapping voltage. As the trapping voltage was ramped, no cells were trapped until 4.0 V DC offset was reached, indicating that the minimum voltage required for trapping cells was about 4.0 V.

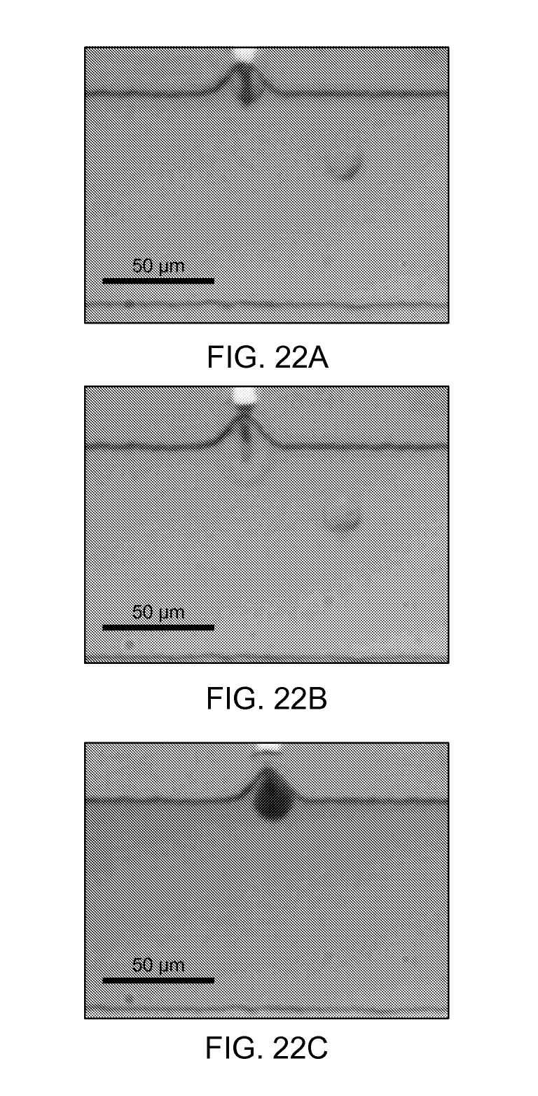

FIGS. 22A through 22C show swelling, and lysis (disruption of membrane integrity) of a single B-cell. The experimental procedure leading to cell trapping was the same as that employed in the experiment shown in FIG. 20B, with the exception that the electrode tip was aligned so that it was recessed in a triangular notch (approx. 15 μm wide and 10 μm tall). The trapped cell is shown in FIG. 22A. The initial trapping conditions were maintained for 1 min, and then the sign of the DC offset was switched from 5 V to −5 V, therefore causing this end of the BPE to act as a cathode. The reduction of water to produce hydroxide ion at the BPE and the subsequent neutralization of the buffer cation (TrisH+) led to the formation of an ion depletion zone around the BPE. The reactions at the BPE led to swelling of the trapped cell (compare FIGS. 22A and 22B). After cell swelling, the applied voltage was switched back to the initial trapping conditions, and trypan blue staining was performed as described above. Dark staining of the cell (FIG. 22C) confirmed that the cell membrane had been disrupted.

### Example 2

### nDEP Manipulation of Cells at a BPE Cathode and a BPE Anode

This example describes an exemplary process for nDEP attraction and repulsion of B-cells from both a BPE cathode and anode. The direction of nDEP force in each case was determined by whether conditions for FIE or FID at the BPE were chosen in the experimental design. The results demonstrate that FIE and FID zones generated by BPEs can be exploited to shape and extend the electric field gradients responsible for dielectrophoretic (DEP) force. First, nDEP repulsion of B-cells from a BPE in the absence of faradaic reactions (i.e., no DC field component) is demonstrated. It is then shown that FIE at either the BPE anode or cathode leads to nDEP attraction that increases with increased AC field strength. These results are contrasted with nDEP repulsion of B-cells from an FID zone.

The RPMI 1640 media employed for cell culture was purchased from American Type Culture Collection (ATCC) (Manassas, Va.). Ethylene glycol-propylene glycol block copolymer (Pluronic® F108), bovine serum albumin (BSA) (≧98% purity), and 1.0 M Tris-HCl stock solution were obtained from Sigma-Aldrich, Inc. (St. Louis, Mo.). The silicone elastomer and curing agent (Sylgard 184) used to prepare the poly(dimethylsiloxane) (PDMS) microfluidic devices were obtained from K. R. Anderson, Inc. (Morgan Hill, Calif.). All other chemicals were reagent grade and purchased from Fisher Scientific (Thermo Fisher Scientific, Inc., Waltham, Mass.) including sodium phosphate (mono- and dibasic), sucrose, and dextrose (D-glucose). All dilutions were carried out with Milli-Q water (18.0 MΩ·cm). DEP buffers were comprised of 8.0% sucrose, 0.3% dextrose, and 0.1% BSA in either 10 mM Tris (pH 8.1) or 10 mM phosphate (pH 7.2) buffer.

Mouse pro-B BaF3 B-cells were obtained from ATCC. These B-cells were cultured in RPMI 1640 supplemented with 1% pen-strep and 10% fetal bovine serum at 37° C. and 5% CO₂. The cells were sub-cultured every 3-4 days such that the concentration of cells did not exceed 1×10⁶ cells/mL. In preparation for DEP experiments, ˜1×10⁶ cells were pelleted by centrifugation followed by resuspension in 5 mL of the desired DEP buffer. This process was repeated one additional time to ensure cell culture medium components were removed.

PDMS/glass hybrid microfluidic devices with embedded Au BPEs were fabricated using standard photolithographic techniques. Briefly, 1 mm-thick glass slides coated with 100 nm Au (no binding layer) were photolithographically patterned using SPR220-7.0 photoresist followed by wet-etching the Au in a 10% KI and 2.5% I₂ solution. The remaining photoresist was then dissolved with acetone. PDMS microchannels were molded by pouring precursor onto an SU-8 master and curing at 70° C. for 2 hours. 4 mm-diameter reservoirs were punched at both ends of each microchannel. The PDMS and Au-on-glass substrates were aligned and irreversibly sealed by the following process. First, both substrates were exposed to an O₂ plasma (plasma cleaner, Harrick Scientific, Ithaca, N.Y.) for 1 min. Second, a drop of ethanol was applied to the glass substrate. Third, the PDMS monoli99th was put in contact with the glass substrate and aligned under a microscope. Then, the device was baked at 70° C. for 1 hour to drive off ethanol. Finally, the device was filled with 3 μM Pluronic in either 10 mM Tris (pH 8.0) or 10 mM phosphate (pH 7.2) buffer selected to match the type of DEP buffer to be employed. The device was covered with parafilm, and incubated at 4° C. overnight (at least 18 hrs). Pluronic coating served to dampen electroosmotic flow.

The device dimensions were as follows. Dual parallel microchannels were each 4.0 mm long×20 μm tall×60 μm wide and separated by 400 μm. The channel inlets were tapered with a 53° angle leading to 4.0 mm-diameter reservoirs. The ceiling of the inlets was supported with diamond-shaped pillars (100 μm×40 μm). This inlet geometry was designed to facilitate unimpeded introduction of cells into the microchannels. At the center of one microchannel (the DEP channel 1008 of FIG. 10), there was a 30 μm×30 μm side chamber, which was aligned to the BPE tip. The exposed BPE tip was approximately 30 μm wide×30 μm long (defined by chamber). The auxiliary end of the BPE extended across the auxiliary channel (channel 1006 of FIG. 10) and was 15 μm wide.

The combined AC/DC electric field was applied to four Pt wires dipped in the device reservoirs (V₁, V₂, V₃, and V₄ of FIG. 10) using a Hewlett-Packard 33120A waveform generator (Hewlett-Packard, Palo Alto, Calif.) and Kepco Model BOP 1000M amplifier (Kepco, Inc., Flushing, N.Y.). The AC field frequency was maintained at 1.8 kHz, at which the Clausius-Mossotti factor is −0.5 (maximum nDEP force) for B-cells under the conditions employed here. Prior to a DEP experiment, each microfluidic channel was rinsed with the appropriate DEP buffer (as indicated below) for 1 min at 3 psi. The reservoirs were then filled with DEP buffer containing 2×10⁵ B-cells/mL.

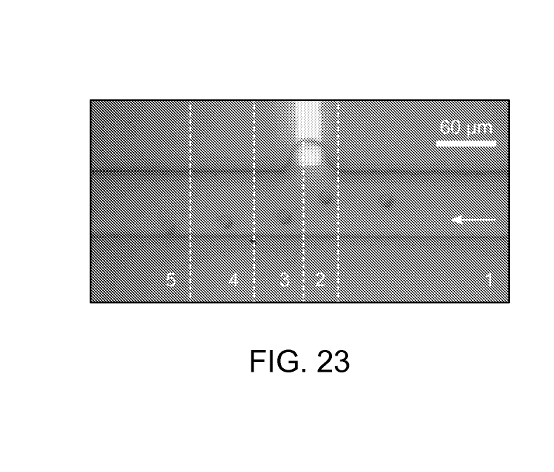

FIG. 23 demonstrates that a B-cell undergoes nDEP repulsion from a BPE tip in an AC-only electric field. In this experiment, the DEP channel (channel 1008 of FIG. 10) was rinsed with DEP buffer (8.0% sucrose, 0.3% dextrose, and 0.1% BSA in 10 mM Tris (pH 8.0)) and then it was filled with the same DEP buffer containing 2×10⁶ B-cells/mL. The auxiliary channel was rinsed and filled with 10 mM NaCl as an electrolyte. Flow (right to left, FIG. 23) was established in the DEP channel by introducing a solution height differential in the reservoirs at its ends such that the average linear flow velocity, V_{avg}=20 μm/s. An AC voltage of 64 Vpp at 1.8 kHz was applied to the left-hand reservoir of the DEP channel (V₃, FIG. 10), and the remaining three reservoirs were grounded. Under these conditions, the spatially averaged root-mean-square (RMS) electric field strength along the microchannel was E_{RMS,avg}=5.7 kV/m. As the cell approached the BPE, E_{RMS,avg} was increased from 5.7 kV/m at t=0 s (slice 1) to 17.7 kV/m at t=5 s (slice 3). The cell was briefly attracted toward the BPE and then repelled by nDEP from the locally high electric field around the BPE tip. This result is significant because it establishes that: (1) these AC field strengths are sufficient to exert significant nDEP force; and (2) the electric field strength around the BPE is a local maximum in the absence of faradaic current and FIE. This experiment establishes that an AC field alone results in nDEP repulsion of B-cells from the BPE tip.

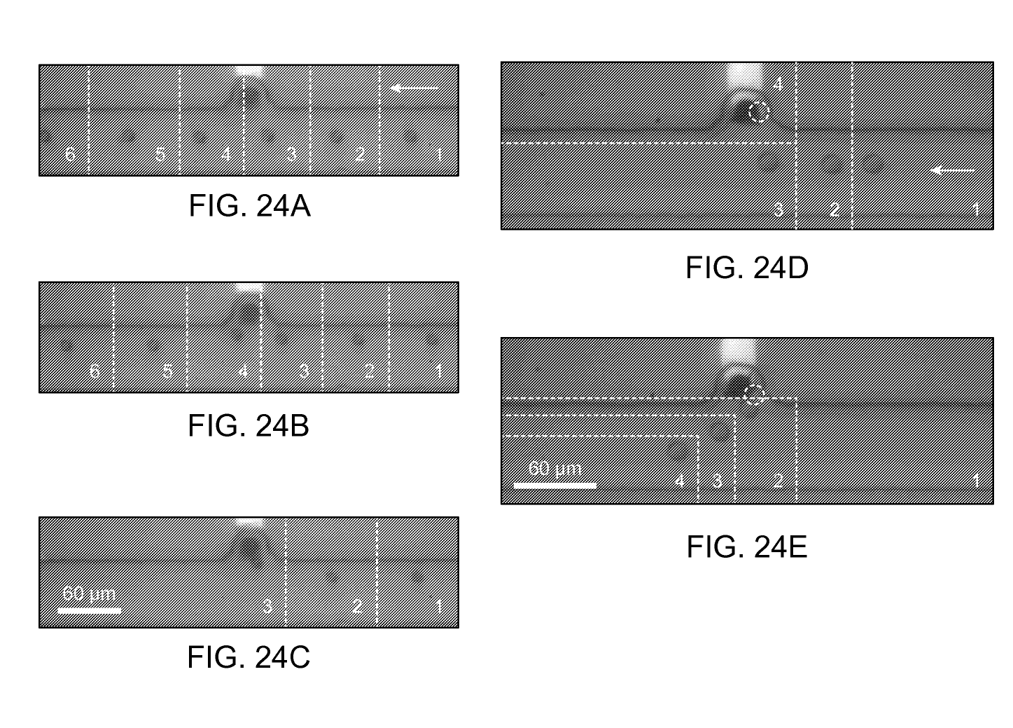

FIGS. 24A through 24E demonstrate nDEP attraction to the BPE with the addition of a DC offset. The DC field can drive faradaic current (i_{BPE}) leading to an FIE zone at either a BPE anode or a BPE cathode. Due to the negative charge of the cell membrane, in these two cases, DEP force works with and against electrophoretic (EP) force, respectively. First, nDEP attraction of a B-cell to an FIE zone at the BPE anode in Tris DEP buffer was examined (FIGS. 24A through 24C). In this device, nDEP cell trapping proceeded at the BPE anode as follows. First, the channels were rinsed and filled as described in the previous subsection. Then, flow was established as before such that V_{avg}=65 μm/s. An AC field with a negative DC offset was applied at V₃ versus ground (V₁, V₂, and V₄) such that E_{RMS,avg}=5.7 kV/m AC and E_{DC,avg}=0.75 kV/m DC. FIG. 24A shows the resulting cell trajectory in 1 s slices. Under these conditions, the EP force exerted by the BPE anode was insufficient to attract and trap the B-cells. However, as the AC field strength was increased (FIGS. 24A through 24C; E_{RMS,avg}=5.7 kV/m, 13.3 kV/m, and 17.7 kV/m, respectively) cells were increasingly attracted and finally trapped. This finding is significant because nDEP attraction toward the BPE indicates that the electric field is depressed around the BPE by FIE.

This result is attributed to an averaged axial electric field profile like that shown in FIG. 8B (dashed line indicating ‘Enrichment’) caused by the progression of the oxidation reaction described by eq. 1 leading to the accumulation of ionic species around the BPE. This ion enrichment zone decreases E locally. Importantly, although E is zero above the BPE whenever iBPE is non-zero (solid and dashed lines, FIG. 8B), cells can only be attracted to this region after an FIE zone forms.

Similarly, nDEP trapping of a B-cell was carried out at the BPE cathode (FIGS. 24D and 24E). In this case, a similar device was filled with 10 mM phosphate (pH 7.2) in 8% sucrose, 0.3% dextrose, and 0.1% BSA (phosphate DEP buffer). An AC field with a positive DC offset was applied at V₃ such that E_{RMS,avg}=5.7 kV/m AC and E_{DC,avg}=1.5 kV/m DC. Water reduction followed by deprotonation of phosphate species led to ion enrichment around the BPE tip. As the AC field strength was increased gradually from 5.7 kV/m to 28.3 kV/m, the cell was pulled into the chamber by nDEP (FIG. 24D, 4 s/slice), and as the AC field was returned to 5.7 kV/m, the cell was expelled from the chamber (FIG. 24E, 2 s/slice). This result is significant for two reasons. First, as in the previous experiment, this result demonstrates that faradaic current leading to FIE sufficiently decreases the local electric field around the BPE to reverse the role of nDEP from repulsion to attraction. Second, in this case, the cell was trapped by nDEP force despite electrostatic repulsion of the negatively charged cell from the BPE cathode. This is demonstrated by the immediate expulsion of the cell from the chamber once the AC field strength was decreased (FIG. 24E).

Importantly, this result has been repeated with the BPE misaligned from the chamber such that the two features are laterally separated by 50 μm and the BPE extends 15 μm into the channel (results not shown). In this control experiment, regardless of the direction of flow, cells favored trapping at the BPE rather than the chamber. This result verifies that the zero electric field directly above the BPE and FIE depression of the surrounding field are the primary mechanisms responsible for cell trapping.

Furthermore, a control was performed with no BPE (results not shown). While the electric field in an empty chamber (no BPE) is lower than that in the microchannel, at AC field strengths up to E_{avg}=28.3 kV/m, cells are only weakly attracted to the chamber and are only drawn into it under stopped-flow conditions.

Just as crucial as FIE is to the understanding of the impact of local conductivity gradients on F_{DEP} is an examination of the FID regime. The enhanced local electric field strength associated with depletion can lead to enhanced EP exclusion of particles, thus, causing the delineation of DEP and EP forces in the FID zone. To separately interrogate the role of nDEP in cell repulsion from an FID zone, the AC field contribution was once again increased while maintaining a constant DC component. In this experiment, the device was prepared with 10 mM Tris DEP buffer (DEP channel) and 10 mM NaCl channel (auxiliary channel) as described previously. A flow rate of V_{avg}=85 μm/s (left to right) was established in the channel. An AC field with a positive DC offset was applied at V₃ such that E_{RMS,avg}=0.7 kV/m AC and E_{DC,avg}=1.25 kV/m DC. Water reduction at the BPE cathode followed by neutralization of TrisH+ ions (eqs. 3 and 4) led to ion depletion around the BPE tip.

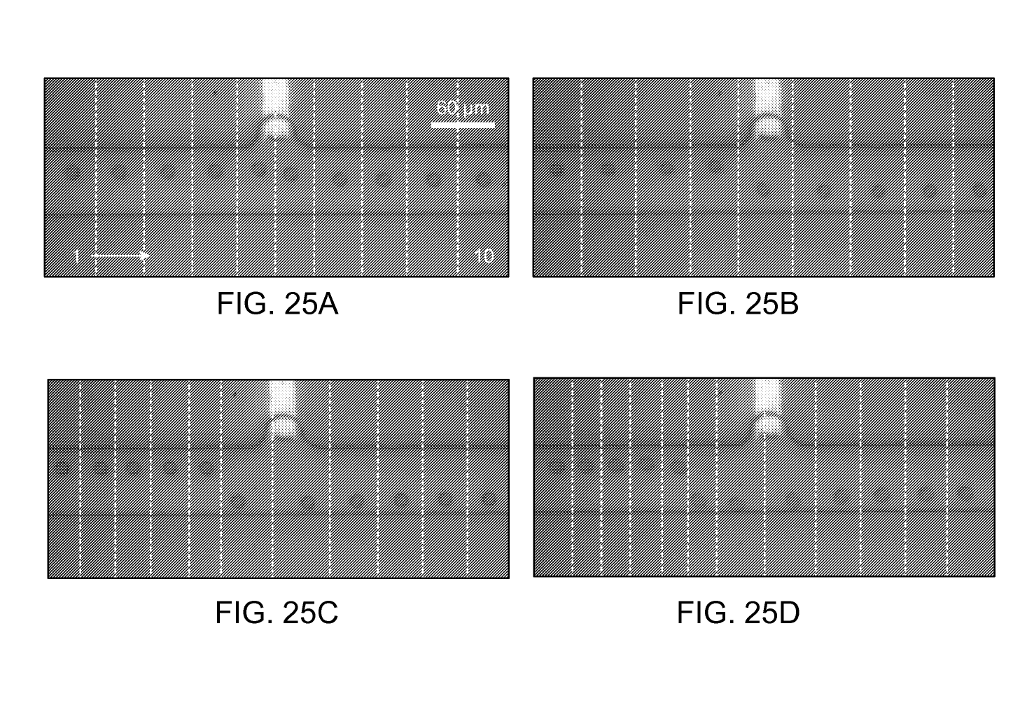

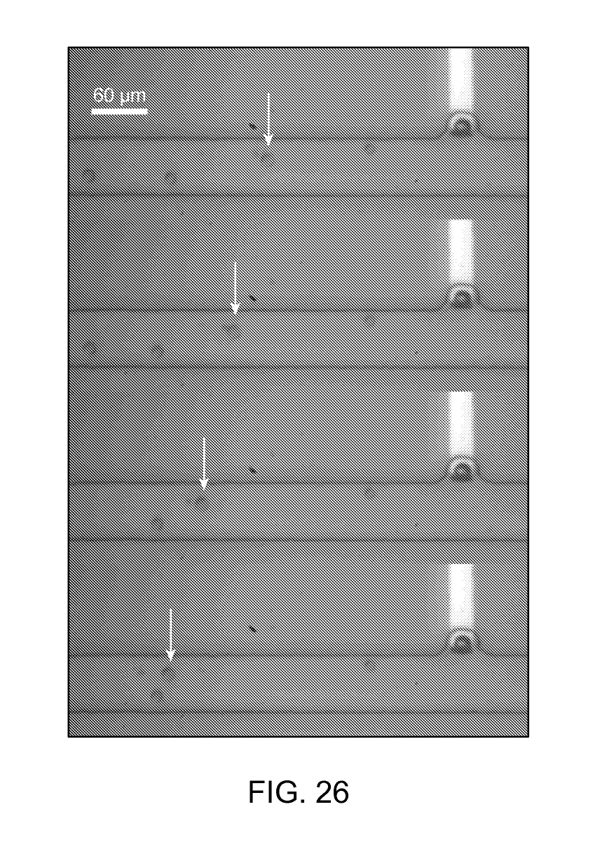

FIGS. 25A through 25D (0.5 s/slice) show increasing degrees of nDEP repulsion of a B-cell from an from the resulting FID zone as the AC field strength was increased from E_{RMS,avg}=0.57 kV/m to 6.13 kV/m, 7.95 kV/m, and then, 10.25 kV/m, respectively. Significantly, by simply changing the identity of the DEP buffer from phosphate, which creates an FIE zone in the presence of OFF, to Tris, which is neutralized under the same conditions, cells go from being pulled into the chamber (FIG. 24D) to colliding with the opposing channel wall (FIG. 25D). Furthermore, this demonstrates that the causative force is dielectrophoretic. nDEP repulsion of cells from an FID zone formed at the BPE anode in phosphate DEP buffer was also performed (FIG. 26). The nDEP force competes with electrophoretic attraction. In FIG. 26, a cell near the BPE tip remains stationary, while a cell farther from the BPE tip (arrows) is repelled.

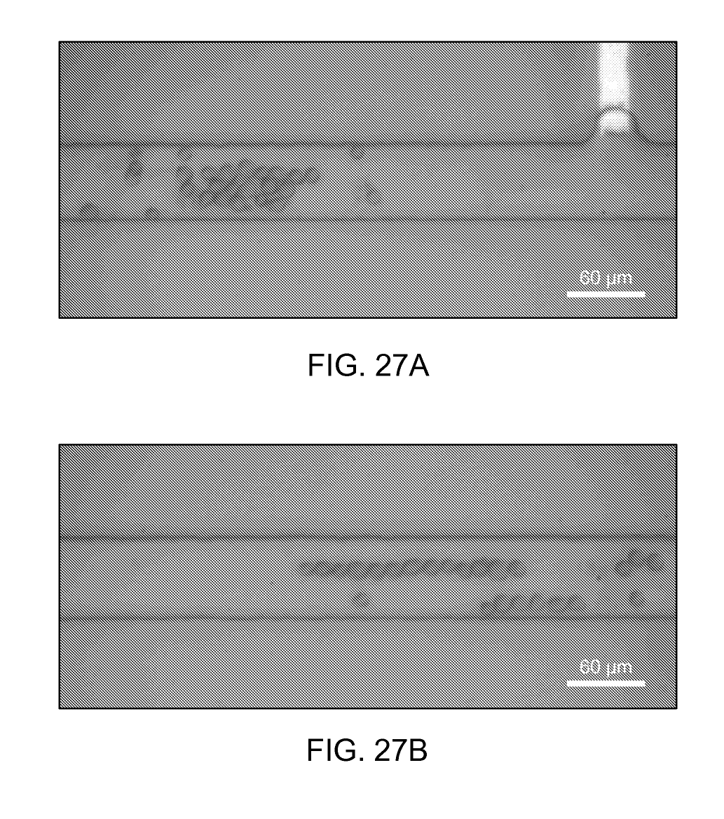

In the previous experiment, the flow rate and faradaic reaction rate were selected such that cells could circumvent the FID zone. FIGS. 27A and 27B show cells repelled by a stronger and larger FID zone (E_{DC,avg}=2.5 kV/m). It is important to note that EP repulsion of the negatively charged cells by the enhanced local electric field around the BPE cathode likely plays a significant role at this DC field strength. At low AC field strength (E_{RMS,avg}=0.57 kV/m, FIG. 27A), cells were impeded and accumulated along the electric field gradient formed by the FID zone where the force of electrophoresis and opposing fluid flow on the cells balanced. This effect has been observed with a DC-only field (results not shown). When the AC field was subsequently increased to E_{RMS,avg}=10.25 kV/m, the additional nDEP force transported the cells to a new balance point >450 μm from the BPE (FIG. 27B). Pearl chaining was observed under these conditions due to the high AC field strength and fixed location of the cells. Importantly, the FID zone extends the reach of DEP force to several hundred microns from the BPE. Given a larger channel width, it is anticipated that cells would be able to circumvent the large FID zone, albeit at several hundred microns from the BPE. These results also demonstrate the many roles of the DC field component: activation of the BPE (i_{BRE} t 0), control of FIE/FID zone size, and EP force. Therefore, the strength of the DC field is critical to DEP outcomes in a BPE-based device.

These experiments demonstrated both nDEP attraction and repulsion of biological cells from each a BPE cathode and anode including single cell sequestration in a side chamber. Furthermore, it was shown that the direction, magnitude and extent of nDEP force can be controlled via faradaic reactions at the BPE, which impact the local conductivity of the DEP medium through the formation of FIE and FID zones.

### Example 3

### Analysis of nDEP Forces Near a BPE

This example describes an analysis of dielectrophoretic forces involved in nDEP repulsion of a cell from a BPE tip.

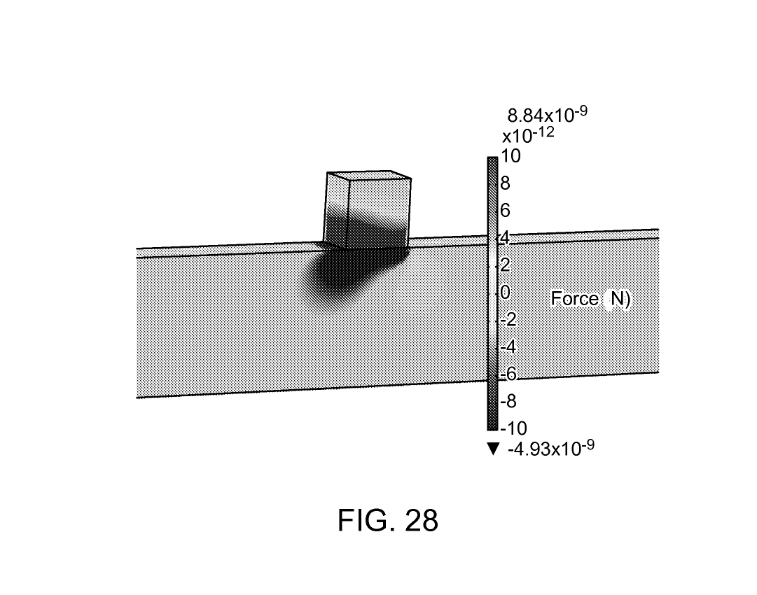

The analysis is performed using COMSOL Multiphysics version 4.4 software. The geometry employed for the analysis is a 500 μm-long segment of a 20 μm-tall by 60 nm-wide microchannel. The microchannel has a 30 μm-long×30 nm-wide×20 μm-tall chamber embedded in the wall at the center of the microchannel segment. Simulation parameters are as follows. The aqueous medium is modeled as a non-solid with relative permittivity of Eᵣ=80. The channel walls (boundaries) are uncharged, to model a Pluronic-coated microchannel. The boundary defining the floor of the chamber is assigned an electric potential of 3.125 V. The inlet (left of FIG. 28) and outlet (right of FIG. 28) are assigned 12.5 V and 0.0 V, respectively.

The 3D geometry is divided into finite elements with a free tetrahedral mesh having a maximum element size of 2.5 μm. A stationary linear solver is used to determine the distribution of electric potential based on charge conservation. Finally, the resulting distribution of electric potential is used to derive the plot of the y-component of dielectrophoretic force using the following equation:

F_{DEP}=2πr³E₀εᵣRe[K(ω)]×\sqrt{(Eₓ²+E_{y}²+E_{z}²)}×d(\sqrt{(Eₓ²+E_{y}²+E_{z}²)})/dy

Here, Re[K(ω)]=−0.5, r=10 nm, ε₀ is the vacuum permittivity, and Eₙ is the magnitude of the electric field along the nth axis.

This analysis implies a DC electric field with no charge migration, and the real system comprises an AC electric field with mobile charged species. However, under AC electric field conditions, there should be no electromigration or accumulation of charged species. Therefore, a DC electric field with no charge migration accurately approximates a time-averaged or root-mean-square (RMS) AC electric field.

FIG. 28 shows the y-component of F_{DEP} (F_{DEP,y}) surrounding a BPE in the xy plane located at z=5 nm above the BPE and channel floor. In this analysis, i_{BPE}=0 and E_{RMS,avg}=25 kV/m. Negative values of F_{DEP,y} indicate nDEP repulsion (in the negative direction on the y-axis). The magnitude of F_{DEP} ranges from 320 pN to 760 pN. At several cell diameters from the BPE, F_{DEP,y} is nearer to 10 pN, which is consistent with typical F_{DEP} magnitudes 10-20 nm from an electrode surface. Significantly, this analysis demonstrates the trajectory of a B-cell as it traverses the channel from right to left. There is weak (several pN) attraction of the cell (positive y-direction) to the right of the BPE followed by further-reaching repulsive forces.

### Example 4

### Determination of the Distribution of Genetic Mutations in Acute Myeloid Leukemia (AML) Cell Populations

This example describes an exemplary process for screening clinical AML samples using a BPE array-based system for parallel manipulation of cells. This process can be used for genetic analysis of tens of thousands of individual cells on a simple microfluidic platform. Using this platform, single cells can be partitioned into isolated sample chambers to enhance the sensitivity and specificity of subsequent PCR.

The process provided in this example can be used to understand the role of genetic heterogeneity in the relapse of leukemia. Despite advances in chemotherapy, many cancer patients experience remission only to relapse. Recent studies have shown that the incidence of relapse can be correlated to the presence of minimal residual disease (MRD), which is characterized by a very low number of disease cells that survive chemotherapy. One hypothesis explaining the cause of MRD is that populations of leukemia cells are genetically heterogeneous (despite clonal growth patterns) and therefore respond differently to chemotherapy, leaving behind resistant cells and resulting ultimately in relapse. For example, patients with acute myeloid leukemia (AML) harboring a mutation in the FLT3 gene will occasionally relapse without the FLT3 mutation, and vice-versa. In addition, patients with Ph chromosome positive leukemia often relapse with point mutations in the Bcr-Abl oncogene that renders them insensitive to the tyrosine kinase inhibitor imatinib; sensitive methods have detected the point mutation clone prior to therapy, suggesting the rare clone emerges under selective pressure. However, it is unclear if this clonal selection is the rarity or the rule, and current technologies are unable to characterize the clonal variability in a tumor and study the patterns of clonal selection and resistance that may occur during treatment. The approach described herein can confirm or deny this hypothesis by providing a picture of cell-to-cell differences in genetic mutations. Furthermore, description of the statistical distribution of genotypes across a population of a patient's leukemia cells prior to chemotherapy may help predict the outcome of chemotherapy and lead to better selection of chemotherapeutic agents. Ensemble amplification of targets (from the entire population) by PCR may be inappropriate for this purpose because resistant cells may make up a minute fraction of the total cell population. Information from single cells of rare genetic composition may be lost against the background of majority cells. Conversely, recent single cell PCR techniques may be too low-throughput to process the number of cells required for accurate population statistics (≧10,000 cells). High-throughput analysis of single cells is needed. However, existing techniques may be error-prone, expensive, slow, and labor-intensive, or may require expensive devices and control equipment.

The process is performed using a device comprising an array of chambers like those shown in FIGS. 18A through 18G. This device provides an inexpensive and robust platform that will rapidly isolate single cells, provide visual confirmation of successful trapping and lysis, and consume minimal reagents. The device provides several features that are advantageous to adapt PCR to high-throughput single-cell analysis. First, the device can individually isolate cells at a high success rate. This improves the sensitivity of the genetic analysis for rare cells. Second, the volume of solution in which cells are maintained is relatively small. The small solution volume prevents both dilution of the analyte and the introduction of contaminant DNA. Finally, the process can be carried out in a manner that does not require complex device components such as valves or mixers.

The steps of the cell capture and analysis process are performed as described herein with respect to FIGS. 18A through 18G. A digital waveform generator and a KEPCO Model BOP 1000M amplifier (to 1000V) is used to achieve the voltage and current requirements to scale up to tens of thousands of chambers. Several microfluidic chips can be run in rapid succession in order to analyze tens of thousands of single cells. Captured cells are further isolated by oil encapsulation. An optical system is used for rapid acquisition of multi-color fluorescent signal. The optical system comprises a mercury lamp source directing light to a rotating turret of excitation filters (tens of ms per color) under automated control. Fluorescent signal from the PCR array passes through emission filters to a sensitive CCD camera. Temperatures required for cell lysis and PCR cycling are controlled by an infrared light source. This optical system allows automated rapid read-out of the PCR array.

Experiments are performed using myeloid blast cell line K562, which carries the Bcr-Abl oncogene (to establish conditions) and cryopreserved AML. These cases can be readily screened for the most frequent mutations found in AML (e.g., FLT3, NPM1, NRAS). White blood cells (WBCs) are isolated from the sample through microcentrifugation and then washed several times to eliminate stray DNA. The WBCs are then resuspended in PCR reagent solution. The cell sample and PCR reagents are loaded into the trapping channels (FIG. 18C) under voltage control as described herein. In the case that additional PCR volume is needed, the design depicted in FIG. 18G can be employed. After pulling cells into chambers and isolating them with immiscible phase (FIGS. 18D and 18E), lysis and PCR cycling are ready to begin.

The PCR step is adapted for multi-color gene expression analysis. At a minimum, the PCR reagent mixture contains a hot start PCR Taq polymerase (one that is inhibited at low temperatures), primers specific for genes of interest, and appropriate fluorescent probes of various colors. The commercially available TaqMan probes (Applied Biosystems) are ideal for this application. The probes report replication of the target gene based on Förster resonance energy transfer (FRET). If a cell contains the mutant gene, fluorescent signal accumulates during each of 30-40 PCR cycles. Fluorescence is monitored with a highly sensitive optical instrument for reading out multi-color PCR. A mercury lamp serves as the excitation source and a filter turret that contains the three sets of dichroic and excitation filters selects the excitation wavelengths. The rotating turret can be controlled electronically allowing wavelengths to be selected in rapid sequence.

Hot start PCR is initialized by incubating for several minutes at ˜95° C. to remove inhibitor from polymerase and to lyse cells. Temperature of the entire chamber array is controlled by irradiation with infrared (IR) light. This method can be much faster than traditional heating methods (e.g., with resistive or peltier heaters). To ensure that the IR light does not interfere with fluorescence detection, a physical shutter blocks the IR source during the fluorescence collection and a long pass filter in front of the lamp blocks out visible radiation from illuminating the sample.

This process can be used to screen archived clinical AML samples in order to identify cells carrying a rare mutation and determine its frequency. The automated optical system enables rapid processing of arrays and the multi-color capability enables multi-gene analyses. This technology can be extended to examination of relapsed samples, revealing the selection that occurs under the pressure of chemotherapy. Moreover, this technology can in identifying genetic lesions associated with therapeutic resistance and therapy can be altered as resistant clones are detected.

### Example 5

### Characterization of nDEP and pDEP Cell Trapping and Application for Controlled Lysis

This example describes an exemplary process for characterizing nDEP and pDEP cell trapping. Trapping is performed using a single BPE in electrochemical contact with two parallel microfluidic channels (FIG. 7).

The device is fabricated using standard photolithographic techniques. Briefly, gold-coated glass slides are patterned with photoresist and wet etching of the gold. The BPE width is similar to a cell diameter (˜10-20 μm), and the exposed length of BPE in the microchannel is tens of microns as well. The microchannels are formed by pouring and curing polydimethylsiloxane (PDMS) on a photoresist patterned Si substrate. The microchannel dimensions are 20 μm tall×100 μm wide×1 cm long. The glass substrate and PDMS microchannels are aligned and reversibly bonded (e.g., by conformal contact) or irreversibly bonded (e.g., following oxygen plasma exposure). The devices are then filled and incubated with a dilute solution of ethylene glycol-propylene glycol block copolymer to coat the channel, dampen electroosmotic flow, and prevent adsorption of cells to the microchannel surface.

Solution conditions are chosen based on solution conductivity. While pDEP of cells uses low conductivity solutions, correct osmolarity is easily maintained with neutral species. Typical low conductivity dielectrophoresis solutions are comprised solely of 8.5% sucrose, 0.3% dextrose, and 0.75% bovine serum albumin (BSA). Additionally, cells with intact membranes are not damaged by the AC fields used for dielectrophoresis. However, the DC component of the electric field is more likely to cause damage, and as such, it is kept well below the electroporation threshold (˜100 kV/m).

TABLE 3
Experimental conditions for nDEP and pDEP cell trapping

| | BPE pole | AC field frequency | AC field amplitude | DC offset | [Tris buffer] |
|---|---|---|---|---|---|
| nDEP | anode | 1 kHz-100 kHz | 100 V-1000 V | 5 V-30 V | 100 mM |
| | | | peak-to-peak | | |
| pDEP | cathode | 1 MHz-15 MHz | 100 V-1000 V | 5 V-30 V | 10 mM + |
| | | | peak-to-peak | | sucrose |

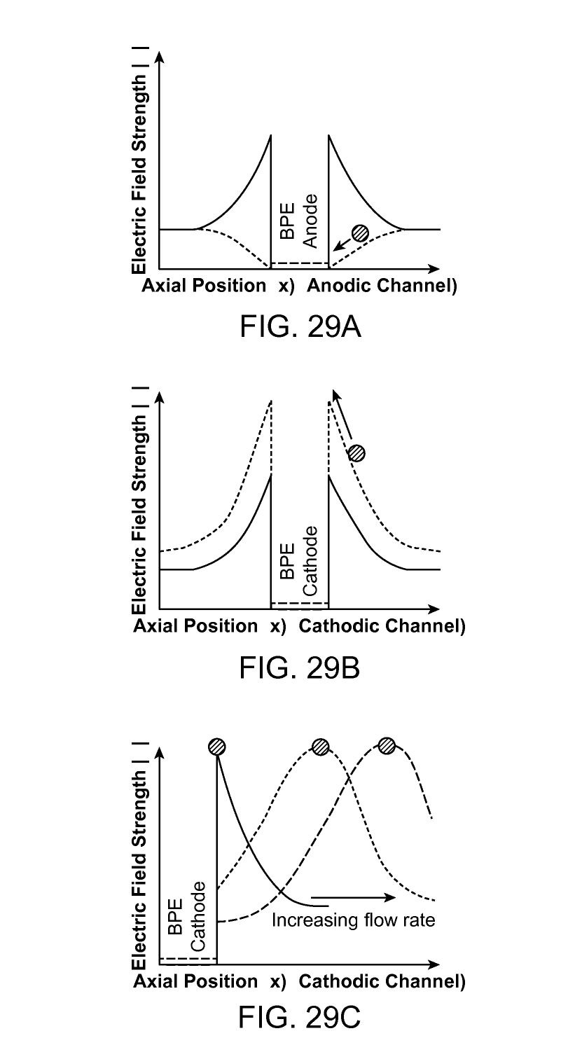

In this device, nDEP cell trapping proceeds at the BPE anode as follows. First, the microchannels are rinsed with 100 mM Tris buffer (pH 8.0). Second, the anodic microchannel is filled with a solution of B-cells (˜1×10⁵ cells/mL) in the same buffer by pressure driven flow (e.g., by gravity or syringe pump). Significantly, the viability of these cells in this buffer solution has been tested and it has been confirmed that cells are viable for at least 8 hrs (longest time tested). Finally, a combined AC and DC field with properties appropriate for nDEP of B-cells (as indicated in Table 3) is applied at V₁, V₂, and V₃ (FIG. 7) versus ground (V₄). Significantly, only a small portion of the total DC offset is dropped across the BPE in this configuration. A schematic depiction of the averaged axial electric field profile that develops along the anodic channel in the solution above the BPE anode is shown in FIG. 29A (solid line). Subsequently, the progression of the oxidation reaction described by eq. 1 causes the accumulation of ionic species around the BPE. This ion enrichment zone decreases E locally, leading to a new electric field profile (FIG. 29A, dashed line). Significantly, although E is zero above the BPE at all times (solid and dashed lines, FIG. 29A) cells can only be attracted to this region by nDEP after the ion enrichment zone forms (dashed line).

Sharper electric field gradients and stronger DEP forces form under pDEP trapping conditions at the BPE cathode. pDEP of B-cells occurs at higher frequencies and in low conductivity medium. First, the device is rinsed with 10 mM Tris buffer (pH 8.0) with added sucrose to prevent osmotic stress on the B-cells. As before, cell viability in this buffer solution has been confirmed. Second, the cathodic microchannel is filled with B-cells in the same solution. Finally, the combined AC and DC field described for pDEP in Table 3 is applied. The initial averaged axial electric field profile that develops over the BPE cathode is depicted in FIG. 29B (solid line). Over time, the production of OH— and its following reaction with the buffer cation, TrisH+(eqs. 2 and 3) leads to the depletion of ions surrounding the BPE. As a result, the axial electric field in the solution above the BPE rapidly become amplified (FIG. 29B, dashed line). Cells are strongly attracted along this steep electric field gradient to the narrow region with highest E. Significantly, the depletion zone extends this amplified electric field region and associated field gradient in the z-direction (channel height). The key advantage of this pDEP scheme is that decreased solution conductivity amplified F_{DEP} in the trapping region by simultaneously impacting E and K (via εₘ*).

The pDEP trapping scheme described herein can create a mobile trapping zone. A cell is trapped by pDEP at the peak of the ion depletion zone and pressure-driven flow is used to achieve controlled axial translation of the depletion zone and trapped cell (FIG. 29C). The key advantages of this technique are: 1) the cell trapping can be contactless because the peak of the depletion zone can be moved off-electrode. This feature can increase tolerance for high operating currents, leading to stronger traps and prevent non-specific adsorption of the cell to the electrode; and 2) the trapped cell can be moved to a “loading zone” for downstream analysis (e.g., a droplet generator).

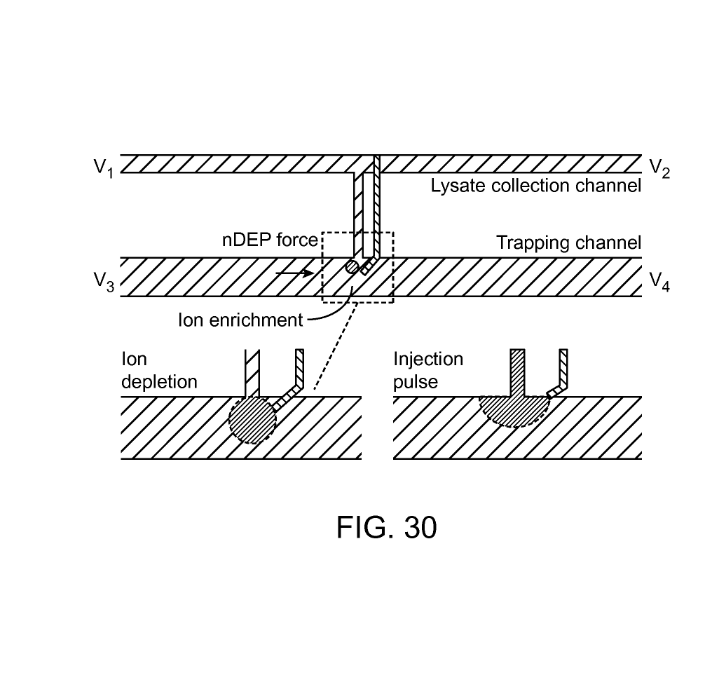

The nDEP cell trapping scheme described herein can be followed by membrane poration or lysis. In some cases, it may be desirable to achieve pores for transfection or to cause lysis for analysis of cytosolic components. A cell is sequentially trapped and then moved to conditions appropriate for poration or lysis. First, nDEP trapping conditions are employed to trap a cell. Significantly, no sucrose is added to the solution, and only the Tris buffer serves to balance osmotic pressure on the cell. Once the cell is trapped at the electrode (dashed line, FIG. 29A), the sign of the DC offset is switched to form a depletion zone over the same end of the BPE, where the cell is trapped (FIG. 29B, dashed line). The formation of the depletion zone, in the absence of sucrose puts osmotic pressure on the cell, causing it to swell and develop membrane pores. The applied DC voltage (degree of depletion) will control the degree and rate of swelling. A sufficiently high voltage can lead to lysis if desired. In this case, the contents of the trapped cell can be transported electrokinetically into a chamber or into a separate channel for droplet encapsulation and downstream processing (FIG. 30). Significantly, this technique combines label-free, cell-type-specific trapping with controlled membrane poration or lysis.

Dielectrophoresis can trap multiple cells by pearl chaining, a process in which cells are attracted to one another by dipole-dipole interaction. The number of cells captured is referred to in DEP as the yield (Y). Unlike many single cell technologies which rely on cell concentration (and Poisson statistics) to yield one cell, in dielectrophoresis, Y can be controlled through several experimental variables including the field frequency (ω), the trapping time, the viscosity of the medium, the fluid flow velocity, and the electric field strength (E). In the case that single cell capture cannot be completely controlled via experimental variables affecting Y, physical boundaries can be employed to limit the number of cells captured. For example, the trapping zone can be confined to a chamber similar in size to a single cell.

While preferred aspects of the present invention have been shown and described herein, it will be obvious to those skilled in the art that such aspects are provided by way of example only. Numerous variations, changes, and substitutions will now occur to those skilled in the art without departing from the invention. It should be understood that various alternatives to the aspects of the invention described herein can be employed in practicing the invention. It is intended that the following claims define the scope of the invention and that methods and structures within the scope of these claims and their equivalents be covered thereby.

What is claimed is:

1. A dielectrophoretic system comprising:
a fluidic containment structure comprising an ionically conductive phase;
a bipolar electrode having a portion situated within the fluidic containment structure, the portion being in electrical communication with the ionically conductive phase; and
a power source in electrical communication with the ionically conductive phase and configured to apply an electric field thereto, the electric field comprising an AC component having a frequency range from about 1 kHz to about 100 MHz and a voltage range from about 1 V to about 1 kV and a DC component having a voltage range from about 10 mV to about 100 V.

2. The dielectrophoretic system of claim 1, wherein the portion comprises a tip of the bipolar electrode.

3. The dielectrophoretic system of claim 1 or 2, wherein the power source is not in direct contact with the bipolar electrode.

4. The dielectrophoretic system of any one of claims 1-3, wherein the electric field comprises electric field minima or electric field maxima near the portion of the bipolar electrode.

5. The dielectrophoretic system of any one of claims 1-4, wherein the fluidic containment structure is a well.

6. The dielectrophoretic system of any one of claims 1-4, wherein the fluidic containment structure is a fluidic channel.

7. The dielectrophoretic system of claim 6, further comprising a second fluidic channel comprising a second ionically conductive phase, wherein a second portion of the bipolar electrode is situated in the second fluidic channel, the second ionically conductive phase is in electrical communication with the second portion of the bipolar electrode, and the power source is in electrical communication with the second ionically conductive phase.

8. The dielectrophoretic system of claim 7, wherein the portion comprises a first end of the bipolar electrode and the second portion comprises an opposing end of the bipolar electrode.

9. The dielectrophoretic system of claim 7 or 8, wherein the fluidic channel is fluidically isolated from the second fluidic channel.

10. The dielectrophoretic system of claim 7 or 8, wherein the fluidic channel and second fluidic channel are fluidly connected by a third fluidic channel, the third fluidic channel having a width smaller than a width of the fluidic channel and a width of the second fluidic channel

11. The dielectrophoretic system of any one of claims 6-8, wherein the fluidic channel comprises a channel wall and a chamber formed in the channel wall, and wherein the portion of the bipolar electrode is situated within the chamber.

12. The dielectrophoretic system of claim 11, wherein the chamber comprises a hydrophilic material.

13. The dielectrophoretic system of claim 11 or 12, wherein the bipolar electrode comprises a hydrophilic material.

14. The dielectrophoretic system of any one of claims 11-13, wherein the channel wall comprises a hydrophobic material.

15. The dielectrophoretic system of any one of claims 1-4, further comprising a plurality of bipolar electrodes.

16. The dielectrophoretic system of claim 15, wherein the fluidic containment structure is a fluidic channel and each of the plurality of bipolar electrodes has a portion situated in the fluidic channel and in electrical communication with the ionically conductive phase.

17. The dielectrophoretic system of claim 15, further comprising an array of wells each comprising an ionically conductive phase, wherein each of the plurality of bipolar electrodes has a portion situated in a different respective well of the plurality of wells.

18. The dielectrophoretic system of claim 17, wherein the portion of each of the plurality of bipolar electrodes is situated at a bottom surface of the different respective well.

19. The dielectrophoretic system of claim 15, further comprising a plurality of fluidic channels each fluidically isolated from each other and each comprising an ionically conductive phase, wherein each of the plurality of bipolar electrodes has a first portion situated in one of the plurality of fluidic channels and a second portion situated in another of the plurality of fluidic channels.

20. The dielectrophoretic system of any one of claims 1-19, further comprising a removal device configured to displace a sample situated near the bipolar electrode or the plurality of bipolar electrodes.

21. The dielectrophoretic system of any one of claims 1-20, further comprising a collection device configured to collect a sample situated near the bipolar electrode or the plurality of bipolar electrodes.

22. The dielectrophoretic system of any one of claims 1-21, further comprising a droplet generation device configured to generate a droplet comprising a sample situated near the bipolar electrode or the plurality of bipolar electrodes.

23. The dielectrophoretic system of any one of claims 1-22, further comprising a detection device configured to detect a sample situated near the bipolar electrode or the plurality of bipolar electrodes.

24. The dielectrophoretic system of any one of claims 20-23, wherein the sample comprises a biological cell trapped near the bipolar electrode or the plurality of bipolar electrodes.

25. The dielectrophoretic system of any one of claims 1-24, wherein the ionically conductive phase comprises an amplification reagent.

26. The dielectrophoretic system of claim 25, wherein the amplification reagent is selected from a polymerase chain reaction (PCR) reagent, rolling circle amplification (RCA) reagent, nucleic acid sequence based amplification (NASBA) reagent, loop-mediated amplification (LAMP) reagent, or a combination thereof.

27. The dielectrophoretic system of any one of claims 1-26, wherein the AC component has an electric field strength range selected from the following: from about 10 kV/m to about 1000 kV/m, from about 100 kV/m to about 1000 kV/m, from about 1000 kV/m to about 10 MV/m, or from about 1 MV/m to about 100 MV/m.

28. A method for manipulating an object comprising using the dielectrophoretic system of any one of claims 1-27 to manipulate the position of an object.

29. A fluidic device comprising:
a first fluidic channel comprising a first ionically conductive phase;
a second fluidic channel comprising a second ionically conductive phase;
a bipolar electrode comprising a first portion and a second portion, wherein the first portion is in electrical communication with the first ionically conductive phase and the second portion is in electrical communication with the second ionically conductive phase; and
a power source in electrical communication with the first and second ionically conductive phases and configured to apply an electric field comprising an AC component and a DC component to the first and second ionically conductive phases, the electric field comprising an electric field minimum or an electric field maximum near the first and second portions of the bipolar electrode.

30. The fluidic device of claim 29, wherein the AC component has a frequency range from about 1 kHz to about 100 MHz and a voltage range from about 1 V to about 1 kV and the DC component has a voltage range from about 10 mV to about 100 V.

31. The fluidic device of claim 29 or 30, wherein the power source is not in direct contact with the bipolar electrode.

32. The fluidic device of any one of claims 29-31, wherein the first portion comprises a first end of the bipolar electrode and the second portion comprises an opposing end of the bipolar electrode.

33. The fluidic device of any one of claims 29-32, wherein the first fluidic channel comprises a channel wall and a chamber formed in the channel wall, and wherein the first portion of the bipolar electrode is situated within the chamber.

34. The fluidic device of claim 33, wherein the chamber comprises a hydrophilic material.

35. The fluidic device of claim 33 or 34, wherein the bipolar electrode comprises a hydrophilic material.

36. The fluidic device of any one of claims 33-35, wherein the channel wall comprises a hydrophobic material.

37. The fluidic device of any one of claims 29-36, wherein the first and second fluidic channels are fluidically isolated from each other by an insulating barrier.

38. The fluidic device of any one of claims 29-36, wherein the first and second fluidic channels are fluidly connected by a third fluidic channel, the third fluidic channel having a width smaller than a width of the first fluidic channel and a width of the second fluidic channel.

39. The fluidic device of any one of claims 29-38, wherein the electric field minimum or the electric field maximum is generated by faradaic processes induced in the first and second portions of the bipolar electrode by the voltage.

40. The fluidic device of claim 34, wherein the faradaic processes produce a change in conductivity in a segment of the first ionically conductive phase near the first portion of the bipolar electrode.

41. The fluidic device of any one of claims 29-40, further comprising a plurality of bipolar electrodes each comprising a first portion in electrical communication with the first ionically conductive phase and a second portion in electrical communication with the second ionically conductive phase.

42. The fluidic device of any one of claims 29-41, wherein the AC component has an electric field strength range selected from the following: from about 10 kV/m to about 1000 kV/m, from about 100 kV/m to about 1000 kV/m, from about 1000 kV/m to about 10 MV/m, or from about 1 MV/m to about 100 MV/m.

43. A fluidic device comprising:
a plurality of fluidic containment structures each comprising an ionically conductive phase;
a plurality of bipolar electrodes each comprising a first portion and a second portion, wherein the first portion of each of the plurality of bipolar electrodes is in electrical communication with an ionically conductive phase of one of the plurality of fluidic containment structures and the second portion of each of the plurality of electrodes is in electrical communication with an ionically conductive phase of another of the plurality of fluidic containment structures; and
a power source configured to apply an electric field comprising an AC component and a DC component to each ionically conductive phase of the plurality of fluidic containment structures, the electric field comprising electric field minima or electric field maxima near the first and second portions of each of the plurality of bipolar electrodes.

44. The fluidic device of claim 43, wherein the AC component has a frequency range from about 1 kHz to about 100 MHz and a voltage range from about 1 V to about 1 kV and the DC component has a voltage range from about 10 mV to about 100 V.

45. The fluidic device of claim 43 or 44, wherein the power source is not in direct contact with any of the plurality of bipolar electrodes.

46. The fluidic device of any one of claims 43-45, wherein the first portion comprises a first end of the bipolar electrode and the second portion comprises an opposing end of the bipolar electrode.

47. The fluidic device of any one of claims 43-46, wherein at least some of the plurality of fluidic containment structures are fluidically isolated from each other by an insulating barrier.

48. The fluidic device of any one of claims 43-47, wherein the plurality of fluidic containment structures comprise an array of wells.

49. The fluidic device of claim 48, wherein the first portion is situated at a bottom surface of one of the array of wells and the second portion is situated at a bottom surface of another of the array of wells.

50. The fluidic device of any one of claims 43-47, wherein the plurality of fluidic containment structures comprise a plurality of fluidic channels.

51. The fluidic device of claim 50, wherein one of the plurality of fluidic channels comprises a channel wall and a chamber formed in the channel wall, and wherein the first portion of one of the plurality of bipolar electrodes is situated within the chamber.

52. The fluidic device of claim 51, wherein the chamber comprises a hydrophilic material.

53. The fluidic device of claim 51-52, wherein the one of the plurality of bipolar electrodes comprises a hydrophilic material.

54. The fluidic device of any one of claims 51-53, wherein the channel wall comprises a hydrophobic material.

55. The fluidic device of claim 50, wherein two of the plurality of fluidic channels are fluidly connected by a third fluidic channel, the third fluidic channel having a width smaller than a width of each of the two fluidic channels.

56. The fluidic device of any one of claims 43-55, wherein each of the electric field minima or the electric field maxima is generated by faradaic processes induced in the first and second portions of a corresponding one of the plurality of bipolar electrodes by the voltage.

57. The fluidic device of claim 56, wherein the faradaic processes produce a change in conductivity in a segment of the ionically conductive phase near the first portion of each of the plurality of bipolar electrodes.

58. The fluidic device of any one of claims 43-57, wherein the AC component has an electric field strength range selected from the following: from about 10 kV/m to about 1000 kV/m, from about 100 kV/m to about 1000 kV/m, from about 1000 kV/m to about 10 MV/m, or from about 1 MV/m to about 100 MV/m.

59. A method for manipulating an object comprising using the fluidic device of any one of claims 29-58 to manipulate the position of an object.

60. A method for manipulating an object, the method comprising:
providing a fluidic containment structure comprising an ionically conductive phase and a bipolar electrode comprising a portion in electrical communication with the ionically conductive phase;
applying an electric field comprising an AC component and DC component to the ionically conductive phase, wherein the electric field comprises an electric field minimum or an electric field maximum near the portion of the bipolar electrode;
introducing an object into the ionically conductive phase; and
manipulating the position of the object within the ionically conductive phase using the electric field minimum or electric field maximum.

61. The method of claim 51, wherein the AC component has a frequency range from about 1 kHz to about 100 MHz and a voltage range 1 V to about 1 kV and the DC voltage component has a voltage range from about 10 mV to about 100 V.

62. The method of claim 60 or 61, wherein the object is uncharged.

63. The method of any one of claims 60-62, wherein the object is polarizable.

64. The method of any one of claims 60-63, wherein the object is a particle.

65. The method of any one of claims 60-64, wherein the object is a discrete phase.

66. The method of any one of claims 60-65, wherein the object is a biological cell or part of a biological cell.

67. The method of any one of claims 60-66, wherein manipulating the object comprises attracting the object towards the portion of the bipolar electrode.

68. The method of any one of claims 60-66, wherein manipulating the object comprises repelling the object away from the portion of the bipolar electrode.

69. The method of any one of claims 60-66, wherein manipulating the object comprises trapping the object within a segment of the ionically conductive phase.

70. The method of claim 69, wherein the segment comprises an ion depletion zone in the ionically conductive phase.

71. The method of claim 70, further comprising encapsulating the segment and the object within a droplet.

72. The method of claim 70, further comprising flowing the ionically conductive phase so as to manipulate the position of the segment and the object trapped in the segment.

73. The method of any one of claims 60-72, wherein the portion comprises a tip of the bipolar electrode.

74. The method of any one of claims 60-73, wherein the fluidic containment structure is a well.

75. The method of any one of claims 60-73, wherein the fluidic containment structure is a fluidic channel.

76. The method of claim 75, further comprising providing a second fluidic channel comprising a second ionically conductive phase, wherein the bipolar electrode comprises a second portion in electrical communication with the second ionically conductive phase.

77. The method of claim 76, wherein the portion comprises a first end of the bipolar electrode and the second portion comprises an opposing end of the bipolar electrode.

78. The method of claim 76 or 77, wherein the fluidic channel is fluidically isolated from the second fluidic channel.

79. The method of claim 76 or 77, wherein the fluidic channel and the second fluidic channel are fluidly connected by a third fluidic channel, the third fluidic channel having a width smaller than a width of the fluidic channel and a width of the second fluidic channel.

80. The method of any one of claims 75-79, wherein the portion of the bipolar electrode is situated near a branch point fluidly connecting the fluidic channel to a plurality of outlet channels.

81. The method of claim 80, further comprising applying a voltage across one of the plurality of outlet channels, thereby attracting the object into said one of the plurality of outlet channels.

82. The method of any one of claims 75-81, wherein the fluidic channel comprises a channel wall and a chamber formed in the channel wall, and wherein the portion of the bipolar electrode is situated within the chamber.

83. The method of claim 82, wherein the chamber comprises a hydrophilic material.

84. The method of claim 82 or 83, wherein the bipolar electrode comprises a hydrophilic material.

85. The method of any one of claims 82-84, wherein the channel wall comprises a hydrophobic material.

86. The method of any one of claims 82-85, wherein manipulating the position of the object comprises attracting the object into the chamber.

87. The method of claim 86, further comprising flowing a fluid that is immiscible with the ionically conductive phase into the fluidic channel, thereby forming a droplet within the chamber, the droplet comprising a segment of the ionically conductive phase and the object.

88. The method of claim 87, further comprising displacing the droplet from the chamber.

89. The method of any one of claims 60-88, further comprising introducing a plurality of objects into the ionically conductive phase and manipulating the plurality of objects within the ionically conductive phase using the electric field minimum or electric field maximum.

90. The method of any one of claims 60-86, further comprising introducing an amplification reagent into the fluidic containment structure.

91. The method of claim 90, wherein the amplification reagent is selected from a polymerase chain reaction (PCR) reagent, rolling circle amplification (RCA) reagent, nucleic acid sequence based amplification (NASBA) reagent, loop-mediated amplification (LAMP) reagent, or a combination thereof.

92. The method of any one of claims 60-91, further comprising detecting the presence or absence of an analyte.

93. The method of claim 92, wherein the detection comprises imaging.

94. The method of claim 93, wherein the imaging is performed using confocal microscopy, spinning disk microscopy, multi-photon microscopy, planar illumination microscopy, Bessel beam microscopy, differential interference contrast microscopy, phase contrast microscopy, epifluorescent microscopy, bright field imaging, dark field imaging, oblique illumination, or a combination thereof.

95. The method of any one of claims 60-94, wherein the AC component has an electric field strength range selected from the following: from about 10 kV/m to about 1000 kV/m, from about 100 kV/m to about 1000 kV/m, from about 1000 kV/m to about 10 MV/m, or from about 1 MV/m to about 100 MV/m.
